(19) United States
(12) Patent Application Publication
(10) Pub. No.: US 2025/0236852 A1
(43) Pub. Date: Jul. 24, 2025

(54) LARGE SERINE RECOMBINASES, SYSTEMS AND USES THEREOF

(71) Applicant: Beam Therapeutics Inc., Cambridge, MA (US)

(72) Inventors: Zharko DANILOSKI, Cambridge, MA (US); David BORN, Cambridge, MA (US); Yi YU, Cambridge, MA (US)

(21) Appl. No.: 19/079,568

(22) Filed: Mar. 14, 2025

Related U.S. Application Data
(63) Continuation of application No. PCT/US2023/074298, filed on Sep. 15, 2023.
(60) Provisional application No. 63/407,487, filed on Sep. 16, 2022.

(51) Int. Cl. C12N 9/12; C12N 15/86

(52) U.S. Cl. CPC C12N 9/1241; C12N 15/86; C12N 2740/15043; C12N 2750/14143

(57) ABSTRACT

The present invention provides novel serine recombinases, recombinase based systems and compositions, and methods for genomic targeting and modification. In some aspects, the large serine recombinases, and systems thereof are used to treat human diseases.

### CROSS-REFERENCE TO RELATED APPLICATIONS

This application is a Continuation Application of International Application No. PCT/US2023/074298, filed on Sep. 15, 2023, which claims priority to U.S. Provisional Patent Application Ser. No. 63/407,487, filed on Sep. 16, 2022, the contents of each of which are incorporated by reference herein in entirety for all purposes.

### SEQUENCE LISTING

The instant application contains a Sequence Listing which has been submitted electronically in XML file format and is hereby incorporated by reference in its entirety. Said XML copy, created on Dec. 8, 2022, is named BEM-017USP1_SL.xml and is 4,968,123 bytes in size.

### BACKGROUND

Recombinases, e.g. large serine recombinases (LSRs) catalyze the insertion and integration of DNA elements into genomes using site-specific recombination between short DNA “attachment sites”. For example, LSRs carry out integration between attachment sites in the phage (attP) and in the host bacteria (attB). LSRs are highly site-specific and highly directional. Excision between the product attL and attR sites does not occur in the absence of a phage-encoded recombination directionality factor.

Large serine recombinases that recognize and target specific sequences, can be used to repair genetic mutations, integrate functional genes, or localize enzymes or transcription factors to specific sites on the genome, allowing genetic and epigenetic regulation and transcriptional modulation through a variety of mechanisms. Precise genomic modification is a challenge in a wide variety of target genes. The simplicity, site-selectivity and strong directionality of the LSRs provide precise genomic modifications, advancing genetic engineering applications and gene therapy in a wide variety of organisms.

### SUMMARY OF THE INVENTION

The present invention provides novel large serine recombinases, among other things, systems and compositions comprising one or more large serine recombinases, and methods of use thereof for LSR mediated genome modifications. The enzymes, systems, cells and compositions of the present invention can be used as therapeutic agents for treatment of diseases, as well as research tools to study precise genomic modifications in a host cell, tissue or subject, in vivo or in vitro.

In one aspect, the present invention provides a system for modifying DNA, the system comprising: (a) a large serine recombinase having at least 70% identity to any one of the amino acid sequences of SEQ ID NOs: 1-774; (b) a DNA recognition sequence comprising an attP or an attB site; and/or (c) a heterologous nucleic acid sequence.

In some embodiments, the large serine recombinase comprises an amino acid sequence having at least 80%, 85%, 90%, 95%, 96%, 97%, 98% or 99% identity to any one of the amino acid sequences of SEQ ID NOs: 1-774.

In some embodiments, the large serine recombinase comprises an amino acid sequence having at least 90% identity to any one of the amino acid sequences of SEQ ID NOs: 1-774. In some embodiments, the large serine recombinase comprises an amino acid sequence having at least 95% identity to any one of the amino acid sequences of SEQ ID NOs: 1-774. In some embodiments, the large serine recombinase comprises an amino acid sequence having at least 99% identity to any one of the amino acid sequences of SEQ ID NOs: 1-774.

In some embodiments, the large serine recombinase comprises an amino acid sequence selected from the amino acid sequences of SEQ ID NOs: 1-774.

In some embodiments, the large serine recombinase is encoded by a polynucleotide having at least 70% identity to any one of polynucleotide sequences of SEQ ID NOs: 775-1548.

In some embodiments, the large serine recombinase is encoded by a polynucleotide having at least 80%, 85%, 90%, 95%, 96%, 97%, 98% or 99% identity to any one of the polynucleotide sequences of SEQ ID NOs: 775-1548.

In some embodiments, the large serine recombinase is encoded by a polynucleotide having at least 90% identity to any one of the polynucleotide sequences of SEQ ID NOs: 775-1548. In some embodiments, the large serine recombinase is encoded by a polynucleotide having at least 95% identity to any one of the polynucleotide sequences of SEQ ID NOs: 775-1548. In some embodiments, the large serine recombinase is encoded by a polynucleotide having at least 99% identity to any one of the polynucleotide sequences of SEQ ID NOs: 775-1548.

In some embodiments, the large serine recombinase is encoded by a polynucleotide selected from any one of the polynucleotide sequences of SEQ ID NOs: 775-1548.

In some embodiments, the large serine recombinase is derived from a phage, bacterial genome, a virus, an archaea, a fungi, a eukaryotic genome (e g., human microbiome). In some embodiments, the large serine recombinase is derived from a phage genome. In some embodiments, the large serine recombinase is derived from a bacterial genome. In some embodiments, an engineered, non-naturally occurring serine recombinase modified from a phage, bacterial genome, a virus, a fungi, a eukaryotic genome (e g., human microbiome), is provided herein. In some embodiments, the serine recombinase is codon-optimized.

In some embodiments, the system comprises an attP site that recognizes a cognate attB site in the genome and causes recombination integrating the heterologous DNA in the genome.

In some embodiments, the system comprises an attB site that recognizes a cognate attP site in the genome and causes recombination integrating the heterologous DNA in the genome.

In some embodiments, the interaction of the attP site and the attB site mediates integration of the heterologous DNA sequence into the genome.

In some embodiments, the attP or attB site comprises a parapalindromic sequence.

In some embodiments, the attP or attB sites are naturally occurring, i.e., pseudo attP or pseudo attB sites.

In some embodiments, the attP or attB sites are engineered or optimized for expression in a target cell.

In some embodiments, the heterologous DNA sequence is recombined or inserted into the target genome at one or more attP or attB sites.

In some embodiments, the heterologous DNA sequence is recombined or inserted into the target genome at a single attP or attB site.

In some embodiments, the system is comprised in one or more integrative vectors.

In some embodiments, the system is comprised in a single integrative vector.

In one embodiment, a vector comprising the system described herein is provided.

In one embodiment, the vector is a plasmid vector or a viral vector.

In some embodiments, the vector is an adenoviral vector, an adeno associated viral (AAV) vector, a lentiviral vector, a retroviral vector or a rabies virus vector. In some embodiments, the vector is an adenoviral vector. In some embodiments, the vector is an AAV vector. In some embodiments, the vector is a lentiviral vector. In some embodiments, the vector is a retroviral vector. In some embodiments, the vector is a rabies virus vector. In some embodiments, more than one vector is used for packaging the system. In some embodiments, more than one AAV vector is used for packaging the system.

In some embodiments, the vector is non-viral vector. In some embodiments, non-viral delivery is using a lipid nanoparticle (LNP).

In some embodiments, the system comprises mRNA encoding a large serine recombinase. In some embodiments, the system further comprises a heterologous donor sequence. In some embodiments, the heterologous donor sequence is DNA. In some embodiments, the DNA is double-stranded. In some embodiments, the donor sequence is a circular double-stranded DNA. In some embodiments, the donor sequence is a linear double-stranded DNA. In some embodiments, the linear dsDNA is converted to circular double-stranded DNA in cells. In some embodiments, the heterologous donor sequence is single-stranded DNA. In some embodiments, the heterologous donor sequence is mRNA. In some embodiments, the single-stranded donor sequence is converted to circular double-stranded DNA in cells. In some embodiments, the RNA donor sequence is converted to circular double-stranded DNA in cells.

In some aspects, provided herein is a method for modifying a genome in a cell, the method comprising: contacting the cell with a polynucleotide encoding a serine recombinase enzyme having at least 70% identity to any one of the amino acid sequences of SEQ ID NOs: 1-774, a DNA recognition sequence comprising a first and a second attachment site; and a heterologous DNA sequence; wherein the serine recombinase enzyme mediates site-specific recombination between the first and the second attachment site causing integration of heterologous DNA, thereby modifying the genome.

In some embodiments, at least one DNA recognition site is a pseudo attachment site. In some embodiments, one or more DNA recognition sites is an engineered site. In some embodiments, the first and second attachment sites are attP or attB sites. In some embodiments, the attB site is in a target genome and the attP site sequence is in an integrative vector. In some embodiments, the attP site sequence is in a target genome and the attB site sequence is in an integrative vector.

In some embodiments, the site-specific recombination occurs at one or more sites in the cell.

In some embodiments, the site-specific recombination occurs at a single site in the cell.

In some embodiments, the site-specific recombination results in expression of a heterologous gene.

In some embodiments, the recombination is carried out in a mammalian cell. In some embodiments, the recombination is carried out in a human cell.

In some embodiments, the recombination is carried out in a cell line. In some embodiments, the recombination is carried out in a primary cell.

In some embodiments, the recombination is carried out in a non-dividing cell.

In some embodiments, the recombination is carried out in a dividing cell.

In some embodiments, the recombination is carried out in immune cells, such as T cells, B cells, macrophages, NK cells, etc., stem cells, progenitor cells, or cancer cells.

In some embodiments, the recombination is carried out in vivo. In some embodiments, the in vivo recombination treats a genetic disease by repairing a genetic deficiency and/or restoring a functional gene. In some embodiments, the in vivo recombination treats a cancer by delivering a lethal or conditional lethal gene. In some embodiments, the in vivo recombination results in genome editing by introducing one or more enzymes selected from a group consisting of a Cas enzyme, a base editor, deaminase and a reverse transcriptase.

In some embodiments, the serine recombinase directs stable integration of the heterologous DNA. In some embodiments, the serine recombinase directs reversible integration of the heterologous DNA. In some embodiments, the heterologous DNA further comprises a Recombinase Directionality Factor (RDF) leading to excision of integrated DNA from the genome.

In some embodiments, the expression of large serine recombinase in the present system is regulated by a promoter. In some embodiments; the promoter is constitutive or inducible. In some embodiments; the promoter is constitutive. In some embodiments, the promoter is inducible. In some embodiments, the promoter sequence is a eukaryotic or viral promoter.

In some embodiments, the heterologous DNA integrated is between about 100 bp to about 20 kb in length, 1 kb to 10 kb in length, or 2 kb to 10 kb in length, or 2 kb to 40 kb in length.

In some embodiments, the present invention provides an engineered cell produced by the methods described herein.

In some embodiments, provided herein is a method of treating a genetic disease or cancer, wherein the engineered cell is administered to a patient in need thereof.

In some embodiments, the attP attachment site comprises between 30 to 75 contiguous nucleotides from any one of SEQ ID NOs: 1549-2322, corresponding to its cognate LSR sequence as described in Table 3.

### BRIEF DESCRIPTION OF THE DRAWING

Drawings are for illustration purposes only; not for limitation.

FIG. 1A is a graph that shows recombination or integration activity of exemplary large serine recombinases by relative GFP expression.

FIG. 1B is a graph that shows identification of exemplary pseudo attB sites in the human genome.

FIG. 2 is a graph that shows percent integration as GFP positive cells, in cells treated with varying amounts of plasmid donor (e.g., 50 ng or 200 ng) and varying amounts of LSR mRNA (e.g., 0, 10, 25, 50, 100 or 200 ng).

FIG. 3 is a graph that shows percent integration as GFP positive cells, in cells treated with varying amounts of LSR mRNA (0, 100, 250, 500, 1000 or 2000 ng) and DNA donor (e.g., 1 μg, 2 μg or 3 μg).

FIG. 4 is a graph that shows percent integration as GFP positive cells, in cells treated with varying amounts of LSR mRNA (2 μg) and donor DNA (e.g. 0.25 μg, 0.5 μg, 1 μg, 2 μg).

### DETAILED DESCRIPTION

### Definitions

In order for the present invention to be more readily understood, certain terms are first defined below. Additional definitions for the following terms and other terms are set forth throughout the specification.

A or An: The articles “a” and “an” are used herein to refer to one or to more than one (i.e., to at least one) of the grammatical object of the article. By way of example, “an element” means one element or more than one element.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: Two events or entities are “associated” with one another, as that term is used herein, if the presence, level and/or form of one is correlated with that of the other. For example, a particular entity (e.g., polypeptide) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Biologically active: As used herein, the phrase “biologically active” refers to a characteristic of any agent that has activity in a biological system, and particularly in an organism. For instance, an agent that, when administered to an organism, has a biological effect on that organism, is considered to be biologically active. In particular embodiments, where a peptide is biologically active, a portion of that peptide that shares at least one biological activity of the peptide is typically referred to as a “biologically active” portion.

Base editor: By “base editor (BE),” or “nucleobase editor (NBE)” is meant an agent that binds a polynucleotide and has nucleobase modifying activity. In various embodiments, the base editor comprises a nucleobase modifying polypeptide (e.g., a deaminase) and a polynucleotide programmable nucleotide binding domain in conjunction with a guide polynucleotide (e.g., guide RNA). The base editor has base editing activity, i.e., a domain capable of modifying a base (e.g., A, T, C, G, or U) within a nucleic acid molecule (e.g., DNA). In some embodiments, the base editor is capable of deaminating one or more bases within a DNA molecule. In some embodiments, the base editor is capable of deaminating a cytosine (C) or an adenosine (A) within DNA. In some embodiments, the base editor is capable of deaminating a cytosine (C) and an adenosine (A) within DNA. In some embodiments, the base editor is a cytidine base editor (CBE). In some embodiments, the base editor is an adenosine base editor (ABE). In some embodiments, the base editor is an adenosine base editor (ABE) and a cytidine base editor (CBE). In some embodiments, the base editor is a nuclease-inactive Cas9 (dCas9) fused to an adenosine deaminase. In some embodiments, the base editor is fused to an inhibitor of base excision repair, for example, a UGI domain, or a dISN domain. In some embodiments, the fusion protein comprises a Cas9 nickase fused to a deaminase and an inhibitor of base excision repair, such as a UGI or dISN domain. In other embodiments the base editor is an abasic base editor. Details of base editors are described in International PCT Application Nos. PCT/2017/045381 (WO2018/027078) and PCT/US2016/058344 (WO2017/070632), each of which is incorporated herein by reference for its entirety.

Base editing activity: As used herein the term “base editing activity” is meant acting to chemically alter a base within a polynucleotide. In one embodiment, a first base is converted to a second base. In one embodiment, the base editing activity is cytidine deaminase activity, e.g., converting target C⋅G to T⋅A. In another embodiment, the base editing activity is adenosine or adenine deaminase activity, e.g., converting A⋅T to G⋅C. In another embodiment, the base editing activity is cytosine or cytidine deaminase activity, e.g., converting target C⋅G to T⋅A and adenosine or adenine deaminase activity, e.g., converting A⋅T to G⋅C.

Cleavage: As used herein, cleavage refers to a break in a target nucleic acid created by a nuclease of a CRISPR system described herein. In some embodiments, the cleavage event is a double-stranded DNA break. In some embodiments, the cleavage event is a single-stranded DNA break. In some embodiments, the cleavage event is a single-stranded RNA break. In some embodiments, the cleavage event is a double-stranded RNA break.

Complementary: As used herein, complementary refers to a nucleic acid strand that forms Watson-Crick base pairing, such that A base pairs with T, and C base pairs with G, or non-traditional base pairing with bases on a second nucleic acid strand. In other words, it refers to nucleic acids that hybridize with each other under appropriate conditions.

Enzyme: The term “enzyme” as defined herein encompasses native as well as modified enzymes. The term “native” as used herein refers to a material recovered from a source in nature as distinct from material artificially modified or altered by man in the laboratory. For example, a native enzyme is encoded by a gene that is present in the genome of a wild-type organism or cell. By contrast, a modified or engineered enzyme is encoded by a nucleic acid molecule that has been modified in the laboratory so as to differ from the native polypeptide, e.g. by insertion, deletion or substitution of one or more amino acid(s) or any combination of these possibilities. A genome modifying enzyme refers to any enzyme that can modify a genome in a host organism and/or a host cell.

Ex Vivo: As used herein, the term “ex vivo” refers to events that occur in cells or tissues, grown outside rather than within a multi-cellular organism.

Functional equivalent or analog: As used herein, the term “functional equivalent” or “functional analog” denotes, in the context of a functional derivative of an amino acid sequence, a molecule that retains a biological activity (either function or structural) that is substantially similar to that of the original sequence. A functional derivative or equivalent may be a natural derivative or is prepared synthetically. Exemplary functional derivatives include amino acid sequences having substitutions, deletions, or additions of one or more amino acids, provided that the biological activity of the protein is conserved. The substituting amino acid desirably has chemico-physical properties which are similar to that of the substituted amino acid. Desirable similar chemico-physical properties include, similarities in charge, bulkiness, hydrophobicity, hydrophilicity, and the like.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

Inhibition: As used herein, the terms “inhibition,” “inhibit” and “inhibiting” refer to processes or methods of decreasing or reducing activity and/or expression of a protein or a gene of interest. Typically, inhibiting a protein or a gene refers to reducing expression or a relevant activity of the protein or gene by at least 10% or more, for example, 20%, 30%, 40%, or 50%, 60%, 70%, 80%, 90% or more, or a decrease in expression or the relevant activity of greater than 1-fold, 2-fold, 3-fold, 4-fold, 5-fold, 10-fold, 50-fold, 100-fold or more as measured by one or more methods described herein or recognized in the art.

Genome modification: As used herein, the term “modification” or “modifying’ or “modified” when applied to nucleic acid sequences, refers to any change to the sequences within the genome, such as single nucleotide variant (SNV), insertion, deletion, site specific recombination, substitution, chromosomal translocation and structural variation (SV), etc. For example, in terms of insertion, the sequence modification may be the integration of a transgene into a target genomic site. For example, for a target genomic sequence, the donor DNA comprises a sequence complementary, identical, or homologous to the target genomic sequence and a sequence modification region.

Hybridization: As used herein, the term “hybridization” refers to a reaction in which two or more nucleic acids bind with each other via hydrogen bonding by Watson-Crick pairing, Hoogstein binding or other sequence-specific binding between the bases of the two nucleic acids. A sequence capable of hybridizing with another sequence is termed the “complement” of the sequence, and is said to be “complementary” or show “complementarity”.

Indel: As used herein, the term “indel” refers to insertion or deletion of bases in a nucleic acid sequence. It commonly results in mutations and is a common form of genetic variation.

In Vitro: As used herein, the term “in vitro” refers to events that occur in an artificial environment, e.g., in a test tube or reaction vessel, in cell culture, etc., rather than within a multi-cellular organism.

In Vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Large serine recombinase: As used herein, the large serine recombinases (LSRs) are a family of enzymes, often encoded in temperate phage genomes or on mobile elements. Large serine recombinases can catalyze the movement of DNA elements into and out of a host genome (e.g., bacterial chromosomes) using site-specific recombination between short DNA “attachment sites” such as the attachment sites in the phage genome (attP site) and the attachment sites in the bacterial genome (attB site), allowing precisely to cut and recombine DNA in a highly controllable and predictable way.

Linker: The term “linker” refers to any means, entity or moiety used to join two or more entities. In some embodiments, the linker is a covalent linker. In some embodiments, the linker is a non-covalent linker. Examples of covalent linkers include covalent bonds or a linker moiety covalently attached to one or more of the proteins or domains to be linked, In some embodiments, the linker is a non-covalent bond, e.g., an organometallic bond through a metal center such as platinum atom. The joining can be permanent or reversible. For covalent linkages, various functionalities can be used, such as amide groups, including carbonic acid derivatives, ethers, esters, including organic and inorganic esters, amino, urethane, urea and the like. To provide for linking, the domains can be modified by oxidation, hydroxylation, substitution, reduction etc. to provide a site for coupling. Methods for conjugation are well known by persons skilled in the art and are encompassed for use in the present invention. Linker moieties include, but are not limited to, chemical linker moieties, or for example a peptide linker moiety (a linker sequence). It will be appreciated that modification which do not significantly decrease the function of the RNA-binding domain and effector domain are preferred.

Mutation: As used herein, the term “mutation” has the ordinary meaning in the art, and includes, for example, point mutations, substitutions, insertions, deletions, inversions, and deletions.

Oligonucleotide: As used herein, the term “oligonucleotide” generally refers to polynucleotides of between about 5 and about 100 nucleotides of single-or double-stranded DNA. Oligonucleotides are also known as “oligomers” or “oligos” and may be isolated from genes, or chemically synthesized.

Polypeptide: The term “polypeptide” as used herein refers to a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. As is known to those skilled in the art, polypeptides may be processed and/or modified. As used herein, the terms “polypeptide” and “peptide” are used inter-changeably.

Prevent: As used herein, the term “prevent” or “prevention”, when used in connection with the occurrence of a disease, disorder, and/or condition, refers to reducing the risk of developing the disease, disorder and/or condition.

Protein: The term “protein” as used herein refers to one or more polypeptides that function as a discrete unit. If a single polypeptide is the discrete functioning unit and does not require permanent or temporary physical association with other polypeptides in order to form the discrete functioning unit, the terms “polypeptide” and “protein” may be used interchangeably. If the discrete functional unit is comprised of more than one polypeptide that physically associate with one another, the term “protein” refers to the multiple polypeptides that are physically coupled and function together as the discrete unit.

Recombination: As used herein the term “recombination” or “recombination reaction” refers to a change of a nucleic acid molecule including, for example, one or more nucleic acid strand breaks (e.g., a double-strand break), followed by joining of two nucleic acid strand ends (e.g., sticky ends). In some instances, the recombination reaction comprises insertion of an insert nucleic acid, e.g., into a target site, e.g., in a genome or a construct. In some instances, the recombination reaction comprises flipping or reversing of a nucleic acid, e.g., in a genome or a construct. In some instances, the recombination reaction comprises removing a nucleic acid, e.g., from a genome or a construct.

Recognition sequence: A recognition sequence (e.g., DNA recognition sequence) generally refers to a nucleic acid (e.g., DNA) sequence that is recognized (e.g., capable of being bound by) a genome modifying enzyme, e.g., a serine recombinase. In the context of serine recombinase, a recognition sequence comprises two recognition sequences, one that is positioned in the integration site (the site into which a nucleic acid is to be integrated) and another adjacent a nucleic acid of interest to be introduced into the integration site. The recognition sequences are generically referred to as attP and attB. Recognition sequences can be native or altered relative to a native sequence. The recognition sequence may vary in length, but typically ranges from about 20 nt to about 200 nt, from about 30 to 90 nt, more usually from 30 to 70 nt. In some embodiments, the attP attachment site comprises between 30 to 75 contiguous nucleotides.

Subject: The term “subject”, as used herein, means any subject for whom diagnosis, prognosis, or therapy is desired. For example, a subject can be a mammal, e.g., a human or non-human primate (such as an ape, monkey, orangutan, or chimpanzee), a dog, cat, guinea pig, rabbit, rat, mouse, horse, cattle, or cow.

Substantial identity: The phrase “substantial identity” is used herein to refer to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215(3): 403-410, 1990; Altschul, et al., Methods in Enzymology; Altschul et al., Nucleic Acids Res. 25:3389-3402, 1997; Baxevanis et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

The terms “specific” or “specificity” as used herein refers to the property of having a degree of preference for recognizing, binding, hybridizing, recombining, or reacting with a desired target or substrate versus one or more non-desired targets or substrates under the conditions tested or specified. In general, the terms “specific for” or having “specificity for” is used to refer to a preference of at least 50% for the desired target or substrate versus two or more non-desired targets or substrates collectively.

Target Nucleic Acid: The term “target nucleic acid” as used herein refers to nucleotides of any length (oligonucleotides or polynucleotides) to which the large serine recombinase system binds. Target nucleic acids may have three-dimensional structure, may including coding or non-coding regions, may include exons, introns, mRNA, tRNA, rRNA, siRNA, shRNA, miRNA, ribozymes, cDNA, plasmids, vectors, exogenous sequences, endogenous sequences. A target nucleic acid can comprise modified nucleotides, include methylated nucleotides, or nucleotide analogs. A target nucleic acid may be interspersed with non-nucleic acid components. A target nucleic acid is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

Therapeutically effective amount: As used herein, the term “therapeutically effective amount” refers to an amount of a therapeutic molecule (e.g., an engineered LSR described herein) which confers a therapeutic effect on a treated subject, at a reasonable benefit/risk ratio applicable to any medical treatment. The therapeutic effect may be objective (i.e., measurable by some test or marker) or subjective (i.e., subject gives an indication of or feels an effect). In particular, the “therapeutically effective amount” refers to an amount of a therapeutic molecule or composition effective to treat, ameliorate, or prevent a particular disease or condition, or to exhibit a detectable therapeutic or preventative effect, such as by ameliorating symptoms associated with the disease, preventing or delaying the onset of the disease, and/or also lessening the severity or frequency of symptoms of the disease. A therapeutically effective amount can be administered in a dosing regimen that may comprise multiple unit doses. For any particular therapeutic molecule, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular subject may depend upon a variety of factors including the disorder being treated and the severity of the disorder; the activity of the specific pharmaceutical agent employed; the specific composition employed; the age, body weight, general health, sex and diet of the subject; the time of administration, route of administration, and/or rate of excretion or metabolism of the specific therapeutic molecule employed; the duration of the treatment; and like factors as is well known in the medical arts.

Treatment: As used herein, the term “treatment” (also “treat” or “treating”) refers to any administration of a therapeutic molecule (e.g., a Site specific recombinase protein or system described herein) that partially or completely alleviates, ameliorates, relieves, inhibits, delays onset of, reduces severity of and/or reduces incidence of one or more symptoms or features of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition.

### Site-Specific Recombinases

Site specific recombinases catalyze breaking and rejoining of DNA strands at specific locations in a genome, thereby bringing about precise genetic rearrangements. Using recombinase-medicated genetic rearrangements benefits the understanding of genetic mechanisms of diseases and advances gene therapy as well. There are two large families of site specific recombinases: serine recombinases and tyrosine recombinases. Serine recombinases precisely manipulate genomic sequences and DNA molecules.

Serine recombinases (such as large serine recombinases) can be found in many bacteriophages and bacterial genomes. The identification of novel large serine recombinases with specificity for unique attachment sites (attP and attB) allows for the expansion of the available tools for genome modulation, allowing for precise targeting of diverse sites. The present invention is based, in part, on the surprising discovery that novel serine recombinase enzymes isolated from different phage genomes, coupled with specific attachment sequences (e.g., attP), which recognize cognate attachment sites in the host genome (e.g., attB) can be engineered for expression in eukaryotic cells (e.g., human, plant, etc.). Accordingly, the described serine recombinase enzymes and their variants are functional in eukaryotes. Described herein is use of engineered serine recombinase enzymes in human cells with diverse attP or attB recognition sequences to target various genomic sites and integrate or recombine heterologous genes. Additionally, the present invention provides methods of use of newly identified LSRs for genome modifications in connection with gene therapy.

In some embodiments, the attP site comprises between 30 to 75 contiguous nucleotides from any one of SEQ ID NOs: 1549-2322, corresponding to its cognate LSR sequence as described in Table 3.

Accordingly, a system comprising a large serine recombinase (LSR) is provided in the present invention; the LSR system can be used for modifying a DNA sequence in a genome. In some aspects, the system comprises: (a) a large serine recombinase having at least 70% identity to any one of the amino acid sequences of SEQ ID NOs: 1-774; (b) a DNA recognition sequence comprising an attP and/or an attB site; and/or (c) a heterologous DNA sequence. Methods of use of the present LSRs and LSR containing systems to modify a host genome (e.g., a host cell) are also provided. In some aspects, the method comprises introducing into the host cell a LSR or a system comprising a LSR as described herein and a heterologous nucleic acid sequence.

### Large Serine Recombinases

In some aspects, the enzyme of the system for modifying a nucleic acid sequence in a genome is a serine recombinase, e.g., a large serine recombinase (LSR). The terms “large serine recombinases” also refers to “serine integrases” interchangeably. The large serine recombinase can be derived from any suitable organism, such as viruses, bacteria including bacteriophages that infect bacteria, archaea, fungi, mammals including human (e.g., human microbiomes). Described herein are large serine recombinase proteins obtained from phages or bacterial genomes. In some embodiments, the large serine recombinase is identified from a bacteriophage.

Accordingly, the present invention provides serine recombinase polypeptides (e.g., any one of SEQ ID NOs: 1-774) that can be used to modify or manipulate a DNA sequence, e.g., by recombining two DNA sequences comprising cognate recognition sequences (e.g., attP or attB sequences) that can be bound by the recombinase polypeptide. In some embodiments, the large serine recombinase described herein comprises an amino acid sequence having at least 70% (e.g., 70%, 75%, 80%, 81%, 82%, 83%, 84%, 85%, 86%, 87%, 88%, 89%, 90%, 91%, 92%, 93%, 94%, 95%, 96%, 97%, 98%, 99% or more) identity to any one of SEQ ID NOs: 1-774. In some embodiments, a large serine recombinase described herein comprises an amino acid sequence having at least 70% identity to any one of SEQ ID NOs: 1-774. In some embodiments, a large serine recombinase described herein comprises an amino acid sequence having at least 75% identity to any one of SEQ ID NOs: 1-774. In some embodiments, a large serine recombinase described herein comprises an amino acid sequence having at least 80% identity to any one of SEQ ID NOs: 1-774. In some embodiments, a large serine recombinase described herein comprises an amino acid sequence having at least 85% identity to any one of SEQ ID NOs: 1-774. In some embodiments, a large serine recombinase described herein comprises an amino acid sequence having at least 90% identity to any one of SEQ ID NOs: 1-774. In some embodiments, a large serine recombinase described herein comprises an amino acid sequence having at least 95% identity to any one of SEQ ID NOs: 1-774. In some embodiments, a large serine recombinase described herein comprises an amino acid sequence having at least 96% identity to any one of SEQ ID NOs: 1-774. In some embodiments, a large serine recombinase described herein comprises an amino acid sequence having at least 97% identity to any one of SEQ ID NOs: 1-774. In some embodiments, a large serine recombinase described herein comprises an amino acid sequence having at least 98% identity to any one of SEQ ID NOs: 1-774. In some embodiments, a large serine recombinase described herein comprises an amino acid sequence having at least 99% identity to any one of SEQ ID NOs: 1-774. In some embodiments, the amino acid sequence of a large serine recombinase protein is identical to any one of SEQ ID NOs: 1-774.

In some embodiments, a variant of a large serine recombinase as described herein is provided. In some embodiments, the variant comprises an amino acid substitution or chemical modifications of one or more amino acids. In other embodiments, the variant comprises the catalytic domain of a large serine recombinase as described herein. In some exemplary embodiments, a variant of a large serine recombinase comprises a truncation at the N-terminus, C-terminus, or both the N- and C-termini relative to the amino acid sequence of any one of SEQ ID NOs: 1-774. In some embodiments, the truncated variant has 1, 2, 3, 4, 5, 6, 7, 8, 9, 10, 15, 20, 25, 30, 35, 40, 45, or 50 amino acids deleted from the N-terminus or the C-terminus.

In some embodiments, a recombinase described herein is fused to a heterologous domain, e.g., a heterologous DNA binding domain to form a recombinant enzyme. In some embodiments, a recombinase is fused to a heterologous DNA binding domain, e.g., a DNA binding domain from a zinc finger, TAL, meganuclease, transcription factor, or sequence-guided DNA binding element. In some embodiments, a recombinase is fused to a DNA binding domain from a sequence-guided DNA binding element, e.g., a CRISPR-associated (Cas) DNA binding element, e.g., a Cas9.

In some embodiments, the sequences of any one of SEQ ID NOs: 1-1548 further comprise a nuclear localization sequence (NLS). In some embodiments, the NLS sequence is a prefix sequence preceding SEQ ID NOs: 1-774 and SEQ ID NOs.: 775-1548. In some embodiments, the NLS comprises a sequence having 70%, 75%, 80%, 85%, 90%, 95%, 99% or greater identity to GCCACCATGCCCAAGAAGAAGCGGAAGGTT (SEQ ID NO: 2323). In some embodiments, the NLS consists of a sequence having 100% identity to SEQ ID NO: 2323.

In some embodiments, any one of sequences in SEQ ID NOs: 1-1548 further comprise a sequence comprising an NLS, SV40 transcriptional terminator, sequences flanking the LSR sequence, comprising upstream and downstream sequences comprising attP or attB sites separated by a spacer. In some embodiments, the sequences further comprise a barcode sequence. In some embodiments, the attP (or attB) site within the flanking sequence is about 30-75 bp in length. In some embodiments, the attP (or attB) site comprises at least about 30-75 bp from SEQ ID NOs: 1549-2322.

In some embodiments, the present invention provides a polynucleotide sequence that encodes any one of the large serine recombinases described herein. A representative nucleic acid sequence for each large serine recombinase (LSR) can be found in any one of SEQ ID NOs.: 775-1548.

In some embodiments, the large serine recombinase described herein is encoded by a polynucleotide having a nucleic acid sequence at least 70% (e.g., 70%, 75%, 80%, 81%, 82%, 83%, 84%, 85%, 86%, 87%, 88%, 89%, 90%, 91%, 92%, 93%, 94%, 95%, 96%, 97%, 98%, 99% or more) identical to any one of SEQ ID NO: 775-1548. In some embodiments, a large serine recombinase described herein is encoded by a polynucleotide having a nucleic acid sequence at least 70% identical to any one of SEQ ID NOs.: 775-1548. In some embodiments, a large serine recombinase described herein is encoded by a polynucleotide having a nucleic acid sequence at least 75% identical to any one of SEQ ID NOs.: 775-1548. In some embodiments, a large serine recombinase described herein is encoded by a polynucleotide having a nucleic acid sequence at least 80% identical to any one of SEQ ID NOs.: 775-1548. In some embodiments, a large serine recombinase described herein is encoded by a polynucleotide having a nucleic acid sequence at least 85% identical to any one of SEQ ID NOs.: 775-1548. In some embodiments, a large serine recombinase described herein is encoded by a polynucleotide having a nucleic acid sequence at least 90% identical to any one of SEQ ID NOs.: 775-1548. In some embodiments, a large serine recombinase described herein is encoded by a polynucleotide having a nucleic acid sequence at least 95% identical to any one of SEQ ID NOs.: 775-1548. In some embodiments, a large serine recombinase described herein is encoded by a polynucleotide having a nucleic acid sequence of any one of SEQ ID NOs.: 775-1548.

In some embodiments, the polynucleotide encoding a large serine recombinase of the present invention is codon optimized. Various species exhibit codon bias (i.e. differences in codon usage by organisms) which correlates with the efficiency of translation of messenger RNA (mRNA) by utilizing codons in mRNA that correspond with the abundance of tRNA species for that codon in a particular organism. Various methods in the art can be used for computer optimization, including for example through use of software. In some embodiments, codon optimization refers to modification of nucleic acid sequences for enhanced expression in the host cells of interest by replacing at least one codon (e.g. 1, 2, 3, 4, 5, 10, 15, 20, 25, 50 or more codons) of the native sequence with codons that are more frequently used or most frequently used in the genes of the host cell while maintaining the native amino acid sequence. This type of optimization is known in the art and entails the mutation of foreign-derived DNA to mimic the codon preferences of the intended host organism or cell while encoding the same protein. Thus, the codons are changed, but the encoded protein remains unchanged. Codon optimization improves soluble protein levels and increases activity and editing efficiency in a given species. Codon optimization also results in increased translation and protein expression.

In some embodiments, the large serine recombinase protein is codon optimized for expression in eukaryotic cells. In some embodiments, the large serine recombinase protein is codon optimized for expression in human cells. In some embodiments, the large serine recombinase protein is codon optimized for expression in human immune cells. In some embodiments, the large serine recombinase protein is codon optimized for expression in human T-cells.

In some embodiments, the LSR encoding polynucleotide comprises at least one nucleotide modification, including any chemical modifications, e.g., modification of nucleosides and sugar subunits.

In some embodiments, the large serine recombinase is a recombinant polypeptide variant. In some embodiments, a LSR variant comprises a modified catalytic domain, or a modified nucleic acid binding domain, or a combination of the above. In some embodiments, a LSR variant comprises a catalytic domain of any one of the large serine recombinases of any one of SEQ ID NOs: 1-774. In some embodiments, the LSR recombinant polypeptide comprises at least one substitution of amino acid residues of any one of SEQ ID Nos: 1-774.

In some embodiments, a LSR variant comprises a catalytic domain encoded by the polynucleotide sequence of any one of the large serine recombinases in SEQ ID NOs: 775-1548.

In some embodiments, the LSR variant is a recombinant polypeptide that comprises a domain that contains recombinase activity derived from any one of SEQ ID Nos: 1-774, and a DNA binding domain that binds to or is capable of binding to a recognition sequence. In other embodiments, the LSR variant is a recombinant polypeptide that comprises a domain that contains recombinase activity and a DNA binding domain derived from any one of SEQ ID Nos: 1-774, that binds to or is capable of binding to a recognition sequence.

In some embodiments, the LSR variant is a recombinant polypeptide that comprises a domain that contains recombinase activity derived from any one of codon-optimized polynucleotide sequences provided in SEQ ID Nos: 775-1548, and a DNA binding domain that binds to or is capable of binding to a recognition sequence. In other embodiments, the LSR variant is a recombinant polypeptide that comprises a domain that contains recombinase activity and a DNA binding domain derived from any one of codon-optimized polynucleotide sequences provided in SEQ ID Nos: 775-1548, that binds to or is capable of binding to a recognition sequence.

In some embodiments, a large serine recombinase is fused to nuclear localization sequences, including, but not limited to, an NLS of the SV40 large T antigen, nucleoplasmin, c-myc, hRNPA1 M9, IBB domain from importin-alpha, NLS of myoma T protein, human p53, c-abl IV, influenza virus NS1, hepatitis virus delta antigen, mouse Mx1, human poly(ADP-ribose) polymerase, steroid hormone receptor (human) glucocorticoid. In some embodiments, the NLS is fused to the N-terminus of a LSR or variant thereof. In some embodiments, the NLS is fused to the C-terminus of a LSR or variant thereof. In some embodiments, a large serine recombinase protein is fused to epitope tags including, but not limited to, hemagglutinin (HA) tags, histidine (His) tags, FLAG tags, Myc tags, V5 tags, VSV-G tags, SNAP tags, thioredoxin (Trx) tags.

In some embodiments, a large serine recombinase is fused to reporter genes including, but not limited to, glutathione-S-transferase (GST), horseradish peroxidase (HRP), chloramphenicol transferase (CAT), HcRed, DsRed, cyan fluorescent protein, yellow fluorescent protein and blue fluorescent protein, green fluorescent protein (GFP), including enhanced versions or superfolded GFP, as well as other modified versions of reporter genes.

In some embodiments, serum half-life of an engineered large serine recombinase protein is increased by fusion with heterologous proteins including, but not limited to, a human serum albumin protein, transferrin protein, human IgG and/or sialylated peptide, such as the carboxy-terminal peptide (CTP, of chorionic gonadotropin β chain).

In some embodiments, serum half-life of an engineered large serine recombinase protein is decreased by fusion with destabilizing domains, including, but not limited to, geminin, ubiquitin, FKBP12-L106P, and/or dihydrofolate reductase.

### Determination of LSR Activity

In accordance with the present invention, a novel LSR polypeptide can be validated using any methods known in the art. In some embodiments, a LSR is tested using a two-vector system in which the LSR enzyme is expressed in an expressing vector and the specific recognition site sequences that is recognizable by the LSR and donor nucleic acid molecule are included in a separated vector. In other embodiments, a novel

LSR polypeptide can be validated using a single one vector system in which the LSR and its recognition site sequences are integrated in a single vector; the detailed description of the one-vector for identifying an active large serine recombinase is described in detail in the applicant's copending patent application.

### Attachment Sites (AttP or AttB)

Large serine recombinases or integrases carry out recombination between attachment sites on the phage and bacterial genomes (i.e., target genomes), known as attP and attB, respectively. Each large serine recombinase binds to its target sequence only in the presence of a specific sequence, known as an attachment site in the target genome such as a bacterial genome (attB). Large serine recombinases isolated from different phage or bacterial species recognize (i.e., bind to) different attP or attB sequences. Thus, locations in the genome that can be targeted by different large serine recombinase proteins are limited by the locations of unique attP or attB sequences, leading to specificity of genome modification.

Accordingly, in some aspects, the LSR system as described herein comprises a recognition site sequence to which the LSR in the system specifically binds. The recognition site sequence, in some embodiments, comprises an attP site sequence. In some embodiment, the recognition sequence comprises an attB site sequence. In other embodiments, the recognition sequence comprises an attP sequence and an attB sequence.

In some embodiments, the recognition site sequence comprises about 10-200 nucleotides (nt), about 20-200 nt, about 20-150 nt, about 20-100 nt, about 20-80 nt, 25-150 nt, 25-100 nt, 25-80 nt, 30-150 nt, 30-100 nt, or 30-75 nt. In some embodiments, the recognition site sequence comprises about 30-75 nt. In some examples, the recognition site sequence comprises about 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, 35 nt, 36 nt, 37 nt, 38 nt, 39nt, 40 nt, 41 nt, 42 nt, 43 nt, 44 nt, 45 nt, 46 nt, 47 nt, 48 nt, 49 nt, 50 nt, 51 nt, 52 nt, 53 nt, 54 nt, 55 nt, 56 nt, 57 nt, 58 nt, 59 nt, 60 nt, 61 nt, 62 nt, 63 nt, 64 nt, 65 nt, 66 nt, 67 nt, 68 nt, 69 nt, 70 nt, 71 nt, 72 nt, 73 nt, 74 nt, 75 nt, 80 nt, 85 nt, 90 nt, 95 nt or 100 nt.

In some embodiments, the specific attP sequence is a sequence located within about 500 base pairs flanking the coding sequence of the large serine recombinase in the phage genome. In some embodiments, the specific attP sequence is a sequence located within about 450 base pairs flanking the coding sequence of the large serine recombinase in the phage genome. In some embodiments, the specific attP sequence is a sequence located within about 400 base pairs flanking the coding sequence of the large serine recombinase in the phage genome. In some embodiments, the specific attP sequence is a sequence located within about 350 base pairs flanking the coding sequence of the large serine recombinase in the phage genome. In some embodiments, the specific attP sequence is a sequence located within about 300 base pairs flanking the coding sequence of the large serine recombinase in the phage genome. In some embodiments, the specific attP sequence is a sequence located within about 250 base pairs flanking the coding sequence of the large serine recombinase in the phage genome. In some embodiments, the specific attP sequence is a sequence located within about 200 base pairs flanking the coding sequence of the large serine recombinase in the phage genome. In some embodiments, the specific attP sequence is a sequence located within about 150 base pairs flanking the coding sequence of the large serine recombinase in the phage genome. In some embodiments, the specific attP sequence is a sequence located within about 100 base pairs flanking the coding sequence of the large serine recombinase in the phage genome. In some embodiments, the specific attP sequence is a sequence located within about 50 base pairs flanking the coding sequence of the large serine recombinase in the phage genome. In some embodiments, the sequence flanking the coding sequence of the large serine recombinase refers to the sequence upstream of the coding sequence of the large serine recombinase. In some embodiments, the sequence flanking the coding sequence of the large serine recombinase refers to the sequence downstream of the coding sequence of the large serine recombinase.

In some embodiments, the specific attB sequence is a sequence located within about 500 base pairs flanking the coding sequence of the large serine recombinase in the phage genome. In some embodiments, the specific attB sequence is a sequence located within about 450 base pairs flanking the coding sequence of the large serine recombinase in the phage genome. In some embodiments, the specific attB sequence is a sequence located within about 400 base pairs flanking the coding sequence of the large serine recombinase in the phage genome. In some embodiments, the specific attB sequence is a sequence located within about 350 base pairs flanking the coding sequence of the large serine recombinase in the phage genome. In some embodiments, the specific attB sequence is a sequence located within about 300 base pairs flanking the coding sequence of the large serine recombinase in the phage genome. In some embodiments, the specific attB sequence is a sequence located within about 250 base pairs flanking the coding sequence of the large serine recombinase in the phage genome. In some embodiments, the specific attB sequence is a sequence located within about 200 base pairs flanking the coding sequence of the large serine recombinase in the phage genome. In some embodiments, the specific attB sequence is a sequence located within about 150 base pairs flanking the coding sequence of the large serine recombinase in the phage genome. In some embodiments, the specific attB sequence is a sequence located within about 100 base pairs flanking the coding sequence of the large serine recombinase in the phage genome. In some embodiments, the specific attB sequence is a sequence located within about 50 base pairs flanking the coding sequence of the large serine recombinase in the phage genome. In some embodiments, the sequence flanking the coding sequence of the large serine recombinase refers to the sequence upstream of the coding sequence of the large serine recombinase. In some embodiments, the sequence flanking the coding sequence of the large serine recombinase refers to the sequence downstream of the coding sequence of the large serine recombinase.

In some embodiments, the attP sequence is a naturally occurring attP sequence. In some embodiments, the attP site is an engineered variant. In some embodiments, the attP comprises one or more substitutions. In some embodiments, the attB sequence is a naturally occurring attP sequence. In some embodiments, the attB site is an engineered variant. In some embodiments, the attB comprises one or more substitutions. In some examples, the attP site sequence in the system comprises a sequence having at least 30%, 35%, 40%, 45%, 50%, 55%, 56%, 60%, 65%, 70%, 75%, 80%, 85%, 90%, 95%, 99% or greater identity to a naturally occurring attP sequence. In some examples, the attB sequence in the system comprises a sequence having at least 30%, 35%, 40%, 45%, 50%, 55%, 56%, 60%, 65%, 70%, 75%, 80%, 85%, 90%, 95%, 99% or greater identity to a naturally occurring attB sequence.

In some embodiments, the attP sequence and/or the attB sequence of the present system comprises an engineered recognition sequence.

In some embodiments, the attP sequence comprises two portions of recognition sequences, a first portion of the recognition sequence and a second portion recognition sequence. In some embodiments, the attB sequence comprises two portions of recognition sequences, a first portion of the recognition sequence and a second portion of the recognition sequence. The first and second portions of the attP sequence interact with the first and second portions of the attB sequence. The LSR binds to the attP-attB complex to mediate site specific recombination.

The first portion of the attP recognition sequence, in some embodiments, comprises a parapalindromic nucleic acid sequence. The first portion of the attB recognition sequence, in some embodiments, comprises a parapalindromic nucleic acid sequence. As used herein, the term ‘parapalindromic” means that one sequence is a palindrome relative to the other sequence or has at least 20%, 30%, 40%, 50%, 60%, 70%, 75%, 80%, 85%, 90%, 95%, 96%, 97%, 98%, or 99% sequence identity to a palindrome relative to the other sequence. In some embodiments, the second portion of the attP recognition sequence comprises parapalindromic nucleic acid sequence. Each of the parapalindromic sequence comprises about 10-40 nt, 10-35nt, 10-30nt, 15-40nt, 15-35 nt, or 20-30 nt. The first portion of the attB recognition sequence, in some embodiments, comprises a parapalindromic nucleic acid sequence. In some embodiments, the second portion of the attB recognition sequence comprises parapalindromic nucleic acid sequence. Each of the parapalindromic sequence comprises about 10-40 nt, 10-35 nt, 10-30 nt, 15-40 nt, 15-35 nt, or 20-30 nt.

In some embodiments, the attP sequence of the present system further comprises a core sequence, wherein the core sequence is located between the first portion and the second portion of the attP recognition sequence. In other embodiments, the attB sequence of the present system further comprises a core sequence, wherein the core sequence is located between the first portion and the second portion of the attB recognition sequence. In some instances, a core sequence can be cleaved by a recombinase.

The core sequence within the attP sequence or within the attB sequence comprises about 2-20 nt, e.g., 2 nt, 3 nt, 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, or 20 nt. In some embodiments, the core sequence of the attB and attP are identical. In some embodiments, the core sequence of the attB and attP are not identical, e.g., have less than 99, 95, 90, 80, 70, 60, 50, 40, 30, or 20% identity. As a non-limiting example, an attP sequence is typically arranged from the 5′ end to the 3′end as follows: a first portion of the recognition sequence, a core sequence and a second portion of the recognition sequence. As another non-limiting example, an attB sequence is typically arranged from the 5′ end to the 3′end as follows: a first portion of the recognition sequence, a core sequence and a second portion of the recognition sequence.

In some embodiments, the attP sequence of the large serine recombinase system recombines with a cognate attB sequence in the target genome, integrating heterologous nucleic acid molecule. In some embodiments, the attB sequence is a naturally occurring attB site sequence in the target genome. In some embodiments, the attB sequence is a pseudo attB sequence.

In some embodiments, an attB sequence may be introduced into a host genome using a gene editing system, e.g., a base editor. In some embodiments, an attP sequence may be introduced into a host genome using a gene editing system, e.g., a base editor.

In some embodiments, the attB sequence of the large serine recombinase system recombines with a cognate attP sequence in the target genome, integrating heterologous DNA. In some embodiments, the attP sequence is a naturally occurring attP site sequence in the target genome. In some embodiments, the attP sequence is a pseudo attP sequence.

In some embodiments, the attP sequence of a LSR system and the cognate attB sequence comprises the same nucleic acid sequence. In other embodiments, the attP sequence of a LSR system and the cognate attB sequence do not comprises the same nucleic acid sequences. As non-limiting examples, the attP sequence has about 70%, 75%, 80%, 85%, 90%, 95% 96%, 97%, 98%, or 99% identity to its cognate attB sequence.

Accordingly, the large serine recombinase described herein exhibits activity, for example, recombination or integration in the presence of a unique attB and attP sequence leading to genome modification.

In some embodiments, each large serine recombinase described herein does not bind or exhibit activity with other attP or attB sequences, except for the specific attP and attB sequence it recognizes. Any one of SEQ ID NOs: 1549-2322 shows flanking sequences comprising attP sites for cognate LSR sequences as described in Table 3.

TABLE 3
Sequences identifying LSR and cognate flanking sequence comprising attP or
attB, and sequence identifier from the Gut Phage Genome database (Camarillo-Guerrero
et al., Massive expansion of human gut bacteriophage diversity; Cell, 2021, 184: 1098-1109;
http://ftp.ebi.ac.uk/pub/databases/metagenomics/genome_sets/gut_phage_database).

| | | Sequences | |
|---|---|---|---|
| | | flanking LSR | |
| LSR Amino Acid | Codon Optimized | comprising attP | Sequence |
| Sequences | LSR ORF | sites | Identifier |
| SEQ ID NO: 1 | SEQ ID NO: 775 | SEQ ID NO: 1549 | NC_002656 |
| SEQ ID NO: 2 | SEQ ID NO: 776 | SEQ ID NO: 1550 | ASN69149.1 |
| SEQ ID NO: 3 | SEQ ID NO: 777 | SEQ ID NO: 1551 | WP_109962774.1 |
| SEQ ID NO: 4 | SEQ ID NO: 778 | SEQ ID NO: 1552 | QIW89333.1 |
| SEQ ID NO: 5 | SEQ ID NO: 779 | SEQ ID NO: 1553 | uvig_401611 |
| SEQ ID NO: 6 | SEQ ID NO: 780 | SEQ ID NO: 1554 | uvig_576757 |
| SEQ ID NO: 7 | SEQ ID NO: 781 | SEQ ID NO: 1555 | uvig_205537 |
| SEQ ID NO: 8 | SEQ ID NO: 782 | SEQ ID NO: 1556 | uvig_281475 |
| SEQ ID NO: 9 | SEQ ID NO: 783 | SEQ ID NO: 1557 | uvig_22285 |
| SEQ ID NO: 10 | SEQ ID NO: 784 | SEQ ID NO: 1558 | uvig_274113 |
| SEQ ID NO: 11 | SEQ ID NO: 785 | SEQ ID NO: 1559 | uvig_176095 |
| SEQ ID NO: 12 | SEQ ID NO: 786 | SEQ ID NO: 1560 | ivig_2328 |
| SEQ ID NO: 13 | SEQ ID NO: 787 | SEQ ID NO: 1561 | uvig_594158 |
| SEQ ID NO: 14 | SEQ ID NO: 788 | SEQ ID NO: 1562 | uvig_181433 |
| SEQ ID NO: 15 | SEQ ID NO: 789 | SEQ ID NO: 1563 | uvig_154782 |
| SEQ ID NO: 16 | SEQ ID NO: 790 | SEQ ID NO: 1564 | uvig_569447 |
| SEQ ID NO: 17 | SEQ ID NO: 791 | SEQ ID NO: 1565 | uvig_187460 |
| SEQ ID NO: 18 | SEQ ID NO: 792 | SEQ ID NO: 1566 | uvig_166991 |
| SEQ ID NO: 19 | SEQ ID NO: 793 | SEQ ID NO: 1567 | uvig_169676 |
| SEQ ID NO: 20 | SEQ ID NO: 794 | SEQ ID NO: 1568 | uvig_284816 |
| SEQ ID NO: 21 | SEQ ID NO: 795 | SEQ ID NO: 1569 | uvig_366143 |
| SEQ ID NO: 22 | SEQ ID NO: 796 | SEQ ID NO: 1570 | uvig_121245 |
| SEQ ID NO: 23 | SEQ ID NO: 797 | SEQ ID NO: 1571 | uvig_190766 |
| SEQ ID NO: 24 | SEQ ID NO: 798 | SEQ ID NO: 1572 | uvig_152630 |
| SEQ ID NO: 25 | SEQ ID NO: 799 | SEQ ID NO: 1573 | uvig_500555 |
| SEQ ID NO: 26 | SEQ ID NO: 800 | SEQ ID NO: 1574 | uvig_356689 |
| SEQ ID NO: 27 | SEQ ID NO: 801 | SEQ ID NO: 1575 | uvig_527188 |
| SEQ ID NO: 28 | SEQ ID NO: 802 | SEQ ID NO: 1576 | uvig_415064 |
| SEQ ID NO: 29 | SEQ ID NO: 803 | SEQ ID NO: 1577 | uvig_593675 |
| SEQ ID NO: 30 | SEQ ID NO: 804 | SEQ ID NO: 1578 | uvig_200526 |
| SEQ ID NO: 31 | SEQ ID NO: 805 | SEQ ID NO: 1579 | uvig_188594 |
| SEQ ID NO: 32 | SEQ ID NO: 806 | SEQ ID NO: 1580 | uvig_323580 |
| SEQ ID NO: 33 | SEQ ID NO: 807 | SEQ ID NO: 1581 | uvig_81430 |
| SEQ ID NO: 34 | SEQ ID NO: 808 | SEQ ID NO: 1582 | uvig_395648 |
| SEQ ID NO: 35 | SEQ ID NO: 809 | SEQ ID NO: 1583 | uvig_255494 |
| SEQ ID NO: 36 | SEQ ID NO: 810 | SEQ ID NO: 1584 | ivig_2835 |
| SEQ ID NO: 37 | SEQ ID NO: 811 | SEQ ID NO: 1585 | uvig_78894 |
| SEQ ID NO: 38 | SEQ ID NO: 812 | SEQ ID NO: 1586 | uvig_205989 |
| SEQ ID NO: 39 | SEQ ID NO: 813 | SEQ ID NO: 1587 | uvig_580229 |
| SEQ ID NO: 40 | SEQ ID NO: 814 | SEQ ID NO: 1588 | uvig_94393 |
| SEQ ID NO: 41 | SEQ ID NO: 815 | SEQ ID NO: 1589 | uvig_401826 |
| SEQ ID NO: 42 | SEQ ID NO: 816 | SEQ ID NO: 1590 | uvig_183461 |
| SEQ ID NO: 43 | SEQ ID NO: 817 | SEQ ID NO: 1591 | uvig_19322 |
| SEQ ID NO: 44 | SEQ ID NO: 818 | SEQ ID NO: 1592 | uvig_539751 |
| SEQ ID NO: 45 | SEQ ID NO: 819 | SEQ ID NO: 1593 | uvig_408451 |
| SEQ ID NO: 46 | SEQ ID NO: 820 | SEQ ID NO: 1594 | uvig_154620 |
| SEQ ID NO: 47 | SEQ ID NO: 821 | SEQ ID NO: 1595 | uvig_349562 |
| SEQ ID NO: 48 | SEQ ID NO: 822 | SEQ ID NO: 1596 | uvig_596853 |
| SEQ ID NO: 49 | SEQ ID NO: 823 | SEQ ID NO: 1597 | uvig_4360 |
| SEQ ID NO: 50 | SEQ ID NO: 824 | SEQ ID NO: 1598 | uvig_167506 |
| SEQ ID NO: 51 | SEQ ID NO: 825 | SEQ ID NO: 1599 | uvig_339756 |
| SEQ ID NO: 52 | SEQ ID NO: 826 | SEQ ID NO: 1600 | uvig_182703 |
| SEQ ID NO: 53 | SEQ ID NO: 827 | SEQ ID NO: 1601 | ivig_3237 |
| SEQ ID NO: 54 | SEQ ID NO: 828 | SEQ ID NO: 1602 | uvig_297200 |
| SEQ ID NO: 55 | SEQ ID NO: 829 | SEQ ID NO: 1603 | uvig_470108 |
| SEQ ID NO: 56 | SEQ ID NO: 830 | SEQ ID NO: 1604 | uvig_32054 |
| SEQ ID NO: 57 | SEQ ID NO: 831 | SEQ ID NO: 1605 | uvig_399343 |
| SEQ ID NO: 58 | SEQ ID NO: 832 | SEQ ID NO: 1606 | uvig_290255 |
| SEQ ID NO: 59 | SEQ ID NO: 833 | SEQ ID NO: 1607 | uvig_242919 |
| SEQ ID NO: 60 | SEQ ID NO: 834 | SEQ ID NO: 1608 | uvig_138748 |
| SEQ ID NO: 61 | SEQ ID NO: 835 | SEQ ID NO: 1609 | uvig_448583 |
| SEQ ID NO: 62 | SEQ ID NO: 836 | SEQ ID NO: 1610 | uvig_596866 |
| SEQ ID NO: 63 | SEQ ID NO: 837 | SEQ ID NO: 1611 | uvig_42013 |
| SEQ ID NO: 64 | SEQ ID NO: 838 | SEQ ID NO: 1612 | uvig_452057 |
| SEQ ID NO: 65 | SEQ ID NO: 839 | SEQ ID NO: 1613 | ivig_4185 |
| SEQ ID NO: 66 | SEQ ID NO: 840 | SEQ ID NO: 1614 | uvig_58086 |
| SEQ ID NO: 67 | SEQ ID NO: 841 | SEQ ID NO: 1615 | uvig_75655 |
| SEQ ID NO: 68 | SEQ ID NO: 842 | SEQ ID NO: 1616 | uvig_442715 |
| SEQ ID NO: 69 | SEQ ID NO: 843 | SEQ ID NO: 1617 | ivig_244 |
| SEQ ID NO: 70 | SEQ ID NO: 844 | SEQ ID NO: 1618 | uvig_271148 |
| SEQ ID NO: 71 | SEQ ID NO: 845 | SEQ ID NO: 1619 | uvig_460604 |
| SEQ ID NO: 72 | SEQ ID NO: 846 | SEQ ID NO: 1620 | uvig_171430 |
| SEQ ID NO: 73 | SEQ ID NO: 847 | SEQ ID NO: 1621 | uvig_585929 |
| SEQ ID NO: 74 | SEQ ID NO: 848 | SEQ ID NO: 1622 | uvig_120053 |
| SEQ ID NO: 75 | SEQ ID NO: 849 | SEQ ID NO: 1623 | uvig_365399 |
| SEQ ID NO: 76 | SEQ ID NO: 850 | SEQ ID NO: 1624 | uvig_432464 |
| SEQ ID NO: 77 | SEQ ID NO: 851 | SEQ ID NO: 1625 | uvig_204911 |
| SEQ ID NO: 78 | SEQ ID NO: 852 | SEQ ID NO: 1626 | uvig_97244 |
| SEQ ID NO: 79 | SEQ ID NO: 853 | SEQ ID NO: 1627 | uvig_81090 |
| SEQ ID NO: 80 | SEQ ID NO: 854 | SEQ ID NO: 1628 | uvig_227260 |
| SEQ ID NO: 81 | SEQ ID NO: 855 | SEQ ID NO: 1629 | uvig_581146 |
| SEQ ID NO: 82 | SEQ ID NO: 856 | SEQ ID NO: 1630 | uvig_64010 |
| SEQ ID NO: 83 | SEQ ID NO: 857 | SEQ ID NO: 1631 | uvig_87948 |
| SEQ ID NO: 84 | SEQ ID NO: 858 | SEQ ID NO: 1632 | uvig_392002 |
| SEQ ID NO: 85 | SEQ ID NO: 859 | SEQ ID NO: 1633 | uvig_229002 |
| SEQ ID NO: 86 | SEQ ID NO: 860 | SEQ ID NO: 1634 | uvig_548354 |
| SEQ ID NO: 87 | SEQ ID NO: 861 | SEQ ID NO: 1635 | uvig_100661 |
| SEQ ID NO: 88 | SEQ ID NO: 862 | SEQ ID NO: 1636 | uvig_107826 |
| SEQ ID NO: 89 | SEQ ID NO: 863 | SEQ ID NO: 1637 | uvig_254024 |
| SEQ ID NO: 90 | SEQ ID NO: 864 | SEQ ID NO: 1638 | uvig_182389 |
| SEQ ID NO: 91 | SEQ ID NO: 865 | SEQ ID NO: 1639 | uvig_102394 |
| SEQ ID NO: 92 | SEQ ID NO: 866 | SEQ ID NO: 1640 | uvig_585001 |
| SEQ ID NO: 93 | SEQ ID NO: 867 | SEQ ID NO: 1641 | uvig_255241 |
| SEQ ID NO: 94 | SEQ ID NO: 868 | SEQ ID NO: 1642 | uvig_376366 |
| SEQ ID NO: 95 | SEQ ID NO: 869 | SEQ ID NO: 1643 | uvig_6107 |
| SEQ ID NO: 96 | SEQ ID NO: 870 | SEQ ID NO: 1644 | uvig_368726 |
| SEQ ID NO: 97 | SEQ ID NO: 871 | SEQ ID NO: 1645 | uvig_206729 |
| SEQ ID NO: 98 | SEQ ID NO: 872 | SEQ ID NO: 1646 | uvig_539964 |
| SEQ ID NO: 99 | SEQ ID NO: 873 | SEQ ID NO: 1647 | uvig_70532 |
| SEQ ID NO: 100 | SEQ ID NO: 874 | SEQ ID NO: 1648 | uvig_418756 |
| SEQ ID NO: 101 | SEQ ID NO: 875 | SEQ ID NO: 1649 | uvig_187460 |
| SEQ ID NO: 102 | SEQ ID NO: 876 | SEQ ID NO: 1650 | uvig_441829 |
| SEQ ID NO: 103 | SEQ ID NO: 877 | SEQ ID NO: 1651 | uvig_517008 |
| SEQ ID NO: 104 | SEQ ID NO: 878 | SEQ ID NO: 1652 | uvig_368153 |
| SEQ ID NO: 105 | SEQ ID NO: 879 | SEQ ID NO: 1653 | uvig_310206 |
| SEQ ID NO: 106 | SEQ ID NO: 880 | SEQ ID NO: 1654 | uvig_541528 |
| SEQ ID NO: 107 | SEQ ID NO: 881 | SEQ ID NO: 1655 | uvig_539021 |
| SEQ ID NO: 108 | SEQ ID NO: 882 | SEQ ID NO: 1656 | uvig_467692 |
| SEQ ID NO: 109 | SEQ ID NO: 883 | SEQ ID NO: 1657 | uvig_188069 |
| SEQ ID NO: 110 | SEQ ID NO: 884 | SEQ ID NO: 1658 | uvig_138780 |
| SEQ ID NO: 111 | SEQ ID NO: 885 | SEQ ID NO: 1659 | uvig_588864 |
| SEQ ID NO: 112 | SEQ ID NO: 886 | SEQ ID NO: 1660 | uvig_150649 |
| SEQ ID NO: 113 | SEQ ID NO: 887 | SEQ ID NO: 1661 | uvig_473313 |
| SEQ ID NO: 114 | SEQ ID NO: 888 | SEQ ID NO: 1662 | uvig_192865 |
| SEQ ID NO: 115 | SEQ ID NO: 889 | SEQ ID NO: 1663 | uvig_444213 |
| SEQ ID NO: 116 | SEQ ID NO: 890 | SEQ ID NO: 1664 | uvig_223147 |
| SEQ ID NO: 117 | SEQ ID NO: 891 | SEQ ID NO: 1665 | uvig_80069 |
| SEQ ID NO: 118 | SEQ ID NO: 892 | SEQ ID NO: 1666 | uvig_594811 |
| SEQ ID NO: 119 | SEQ ID NO: 893 | SEQ ID NO: 1667 | uvig_239214 |
| SEQ ID NO: 120 | SEQ ID NO: 894 | SEQ ID NO: 1668 | uvig_65204 |
| SEQ ID NO: 121 | SEQ ID NO: 895 | SEQ ID NO: 1669 | uvig_597817 |
| SEQ ID NO: 122 | SEQ ID NO: 896 | SEQ ID NO: 1670 | uvig_35124 |
| SEQ ID NO: 123 | SEQ ID NO: 897 | SEQ ID NO: 1671 | uvig_550968 |
| SEQ ID NO: 124 | SEQ ID NO: 898 | SEQ ID NO: 1672 | uvig_296393 |
| SEQ ID NO: 125 | SEQ ID NO: 899 | SEQ ID NO: 1673 | uvig_311349 |
| SEQ ID NO: 126 | SEQ ID NO: 900 | SEQ ID NO: 1674 | uvig_245605 |
| SEQ ID NO: 127 | SEQ ID NO: 901 | SEQ ID NO: 1675 | uvig_163750 |
| SEQ ID NO: 128 | SEQ ID NO: 902 | SEQ ID NO: 1676 | uvig_75905 |
| SEQ ID NO: 129 | SEQ ID NO: 903 | SEQ ID NO: 1677 | uvig_151078 |
| SEQ ID NO: 130 | SEQ ID NO: 904 | SEQ ID NO: 1678 | uvig_195859 |
| SEQ ID NO: 131 | SEQ ID NO: 905 | SEQ ID NO: 1679 | uvig_150139 |
| SEQ ID NO: 132 | SEQ ID NO: 906 | SEQ ID NO: 1680 | uvig_224697 |
| SEQ ID NO: 133 | SEQ ID NO: 907 | SEQ ID NO: 1681 | uvig_395040 |
| SEQ ID NO: 134 | SEQ ID NO: 908 | SEQ ID NO: 1682 | uvig_581138 |
| SEQ ID NO: 135 | SEQ ID NO: 909 | SEQ ID NO: 1683 | uvig_225196 |
| SEQ ID NO: 136 | SEQ ID NO: 910 | SEQ ID NO: 1684 | uvig_230928 |
| SEQ ID NO: 137 | SEQ ID NO: 911 | SEQ ID NO: 1685 | uvig_197914 |
| SEQ ID NO: 138 | SEQ ID NO: 912 | SEQ ID NO: 1686 | uvig_148886 |
| SEQ ID NO: 139 | SEQ ID NO: 913 | SEQ ID NO: 1687 | uvig_106050 |
| SEQ ID NO: 140 | SEQ ID NO: 914 | SEQ ID NO: 1688 | uvig_203979 |
| SEQ ID NO: 141 | SEQ ID NO: 915 | SEQ ID NO: 1689 | uvig_75234 |
| SEQ ID NO: 142 | SEQ ID NO: 916 | SEQ ID NO: 1690 | uvig_180737 |
| SEQ ID NO: 143 | SEQ ID NO: 917 | SEQ ID NO: 1691 | uvig_354682 |
| SEQ ID NO: 144 | SEQ ID NO: 918 | SEQ ID NO: 1692 | uvig_292057 |
| SEQ ID NO: 145 | SEQ ID NO: 919 | SEQ ID NO: 1693 | uvig_181325 |
| SEQ ID NO: 146 | SEQ ID NO: 920 | SEQ ID NO: 1694 | uvig_568903 |
| SEQ ID NO: 147 | SEQ ID NO: 921 | SEQ ID NO: 1695 | uvig_254684 |
| SEQ ID NO: 148 | SEQ ID NO: 922 | SEQ ID NO: 1696 | uvig_368674 |
| SEQ ID NO: 149 | SEQ ID NO: 923 | SEQ ID NO: 1697 | uvig_538311 |
| SEQ ID NO: 150 | SEQ ID NO: 924 | SEQ ID NO: 1698 | uvig_131471 |
| SEQ ID NO: 151 | SEQ ID NO: 925 | SEQ ID NO: 1699 | uvig_435486 |
| SEQ ID NO: 152 | SEQ ID NO: 926 | SEQ ID NO: 1700 | uvig_136059 |
| SEQ ID NO: 153 | SEQ ID NO: 927 | SEQ ID NO: 1701 | uvig_279733 |
| SEQ ID NO: 154 | SEQ ID NO: 928 | SEQ ID NO: 1702 | uvig_58086 |
| SEQ ID NO: 155 | SEQ ID NO: 929 | SEQ ID NO: 1703 | uvig_564581 |
| SEQ ID NO: 156 | SEQ ID NO: 930 | SEQ ID NO: 1704 | ivig_3762 |
| SEQ ID NO: 157 | SEQ ID NO: 931 | SEQ ID NO: 1705 | uvig_580293 |
| SEQ ID NO: 158 | SEQ ID NO: 932 | SEQ ID NO: 1706 | uvig_584862 |
| SEQ ID NO: 159 | SEQ ID NO: 933 | SEQ ID NO: 1707 | uvig_195589 |
| SEQ ID NO: 160 | SEQ ID NO: 934 | SEQ ID NO: 1708 | uvig_27141 |
| SEQ ID NO: 161 | SEQ ID NO: 935 | SEQ ID NO: 1709 | uvig_173343 |
| SEQ ID NO: 162 | SEQ ID NO: 936 | SEQ ID NO: 1710 | uvig_9846 |
| SEQ ID NO: 163 | SEQ ID NO: 937 | SEQ ID NO: 1711 | uvig_278778 |
| SEQ ID NO: 164 | SEQ ID NO: 938 | SEQ ID NO: 1712 | uvig_178349 |
| SEQ ID NO: 165 | SEQ ID NO: 939 | SEQ ID NO: 1713 | uvig_286852 |
| SEQ ID NO: 166 | SEQ ID NO: 940 | SEQ ID NO: 1714 | uvig_452261 |
| SEQ ID NO: 167 | SEQ ID NO: 941 | SEQ ID NO: 1715 | uvig_70560 |
| SEQ ID NO: 168 | SEQ ID NO: 942 | SEQ ID NO: 1716 | ivig_2547 |
| SEQ ID NO: 169 | SEQ ID NO: 943 | SEQ ID NO: 1717 | uvig_443823 |
| SEQ ID NO: 170 | SEQ ID NO: 944 | SEQ ID NO: 1718 | uvig_268778 |
| SEQ ID NO: 171 | SEQ ID NO: 945 | SEQ ID NO: 1719 | uvig_579415 |
| SEQ ID NO: 172 | SEQ ID NO: 946 | SEQ ID NO: 1720 | uvig_465509 |
| SEQ ID NO: 173 | SEQ ID NO: 947 | SEQ ID NO: 1721 | uvig_539852 |
| SEQ ID NO: 174 | SEQ ID NO: 948 | SEQ ID NO: 1722 | ivig_4470 |
| SEQ ID NO: 175 | SEQ ID NO: 949 | SEQ ID NO: 1723 | uvig_590420 |
| SEQ ID NO: 176 | SEQ ID NO: 950 | SEQ ID NO: 1724 | uvig_271148 |
| SEQ ID NO: 177 | SEQ ID NO: 951 | SEQ ID NO: 1725 | uvig_373577 |
| SEQ ID NO: 178 | SEQ ID NO: 952 | SEQ ID NO: 1726 | uvig_217517 |
| SEQ ID NO: 179 | SEQ ID NO: 953 | SEQ ID NO: 1727 | uvig_581095 |
| SEQ ID NO: 180 | SEQ ID NO: 954 | SEQ ID NO: 1728 | QGJ85883.1 |
| SEQ ID NO: 181 | SEQ ID NO: 955 | SEQ ID NO: 1729 | uvig_460604 |
| SEQ ID NO: 182 | SEQ ID NO: 956 | SEQ ID NO: 1730 | uvig_200966 |
| SEQ ID NO: 183 | SEQ ID NO: 957 | SEQ ID NO: 1731 | uvig_540211 |
| SEQ ID NO: 184 | SEQ ID NO: 958 | SEQ ID NO: 1732 | uvig_533354 |
| SEQ ID NO: 185 | SEQ ID NO: 959 | SEQ ID NO: 1733 | uvig_424088 |
| SEQ ID NO: 186 | SEQ ID NO: 960 | SEQ ID NO: 1734 | uvig_334631 |
| SEQ ID NO: 187 | SEQ ID NO: 961 | SEQ ID NO: 1735 | uvig_80185 |
| SEQ ID NO: 188 | SEQ ID NO: 962 | SEQ ID NO: 1736 | uvig_538824 |
| SEQ ID NO: 189 | SEQ ID NO: 963 | SEQ ID NO: 1737 | uvig_390438 |
| SEQ ID NO: 190 | SEQ ID NO: 964 | SEQ ID NO: 1738 | uvig_91735 |
| SEQ ID NO: 191 | SEQ ID NO: 965 | SEQ ID NO: 1739 | uvig_188377 |
| SEQ ID NO: 192 | SEQ ID NO: 966 | SEQ ID NO: 1740 | uvig_192283 |
| SEQ ID NO: 193 | SEQ ID NO: 967 | SEQ ID NO: 1741 | uvig_180924 |
| SEQ ID NO: 194 | SEQ ID NO: 968 | SEQ ID NO: 1742 | uvig_129321 |
| SEQ ID NO: 195 | SEQ ID NO: 969 | SEQ ID NO: 1743 | uvig_127383 |
| SEQ ID NO: 196 | SEQ ID NO: 970 | SEQ ID NO: 1744 | uvig_184013 |
| SEQ ID NO: 197 | SEQ ID NO: 971 | SEQ ID NO: 1745 | uvig_538870 |
| SEQ ID NO: 198 | SEQ ID NO: 972 | SEQ ID NO: 1746 | uvig_254484 |
| SEQ ID NO: 199 | SEQ ID NO: 973 | SEQ ID NO: 1747 | uvig_295592 |
| SEQ ID NO: 200 | SEQ ID NO: 974 | SEQ ID NO: 1748 | uvig_280868 |
| SEQ ID NO: 201 | SEQ ID NO: 975 | SEQ ID NO: 1749 | uvig_428055 |
| SEQ ID NO: 202 | SEQ ID NO: 976 | SEQ ID NO: 1750 | uvig_565627 |
| SEQ ID NO: 203 | SEQ ID NO: 977 | SEQ ID NO: 1751 | uvig_118880 |
| SEQ ID NO: 204 | SEQ ID NO: 978 | SEQ ID NO: 1752 | uvig_124951 |
| SEQ ID NO: 205 | SEQ ID NO: 979 | SEQ ID NO: 1753 | uvig_295592 |
| SEQ ID NO: 206 | SEQ ID NO: 980 | SEQ ID NO: 1754 | uvig_30637 |
| SEQ ID NO: 207 | SEQ ID NO: 981 | SEQ ID NO: 1755 | uvig_230232 |
| SEQ ID NO: 208 | SEQ ID NO: 982 | SEQ ID NO: 1756 | uvig_220226 |
| SEQ ID NO: 209 | SEQ ID NO: 983 | SEQ ID NO: 1757 | uvig_229288 |
| SEQ ID NO: 210 | SEQ ID NO: 984 | SEQ ID NO: 1758 | uvig_51701 |
| SEQ ID NO: 211 | SEQ ID NO: 985 | SEQ ID NO: 1759 | uvig_254558 |
| SEQ ID NO: 212 | SEQ ID NO: 986 | SEQ ID NO: 1760 | ivig_3141 |
| SEQ ID NO: 213 | SEQ ID NO: 987 | SEQ ID NO: 1761 | uvig_123245 |
| SEQ ID NO: 214 | SEQ ID NO: 988 | SEQ ID NO: 1762 | uvig_138779 |
| SEQ ID NO: 215 | SEQ ID NO: 989 | SEQ ID NO: 1763 | uvig_22285 |
| SEQ ID NO: 216 | SEQ ID NO: 990 | SEQ ID NO: 1764 | uvig_67879 |
| SEQ ID NO: 217 | SEQ ID NO: 991 | SEQ ID NO: 1765 | uvig_505551 |
| SEQ ID NO: 218 | SEQ ID NO: 992 | SEQ ID NO: 1766 | uvig_433950 |
| SEQ ID NO: 219 | SEQ ID NO: 993 | SEQ ID NO: 1767 | uvig_123245 |
| SEQ ID NO: 220 | SEQ ID NO: 994 | SEQ ID NO: 1768 | uvig_205573 |
| SEQ ID NO: 221 | SEQ ID NO: 995 | SEQ ID NO: 1769 | uvig_311654 |
| SEQ ID NO: 222 | SEQ ID NO: 996 | SEQ ID NO: 1770 | uvig_268018 |
| SEQ ID NO: 223 | SEQ ID NO: 997 | SEQ ID NO: 1771 | uvig_490592 |
| SEQ ID NO: 224 | SEQ ID NO: 998 | SEQ ID NO: 1772 | uvig_220844 |
| SEQ ID NO: 225 | SEQ ID NO: 999 | SEQ ID NO: 1773 | uvig_127324 |
| SEQ ID NO: 226 | SEQ ID NO: 1000 | SEQ ID NO: 1774 | uvig_418530 |
| SEQ ID NO: 227 | SEQ ID NO: 1001 | SEQ ID NO: 1775 | uvig_169032 |
| SEQ ID NO: 228 | SEQ ID NO: 1002 | SEQ ID NO: 1776 | uvig_572866 |
| SEQ ID NO: 229 | SEQ ID NO: 1003 | SEQ ID NO: 1777 | uvig_327543 |
| SEQ ID NO: 230 | SEQ ID NO: 1004 | SEQ ID NO: 1778 | uvig_177600 |
| SEQ ID NO: 231 | SEQ ID NO: 1005 | SEQ ID NO: 1779 | uvig_457951 |
| SEQ ID NO: 232 | SEQ ID NO: 1006 | SEQ ID NO: 1780 | uvig_580269 |
| SEQ ID NO: 233 | SEQ ID NO: 1007 | SEQ ID NO: 1781 | uvig_576757 |
| SEQ ID NO: 234 | SEQ ID NO: 1008 | SEQ ID NO: 1782 | uvig_157917 |
| SEQ ID NO: 235 | SEQ ID NO: 1009 | SEQ ID NO: 1783 | uvig_555782 |
| SEQ ID NO: 236 | SEQ ID NO: 1010 | SEQ ID NO: 1784 | uvig_193710 |
| SEQ ID NO: 237 | SEQ ID NO: 1011 | SEQ ID NO: 1785 | uvig_363401 |
| SEQ ID NO: 238 | SEQ ID NO: 1012 | SEQ ID NO: 1786 | uvig_195006 |
| SEQ ID NO: 239 | SEQ ID NO: 1013 | SEQ ID NO: 1787 | uvig_180433 |
| SEQ ID NO: 240 | SEQ ID NO: 1014 | SEQ ID NO: 1788 | uvig_557335 |
| SEQ ID NO: 241 | SEQ ID NO: 1015 | SEQ ID NO: 1789 | uvig_89445 |
| SEQ ID NO: 242 | SEQ ID NO: 1016 | SEQ ID NO: 1790 | uvig_576812 |
| SEQ ID NO: 243 | SEQ ID NO: 1017 | SEQ ID NO: 1791 | uvig_1903 |
| SEQ ID NO: 244 | SEQ ID NO: 1018 | SEQ ID NO: 1792 | uvig_151346 |
| SEQ ID NO: 245 | SEQ ID NO: 1019 | SEQ ID NO: 1793 | uvig_282034 |
| SEQ ID NO: 246 | SEQ ID NO: 1020 | SEQ ID NO: 1794 | uvig_69193 |
| SEQ ID NO: 247 | SEQ ID NO: 1021 | SEQ ID NO: 1795 | uvig_318656 |
| SEQ ID NO: 248 | SEQ ID NO: 1022 | SEQ ID NO: 1796 | uvig_227965 |
| SEQ ID NO: 249 | SEQ ID NO: 1023 | SEQ ID NO: 1797 | uvig_182406 |
| SEQ ID NO: 250 | SEQ ID NO: 1024 | SEQ ID NO: 1798 | uvig_166254 |
| SEQ ID NO: 251 | SEQ ID NO: 1025 | SEQ ID NO: 1799 | uvig_586179 |
| SEQ ID NO: 252 | SEQ ID NO: 1026 | SEQ ID NO: 1800 | uvig_275384 |
| SEQ ID NO: 253 | SEQ ID NO: 1027 | SEQ ID NO: 1801 | uvig_128002 |
| SEQ ID NO: 254 | SEQ ID NO: 1028 | SEQ ID NO: 1802 | uvig_8240 |
| SEQ ID NO: 255 | SEQ ID NO: 1029 | SEQ ID NO: 1803 | uvig_86984 |
| SEQ ID NO: 256 | SEQ ID NO: 1030 | SEQ ID NO: 1804 | uvig_425758 |
| SEQ ID NO: 257 | SEQ ID NO: 1031 | SEQ ID NO: 1805 | uvig_158104 |
| SEQ ID NO: 258 | SEQ ID NO: 1032 | SEQ ID NO: 1806 | uvig_567103 |
| SEQ ID NO: 259 | SEQ ID NO: 1033 | SEQ ID NO: 1807 | uvig_58544 |
| SEQ ID NO: 260 | SEQ ID NO: 1034 | SEQ ID NO: 1808 | uvig_42250 |
| SEQ ID NO: 261 | SEQ ID NO: 1035 | SEQ ID NO: 1809 | uvig_198610 |
| SEQ ID NO: 262 | SEQ ID NO: 1036 | SEQ ID NO: 1810 | uvig_507007 |
| SEQ ID NO: 263 | SEQ ID NO: 1037 | SEQ ID NO: 1811 | uvig_232060 |
| SEQ ID NO: 264 | SEQ ID NO: 1038 | SEQ ID NO: 1812 | uvig_598290 |
| SEQ ID NO: 265 | SEQ ID NO: 1039 | SEQ ID NO: 1813 | uvig_73872 |
| SEQ ID NO: 266 | SEQ ID NO: 1040 | SEQ ID NO: 1814 | uvig_41106 |
| SEQ ID NO: 267 | SEQ ID NO: 1041 | SEQ ID NO: 1815 | uvig_53580 |
| SEQ ID NO: 268 | SEQ ID NO: 1042 | SEQ ID NO: 1816 | uvig_504803 |
| SEQ ID NO: 269 | SEQ ID NO: 1043 | SEQ ID NO: 1817 | uvig_198801 |
| SEQ ID NO: 270 | SEQ ID NO: 1044 | SEQ ID NO: 1818 | ivig_3837 |
| SEQ ID NO: 271 | SEQ ID NO: 1045 | SEQ ID NO: 1819 | uvig_58201 |
| SEQ ID NO: 272 | SEQ ID NO: 1046 | SEQ ID NO: 1820 | uvig_312745 |
| SEQ ID NO: 273 | SEQ ID NO: 1047 | SEQ ID NO: 1821 | uvig_287346 |
| SEQ ID NO: 274 | SEQ ID NO: 1048 | SEQ ID NO: 1822 | uvig_569915 |
| SEQ ID NO: 275 | SEQ ID NO: 1049 | SEQ ID NO: 1823 | uvig_380944 |
| SEQ ID NO: 276 | SEQ ID NO: 1050 | SEQ ID NO: 1824 | uvig_585281 |
| SEQ ID NO: 277 | SEQ ID NO: 1051 | SEQ ID NO: 1825 | uvig_85227 |
| SEQ ID NO: 278 | SEQ ID NO: 1052 | SEQ ID NO: 1826 | ivig_3177 |
| SEQ ID NO: 279 | SEQ ID NO: 1053 | SEQ ID NO: 1827 | uvig_83127 |
| SEQ ID NO: 280 | SEQ ID NO: 1054 | SEQ ID NO: 1828 | uvig_205904 |
| SEQ ID NO: 281 | SEQ ID NO: 1055 | SEQ ID NO: 1829 | uvig_239031 |
| SEQ ID NO: 282 | SEQ ID NO: 1056 | SEQ ID NO: 1830 | uvig_160559 |
| SEQ ID NO: 283 | SEQ ID NO: 1057 | SEQ ID NO: 1831 | uvig_135439 |
| SEQ ID NO: 284 | SEQ ID NO: 1058 | SEQ ID NO: 1832 | ivig_116 |
| SEQ ID NO: 285 | SEQ ID NO: 1059 | SEQ ID NO: 1833 | uvig_354027 |
| SEQ ID NO: 286 | SEQ ID NO: 1060 | SEQ ID NO: 1834 | uvig_305800 |
| SEQ ID NO: 287 | SEQ ID NO: 1061 | SEQ ID NO: 1835 | uvig_45 |
| SEQ ID NO: 288 | SEQ ID NO: 1062 | SEQ ID NO: 1836 | uvig_151078 |
| SEQ ID NO: 289 | SEQ ID NO: 1063 | SEQ ID NO: 1837 | uvig_369437 |
| SEQ ID NO: 290 | SEQ ID NO: 1064 | SEQ ID NO: 1838 | uvig_319734 |
| SEQ ID NO: 291 | SEQ ID NO: 1065 | SEQ ID NO: 1839 | uvig_183862 |
| SEQ ID NO: 292 | SEQ ID NO: 1066 | SEQ ID NO: 1840 | uvig_250794 |
| SEQ ID NO: 293 | SEQ ID NO: 1067 | SEQ ID NO: 1841 | uvig_195543 |
| SEQ ID NO: 294 | SEQ ID NO: 1068 | SEQ ID NO: 1842 | uvig_557325 |
| SEQ ID NO: 295 | SEQ ID NO: 1069 | SEQ ID NO: 1843 | uvig_13945 |
| SEQ ID NO: 296 | SEQ ID NO: 1070 | SEQ ID NO: 1844 | uvig_114897 |
| SEQ ID NO: 297 | SEQ ID NO: 1071 | SEQ ID NO: 1845 | uvig_505551 |
| SEQ ID NO: 298 | SEQ ID NO: 1072 | SEQ ID NO: 1846 | uvig_331196 |
| SEQ ID NO: 299 | SEQ ID NO: 1073 | SEQ ID NO: 1847 | uvig_80069 |
| SEQ ID NO: 300 | SEQ ID NO: 1074 | SEQ ID NO: 1848 | uvig_9192 |
| SEQ ID NO: 301 | SEQ ID NO: 1075 | SEQ ID NO: 1849 | uvig_507971 |
| SEQ ID NO: 302 | SEQ ID NO: 1076 | SEQ ID NO: 1850 | uvig_80003 |
| SEQ ID NO: 303 | SEQ ID NO: 1077 | SEQ ID NO: 1851 | uvig_176185 |
| SEQ ID NO: 304 | SEQ ID NO: 1078 | SEQ ID NO: 1852 | uvig_280693 |
| SEQ ID NO: 305 | SEQ ID NO: 1079 | SEQ ID NO: 1853 | uvig_81612 |
| SEQ ID NO: 306 | SEQ ID NO: 1080 | SEQ ID NO: 1854 | uvig_296980 |
| SEQ ID NO: 307 | SEQ ID NO: 1081 | SEQ ID NO: 1855 | uvig_517692 |
| SEQ ID NO: 308 | SEQ ID NO: 1082 | SEQ ID NO: 1856 | uvig_170697 |
| SEQ ID NO: 309 | SEQ ID NO: 1083 | SEQ ID NO: 1857 | uvig_55768 |
| SEQ ID NO: 310 | SEQ ID NO: 1084 | SEQ ID NO: 1858 | uvig_178167 |
| SEQ ID NO: 311 | SEQ ID NO: 1085 | SEQ ID NO: 1859 | uvig_66023 |
| SEQ ID NO: 312 | SEQ ID NO: 1086 | SEQ ID NO: 1860 | uvig_380785 |
| SEQ ID NO: 313 | SEQ ID NO: 1087 | SEQ ID NO: 1861 | uvig_388771 |
| SEQ ID NO: 314 | SEQ ID NO: 1088 | SEQ ID NO: 1862 | uvig_520747 |
| SEQ ID NO: 315 | SEQ ID NO: 1089 | SEQ ID NO: 1863 | uvig_380944 |
| SEQ ID NO: 316 | SEQ ID NO: 1090 | SEQ ID NO: 1864 | uvig_148875 |
| SEQ ID NO: 317 | SEQ ID NO: 1091 | SEQ ID NO: 1865 | uvig_143975 |
| SEQ ID NO: 318 | SEQ ID NO: 1092 | SEQ ID NO: 1866 | uvig_59951 |
| SEQ ID NO: 319 | SEQ ID NO: 1093 | SEQ ID NO: 1867 | uvig_368726 |
| SEQ ID NO: 320 | SEQ ID NO: 1094 | SEQ ID NO: 1868 | uvig_355166 |
| SEQ ID NO: 321 | SEQ ID NO: 1095 | SEQ ID NO: 1869 | uvig_345767 |
| SEQ ID NO: 322 | SEQ ID NO: 1096 | SEQ ID NO: 1870 | uvig_585265 |
| SEQ ID NO: 323 | SEQ ID NO: 1097 | SEQ ID NO: 1871 | ivig_2445 |
| SEQ ID NO: 324 | SEQ ID NO: 1098 | SEQ ID NO: 1872 | uvig_136571 |
| SEQ ID NO: 325 | SEQ ID NO: 1099 | SEQ ID NO: 1873 | uvig_469171 |
| SEQ ID NO: 326 | SEQ ID NO: 1100 | SEQ ID NO: 1874 | uvig_155463 |
| SEQ ID NO: 327 | SEQ ID NO: 1101 | SEQ ID NO: 1875 | uvig_597316 |
| SEQ ID NO: 328 | SEQ ID NO: 1102 | SEQ ID NO: 1876 | uvig_181332 |
| SEQ ID NO: 329 | SEQ ID NO: 1103 | SEQ ID NO: 1877 | uvig_222038 |
| SEQ ID NO: 330 | SEQ ID NO: 1104 | SEQ ID NO: 1878 | uvig_211114 |
| SEQ ID NO: 331 | SEQ ID NO: 1105 | SEQ ID NO: 1879 | uvig_473863 |
| SEQ ID NO: 332 | SEQ ID NO: 1106 | SEQ ID NO: 1880 | uvig_73839 |
| SEQ ID NO: 333 | SEQ ID NO: 1107 | SEQ ID NO: 1881 | uvig_154548 |
| SEQ ID NO: 334 | SEQ ID NO: 1108 | SEQ ID NO: 1882 | uvig_578394 |
| SEQ ID NO: 335 | SEQ ID NO: 1109 | SEQ ID NO: 1883 | uvig_175345 |
| SEQ ID NO: 336 | SEQ ID NO: 1110 | SEQ ID NO: 1884 | uvig_574139 |
| SEQ ID NO: 337 | SEQ ID NO: 1111 | SEQ ID NO: 1885 | uvig_189213 |
| SEQ ID NO: 338 | SEQ ID NO: 1112 | SEQ ID NO: 1886 | uvig_296776 |
| SEQ ID NO: 339 | SEQ ID NO: 1113 | SEQ ID NO: 1887 | uvig_173157 |
| SEQ ID NO: 340 | SEQ ID NO: 1114 | SEQ ID NO: 1888 | uvig_393561 |
| SEQ ID NO: 341 | SEQ ID NO: 1115 | SEQ ID NO: 1889 | uvig_296393 |
| SEQ ID NO: 342 | SEQ ID NO: 1116 | SEQ ID NO: 1890 | uvig_123245 |
| SEQ ID NO: 343 | SEQ ID NO: 1117 | SEQ ID NO: 1891 | uvig_58220 |
| SEQ ID NO: 344 | SEQ ID NO: 1118 | SEQ ID NO: 1892 | uvig_448547 |
| SEQ ID NO: 345 | SEQ ID NO: 1119 | SEQ ID NO: 1893 | uvig_400458 |
| SEQ ID NO: 346 | SEQ ID NO: 1120 | SEQ ID NO: 1894 | uvig_172639 |
| SEQ ID NO: 347 | SEQ ID NO: 1121 | SEQ ID NO: 1895 | uvig_189626 |
| SEQ ID NO: 348 | SEQ ID NO: 1122 | SEQ ID NO: 1896 | uvig_170915 |
| SEQ ID NO: 349 | SEQ ID NO: 1123 | SEQ ID NO: 1897 | uvig_195109 |
| SEQ ID NO: 350 | SEQ ID NO: 1124 | SEQ ID NO: 1898 | uvig_597743 |
| SEQ ID NO: 351 | SEQ ID NO: 1125 | SEQ ID NO: 1899 | uvig_595313 |
| SEQ ID NO: 352 | SEQ ID NO: 1126 | SEQ ID NO: 1900 | uvig_244256 |
| SEQ ID NO: 353 | SEQ ID NO: 1127 | SEQ ID NO: 1901 | uvig_555240 |
| SEQ ID NO: 354 | SEQ ID NO: 1128 | SEQ ID NO: 1902 | uvig_336926 |
| SEQ ID NO: 355 | SEQ ID NO: 1129 | SEQ ID NO: 1903 | uvig_239031 |
| SEQ ID NO: 356 | SEQ ID NO: 1130 | SEQ ID NO: 1904 | uvig_146316 |
| SEQ ID NO: 357 | SEQ ID NO: 1131 | SEQ ID NO: 1905 | uvig_390637 |
| SEQ ID NO: 358 | SEQ ID NO: 1132 | SEQ ID NO: 1906 | uvig_383825 |
| SEQ ID NO: 359 | SEQ ID NO: 1133 | SEQ ID NO: 1907 | ivig_4414 |
| SEQ ID NO: 360 | SEQ ID NO: 1134 | SEQ ID NO: 1908 | uvig_80069 |
| SEQ ID NO: 361 | SEQ ID NO: 1135 | SEQ ID NO: 1909 | uvig_395426 |
| SEQ ID NO: 362 | SEQ ID NO: 1136 | SEQ ID NO: 1910 | uvig_434714 |
| SEQ ID NO: 363 | SEQ ID NO: 1137 | SEQ ID NO: 1911 | uvig_425922 |
| SEQ ID NO: 364 | SEQ ID NO: 1138 | SEQ ID NO: 1912 | uvig_114951 |
| SEQ ID NO: 365 | SEQ ID NO: 1139 | SEQ ID NO: 1913 | uvig_442872 |
| SEQ ID NO: 366 | SEQ ID NO: 1140 | SEQ ID NO: 1914 | uvig_510225 |
| SEQ ID NO: 367 | SEQ ID NO: 1141 | SEQ ID NO: 1915 | uvig_281842 |
| SEQ ID NO: 368 | SEQ ID NO: 1142 | SEQ ID NO: 1916 | ivig_126 |
| SEQ ID NO: 369 | SEQ ID NO: 1143 | SEQ ID NO: 1917 | uvig_581976 |
| SEQ ID NO: 370 | SEQ ID NO: 1144 | SEQ ID NO: 1918 | uvig_49690 |
| SEQ ID NO: 371 | SEQ ID NO: 1145 | SEQ ID NO: 1919 | uvig_572170 |
| SEQ ID NO: 372 | SEQ ID NO: 1146 | SEQ ID NO: 1920 | ivig_2192 |
| SEQ ID NO: 373 | SEQ ID NO: 1147 | SEQ ID NO: 1921 | uvig_394091 |
| SEQ ID NO: 374 | SEQ ID NO: 1148 | SEQ ID NO: 1922 | uvig_598290 |
| SEQ ID NO: 375 | SEQ ID NO: 1149 | SEQ ID NO: 1923 | uvig_517008 |
| SEQ ID NO: 376 | SEQ ID NO: 1150 | SEQ ID NO: 1924 | uvig_223324 |
| SEQ ID NO: 377 | SEQ ID NO: 1151 | SEQ ID NO: 1925 | uvig_347727 |
| SEQ ID NO: 378 | SEQ ID NO: 1152 | SEQ ID NO: 1926 | uvig_47521 |
| SEQ ID NO: 379 | SEQ ID NO: 1153 | SEQ ID NO: 1927 | uvig_539751 |
| SEQ ID NO: 380 | SEQ ID NO: 1154 | SEQ ID NO: 1928 | uvig_124673 |
| SEQ ID NO: 381 | SEQ ID NO: 1155 | SEQ ID NO: 1929 | uvig_430255 |
| SEQ ID NO: 382 | SEQ ID NO: 1156 | SEQ ID NO: 1930 | uvig_581111 |
| SEQ ID NO: 383 | SEQ ID NO: 1157 | SEQ ID NO: 1931 | uvig_154343 |
| SEQ ID NO: 384 | SEQ ID NO: 1158 | SEQ ID NO: 1932 | uvig_597740 |
| SEQ ID NO: 385 | SEQ ID NO: 1159 | SEQ ID NO: 1933 | ivig_2971 |
| SEQ ID NO: 386 | SEQ ID NO: 1160 | SEQ ID NO: 1934 | uvig_458373 |
| SEQ ID NO: 387 | SEQ ID NO: 1161 | SEQ ID NO: 1935 | uvig_200526 |
| SEQ ID NO: 388 | SEQ ID NO: 1162 | SEQ ID NO: 1936 | uvig_307306 |
| SEQ ID NO: 389 | SEQ ID NO: 1163 | SEQ ID NO: 1937 | uvig_396131 |
| SEQ ID NO: 390 | SEQ ID NO: 1164 | SEQ ID NO: 1938 | uvig_568903 |
| SEQ ID NO: 391 | SEQ ID NO: 1165 | SEQ ID NO: 1939 | uvig_199869 |
| SEQ ID NO: 392 | SEQ ID NO: 1166 | SEQ ID NO: 1940 | uvig_181597 |
| SEQ ID NO: 393 | SEQ ID NO: 1167 | SEQ ID NO: 1941 | uvig_38906 |
| SEQ ID NO: 394 | SEQ ID NO: 1168 | SEQ ID NO: 1942 | uvig_16065 |
| SEQ ID NO: 395 | SEQ ID NO: 1169 | SEQ ID NO: 1943 | uvig_453325 |
| SEQ ID NO: 396 | SEQ ID NO: 1170 | SEQ ID NO: 1944 | uvig_294132 |
| SEQ ID NO: 397 | SEQ ID NO: 1171 | SEQ ID NO: 1945 | uvig_550213 |
| SEQ ID NO: 398 | SEQ ID NO: 1172 | SEQ ID NO: 1946 | uvig_442559 |
| SEQ ID NO: 399 | SEQ ID NO: 1173 | SEQ ID NO: 1947 | uvig_148886 |
| SEQ ID NO: 400 | SEQ ID NO: 1174 | SEQ ID NO: 1948 | ivig_4396 |
| SEQ ID NO: 401 | SEQ ID NO: 1175 | SEQ ID NO: 1949 | uvig_284398 |
| SEQ ID NO: 402 | SEQ ID NO: 1176 | SEQ ID NO: 1950 | uvig_517692 |
| SEQ ID NO: 403 | SEQ ID NO: 1177 | SEQ ID NO: 1951 | ivig_1929 |
| SEQ ID NO: 404 | SEQ ID NO: 1178 | SEQ ID NO: 1952 | uvig_35057 |
| SEQ ID NO: 405 | SEQ ID NO: 1179 | SEQ ID NO: 1953 | uvig_35057 |
| SEQ ID NO: 406 | SEQ ID NO: 1180 | SEQ ID NO: 1954 | uvig_174247 |
| SEQ ID NO: 407 | SEQ ID NO: 1181 | SEQ ID NO: 1955 | uvig_163358 |
| SEQ ID NO: 408 | SEQ ID NO: 1182 | SEQ ID NO: 1956 | ivig_2547 |
| SEQ ID NO: 409 | SEQ ID NO: 1183 | SEQ ID NO: 1957 | uvig_13765 |
| SEQ ID NO: 410 | SEQ ID NO: 1184 | SEQ ID NO: 1958 | uvig_151707 |
| SEQ ID NO: 411 | SEQ ID NO: 1185 | SEQ ID NO: 1959 | uvig_380829 |
| SEQ ID NO: 412 | SEQ ID NO: 1186 | SEQ ID NO: 1960 | uvig_83213 |
| SEQ ID NO: 413 | SEQ ID NO: 1187 | SEQ ID NO: 1961 | uvig_206323 |
| SEQ ID NO: 414 | SEQ ID NO: 1188 | SEQ ID NO: 1962 | uvig_404291 |
| SEQ ID NO: 415 | SEQ ID NO: 1189 | SEQ ID NO: 1963 | ivig_756 |
| SEQ ID NO: 416 | SEQ ID NO: 1190 | SEQ ID NO: 1964 | ivig_2327 |
| SEQ ID NO: 417 | SEQ ID NO: 1191 | SEQ ID NO: 1965 | uvig_554333 |
| SEQ ID NO: 418 | SEQ ID NO: 1192 | SEQ ID NO: 1966 | uvig_257872 |
| SEQ ID NO: 419 | SEQ ID NO: 1193 | SEQ ID NO: 1967 | uvig_210496 |
| SEQ ID NO: 420 | SEQ ID NO: 1194 | SEQ ID NO: 1968 | uvig_151237 |
| SEQ ID NO: 421 | SEQ ID NO: 1195 | SEQ ID NO: 1969 | uvig_206100 |
| SEQ ID NO: 422 | SEQ ID NO: 1196 | SEQ ID NO: 1970 | uvig_134660 |
| SEQ ID NO: 423 | SEQ ID NO: 1197 | SEQ ID NO: 1971 | uvig_234005 |
| SEQ ID NO: 424 | SEQ ID NO: 1198 | SEQ ID NO: 1972 | uvig_146622 |
| SEQ ID NO: 425 | SEQ ID NO: 1199 | SEQ ID NO: 1973 | uvig_356610 |
| SEQ ID NO: 426 | SEQ ID NO: 1200 | SEQ ID NO: 1974 | uvig_243310 |
| SEQ ID NO: 427 | SEQ ID NO: 1201 | SEQ ID NO: 1975 | uvig_278686 |
| SEQ ID NO: 428 | SEQ ID NO: 1202 | SEQ ID NO: 1976 | uvig_441833 |
| SEQ ID NO: 429 | SEQ ID NO: 1203 | SEQ ID NO: 1977 | uvig_584681 |
| SEQ ID NO: 430 | SEQ ID NO: 1204 | SEQ ID NO: 1978 | uvig_441567 |
| SEQ ID NO: 431 | SEQ ID NO: 1205 | SEQ ID NO: 1979 | uvig_3575 |
| SEQ ID NO: 432 | SEQ ID NO: 1206 | SEQ ID NO: 1980 | uvig_195822 |
| SEQ ID NO: 433 | SEQ ID NO: 1207 | SEQ ID NO: 1981 | uvig_386577 |
| SEQ ID NO: 434 | SEQ ID NO: 1208 | SEQ ID NO: 1982 | uvig_381373 |
| SEQ ID NO: 435 | SEQ ID NO: 1209 | SEQ ID NO: 1983 | uvig_100318 |
| SEQ ID NO: 436 | SEQ ID NO: 1210 | SEQ ID NO: 1984 | uvig_206650 |
| SEQ ID NO: 437 | SEQ ID NO: 1211 | SEQ ID NO: 1985 | uvig_192865 |
| SEQ ID NO: 438 | SEQ ID NO: 1212 | SEQ ID NO: 1986 | uvig_416748 |
| SEQ ID NO: 439 | SEQ ID NO: 1213 | SEQ ID NO: 1987 | uvig_495199 |
| SEQ ID NO: 440 | SEQ ID NO: 1214 | SEQ ID NO: 1988 | uvig_305979 |
| SEQ ID NO: 441 | SEQ ID NO: 1215 | SEQ ID NO: 1989 | uvig_291363 |
| SEQ ID NO: 442 | SEQ ID NO: 1216 | SEQ ID NO: 1990 | uvig_263829 |
| SEQ ID NO: 443 | SEQ ID NO: 1217 | SEQ ID NO: 1991 | uvig_13765 |
| SEQ ID NO: 444 | SEQ ID NO: 1218 | SEQ ID NO: 1992 | uvig_527169 |
| SEQ ID NO: 445 | SEQ ID NO: 1219 | SEQ ID NO: 1993 | uvig_133907 |
| SEQ ID NO: 446 | SEQ ID NO: 1220 | SEQ ID NO: 1994 | uvig_8523 |
| SEQ ID NO: 447 | SEQ ID NO: 1221 | SEQ ID NO: 1995 | uvig_361885 |
| SEQ ID NO: 448 | SEQ ID NO: 1222 | SEQ ID NO: 1996 | uvig_186102 |
| SEQ ID NO: 449 | SEQ ID NO: 1223 | SEQ ID NO: 1997 | uvig_183615 |
| SEQ ID NO: 450 | SEQ ID NO: 1224 | SEQ ID NO: 1998 | uvig_159029 |
| SEQ ID NO: 451 | SEQ ID NO: 1225 | SEQ ID NO: 1999 | uvig_89669 |
| SEQ ID NO: 452 | SEQ ID NO: 1226 | SEQ ID NO: 2000 | uvig_47505 |
| SEQ ID NO: 453 | SEQ ID NO: 1227 | SEQ ID NO: 2001 | uvig_51452 |
| SEQ ID NO: 454 | SEQ ID NO: 1228 | SEQ ID NO: 2002 | uvig_239031 |
| SEQ ID NO: 455 | SEQ ID NO: 1229 | SEQ ID NO: 2003 | uvig_543352 |
| SEQ ID NO: 456 | SEQ ID NO: 1230 | SEQ ID NO: 2004 | uvig_248716 |
| SEQ ID NO: 457 | SEQ ID NO: 1231 | SEQ ID NO: 2005 | uvig_366853 |
| SEQ ID NO: 458 | SEQ ID NO: 1232 | SEQ ID NO: 2006 | uvig_203185 |
| SEQ ID NO: 459 | SEQ ID NO: 1233 | SEQ ID NO: 2007 | uvig_54187 |
| SEQ ID NO: 460 | SEQ ID NO: 1234 | SEQ ID NO: 2008 | uvig_373913 |
| SEQ ID NO: 461 | SEQ ID NO: 1235 | SEQ ID NO: 2009 | uvig_284738 |
| SEQ ID NO: 462 | SEQ ID NO: 1236 | SEQ ID NO: 2010 | uvig_31017 |
| SEQ ID NO: 463 | SEQ ID NO: 1237 | SEQ ID NO: 2011 | uvig_51541 |
| SEQ ID NO: 464 | SEQ ID NO: 1238 | SEQ ID NO: 2012 | uvig_525361 |
| SEQ ID NO: 465 | SEQ ID NO: 1239 | SEQ ID NO: 2013 | uvig_520815 |
| SEQ ID NO: 466 | SEQ ID NO: 1240 | SEQ ID NO: 2014 | uvig_92124 |
| SEQ ID NO: 467 | SEQ ID NO: 1241 | SEQ ID NO: 2015 | uvig_588507 |
| SEQ ID NO: 468 | SEQ ID NO: 1242 | SEQ ID NO: 2016 | uvig_129895 |
| SEQ ID NO: 469 | SEQ ID NO: 1243 | SEQ ID NO: 2017 | uvig_74804 |
| SEQ ID NO: 470 | SEQ ID NO: 1244 | SEQ ID NO: 2018 | uvig_9192 |
| SEQ ID NO: 471 | SEQ ID NO: 1245 | SEQ ID NO: 2019 | uvig_190248 |
| SEQ ID NO: 472 | SEQ ID NO: 1246 | SEQ ID NO: 2020 | uvig_41106 |
| SEQ ID NO: 473 | SEQ ID NO: 1247 | SEQ ID NO: 2021 | uvig_453452 |
| SEQ ID NO: 474 | SEQ ID NO: 1248 | SEQ ID NO: 2022 | uvig_244564 |
| SEQ ID NO: 475 | SEQ ID NO: 1249 | SEQ ID NO: 2023 | uvig_563601 |
| SEQ ID NO: 476 | SEQ ID NO: 1250 | SEQ ID NO: 2024 | uvig_203635 |
| SEQ ID NO: 477 | SEQ ID NO: 1251 | SEQ ID NO: 2025 | uvig_311594 |
| SEQ ID NO: 478 | SEQ ID NO: 1252 | SEQ ID NO: 2026 | uvig_85018 |
| SEQ ID NO: 479 | SEQ ID NO: 1253 | SEQ ID NO: 2027 | uvig_81090 |
| SEQ ID NO: 480 | SEQ ID NO: 1254 | SEQ ID NO: 2028 | uvig_81430 |
| SEQ ID NO: 481 | SEQ ID NO: 1255 | SEQ ID NO: 2029 | uvig_144265 |
| SEQ ID NO: 482 | SEQ ID NO: 1256 | SEQ ID NO: 2030 | uvig_597427 |
| SEQ ID NO: 483 | SEQ ID NO: 1257 | SEQ ID NO: 2031 | uvig_593889 |
| SEQ ID NO: 484 | SEQ ID NO: 1258 | SEQ ID NO: 2032 | uvig_55768 |
| SEQ ID NO: 485 | SEQ ID NO: 1259 | SEQ ID NO: 2033 | uvig_120053 |
| SEQ ID NO: 486 | SEQ ID NO: 1260 | SEQ ID NO: 2034 | uvig_441833 |
| SEQ ID NO: 487 | SEQ ID NO: 1261 | SEQ ID NO: 2035 | uvig_210070 |
| SEQ ID NO: 488 | SEQ ID NO: 1262 | SEQ ID NO: 2036 | uvig_236827 |
| SEQ ID NO: 489 | SEQ ID NO: 1263 | SEQ ID NO: 2037 | uvig_393304 |
| SEQ ID NO: 490 | SEQ ID NO: 1264 | SEQ ID NO: 2038 | uvig_55768 |
| SEQ ID NO: 491 | SEQ ID NO: 1265 | SEQ ID NO: 2039 | uvig_227545 |
| SEQ ID NO: 492 | SEQ ID NO: 1266 | SEQ ID NO: 2040 | uvig_285944 |
| SEQ ID NO: 493 | SEQ ID NO: 1267 | SEQ ID NO: 2041 | uvig_224805 |
| SEQ ID NO: 494 | SEQ ID NO: 1268 | SEQ ID NO: 2042 | uvig_395773 |
| SEQ ID NO: 495 | SEQ ID NO: 1269 | SEQ ID NO: 2043 | ivig_749 |
| SEQ ID NO: 496 | SEQ ID NO: 1270 | SEQ ID NO: 2044 | uvig_537547 |
| SEQ ID NO: 497 | SEQ ID NO: 1271 | SEQ ID NO: 2045 | uvig_449731 |
| SEQ ID NO: 498 | SEQ ID NO: 1272 | SEQ ID NO: 2046 | uvig_287167 |
| SEQ ID NO: 499 | SEQ ID NO: 1273 | SEQ ID NO: 2047 | uvig_189213 |
| SEQ ID NO: 500 | SEQ ID NO: 1274 | SEQ ID NO: 2048 | uvig_437676 |
| SEQ ID NO: 501 | SEQ ID NO: 1275 | SEQ ID NO: 2049 | uvig_535546 |
| SEQ ID NO: 502 | SEQ ID NO: 1276 | SEQ ID NO: 2050 | uvig_102394 |
| SEQ ID NO: 503 | SEQ ID NO: 1277 | SEQ ID NO: 2051 | uvig_318842 |
| SEQ ID NO: 504 | SEQ ID NO: 1278 | SEQ ID NO: 2052 | uvig_284065 |
| SEQ ID NO: 505 | SEQ ID NO: 1279 | SEQ ID NO: 2053 | uvig_495062 |
| SEQ ID NO: 506 | SEQ ID NO: 1280 | SEQ ID NO: 2054 | uvig_151327 |
| SEQ ID NO: 507 | SEQ ID NO: 1281 | SEQ ID NO: 2055 | uvig_61202 |
| SEQ ID NO: 508 | SEQ ID NO: 1282 | SEQ ID NO: 2056 | uvig_393944 |
| SEQ ID NO: 509 | SEQ ID NO: 1283 | SEQ ID NO: 2057 | uvig_53595 |
| SEQ ID NO: 510 | SEQ ID NO: 1284 | SEQ ID NO: 2058 | uvig_342637 |
| SEQ ID NO: 511 | SEQ ID NO: 1285 | SEQ ID NO: 2059 | uvig_173210 |
| SEQ ID NO: 512 | SEQ ID NO: 1286 | SEQ ID NO: 2060 | uvig_13498 |
| SEQ ID NO: 513 | SEQ ID NO: 1287 | SEQ ID NO: 2061 | uvig_313242 |
| SEQ ID NO: 514 | SEQ ID NO: 1288 | SEQ ID NO: 2062 | uvig_212380 |
| SEQ ID NO: 515 | SEQ ID NO: 1289 | SEQ ID NO: 2063 | uvig_34482 |
| SEQ ID NO: 516 | SEQ ID NO: 1290 | SEQ ID NO: 2064 | uvig_463416 |
| SEQ ID NO: 517 | SEQ ID NO: 1291 | SEQ ID NO: 2065 | uvig_346035 |
| SEQ ID NO: 518 | SEQ ID NO: 1292 | SEQ ID NO: 2066 | uvig_375837 |
| SEQ ID NO: 519 | SEQ ID NO: 1293 | SEQ ID NO: 2067 | uvig_324806 |
| SEQ ID NO: 520 | SEQ ID NO: 1294 | SEQ ID NO: 2068 | uvig_527025 |
| SEQ ID NO: 521 | SEQ ID NO: 1295 | SEQ ID NO: 2069 | uvig_450121 |
| SEQ ID NO: 522 | SEQ ID NO: 1296 | SEQ ID NO: 2070 | uvig_42449 |
| SEQ ID NO: 523 | SEQ ID NO: 1297 | SEQ ID NO: 2071 | uvig_396773 |
| SEQ ID NO: 524 | SEQ ID NO: 1298 | SEQ ID NO: 2072 | ivig_4126 |
| SEQ ID NO: 525 | SEQ ID NO: 1299 | SEQ ID NO: 2073 | uvig_591587 |
| SEQ ID NO: 526 | SEQ ID NO: 1300 | SEQ ID NO: 2074 | uvig_39360 |
| SEQ ID NO: 527 | SEQ ID NO: 1301 | SEQ ID NO: 2075 | uvig_460722 |
| SEQ ID NO: 528 | SEQ ID NO: 1302 | SEQ ID NO: 2076 | uvig_288194 |
| SEQ ID NO: 529 | SEQ ID NO: 1303 | SEQ ID NO: 2077 | uvig_296879 |
| SEQ ID NO: 530 | SEQ ID NO: 1304 | SEQ ID NO: 2078 | uvig_151499 |
| SEQ ID NO: 531 | SEQ ID NO: 1305 | SEQ ID NO: 2079 | uvig_539135 |
| SEQ ID NO: 532 | SEQ ID NO: 1306 | SEQ ID NO: 2080 | uvig_57166 |
| SEQ ID NO: 533 | SEQ ID NO: 1307 | SEQ ID NO: 2081 | uvig_577393 |
| SEQ ID NO: 534 | SEQ ID NO: 1308 | SEQ ID NO: 2082 | uvig_365918 |
| SEQ ID NO: 535 | SEQ ID NO: 1309 | SEQ ID NO: 2083 | uvig_57063 |
| SEQ ID NO: 536 | SEQ ID NO: 1310 | SEQ ID NO: 2084 | uvig_586504 |
| SEQ ID NO: 537 | SEQ ID NO: 1311 | SEQ ID NO: 2085 | uvig_135914 |
| SEQ ID NO: 538 | SEQ ID NO: 1312 | SEQ ID NO: 2086 | uvig_256011 |
| SEQ ID NO: 539 | SEQ ID NO: 1313 | SEQ ID NO: 2087 | uvig_150631 |
| SEQ ID NO: 540 | SEQ ID NO: 1314 | SEQ ID NO: 2088 | uvig_541260 |
| SEQ ID NO: 541 | SEQ ID NO: 1315 | SEQ ID NO: 2089 | uvig_484218 |
| SEQ ID NO: 542 | SEQ ID NO: 1316 | SEQ ID NO: 2090 | uvig_287622 |
| SEQ ID NO: 543 | SEQ ID NO: 1317 | SEQ ID NO: 2091 | uvig_138265 |
| SEQ ID NO: 544 | SEQ ID NO: 1318 | SEQ ID NO: 2092 | uvig_378326 |
| SEQ ID NO: 545 | SEQ ID NO: 1319 | SEQ ID NO: 2093 | uvig_598266 |
| SEQ ID NO: 546 | SEQ ID NO: 1320 | SEQ ID NO: 2094 | uvig_289409 |
| SEQ ID NO: 547 | SEQ ID NO: 1321 | SEQ ID NO: 2095 | uvig_57389 |
| SEQ ID NO: 548 | SEQ ID NO: 1322 | SEQ ID NO: 2096 | uvig_25407 |
| SEQ ID NO: 549 | SEQ ID NO: 1323 | SEQ ID NO: 2097 | uvig_351737 |
| SEQ ID NO: 550 | SEQ ID NO: 1324 | SEQ ID NO: 2098 | uvig_155989 |
| SEQ ID NO: 551 | SEQ ID NO: 1325 | SEQ ID NO: 2099 | uvig_321891 |
| SEQ ID NO: 552 | SEQ ID NO: 1326 | SEQ ID NO: 2100 | uvig_151301 |
| SEQ ID NO: 553 | SEQ ID NO: 1327 | SEQ ID NO: 2101 | uvig_522525 |
| SEQ ID NO: 554 | SEQ ID NO: 1328 | SEQ ID NO: 2102 | uvig_517329 |
| SEQ ID NO: 555 | SEQ ID NO: 1329 | SEQ ID NO: 2103 | uvig_11457 |
| SEQ ID NO: 556 | SEQ ID NO: 1330 | SEQ ID NO: 2104 | uvig_285452 |
| SEQ ID NO: 557 | SEQ ID NO: 1331 | SEQ ID NO: 2105 | uvig_325705 |
| SEQ ID NO: 558 | SEQ ID NO: 1332 | SEQ ID NO: 2106 | uvig_205806 |
| SEQ ID NO: 559 | SEQ ID NO: 1333 | SEQ ID NO: 2107 | uvig_119010 |
| SEQ ID NO: 560 | SEQ ID NO: 1334 | SEQ ID NO: 2108 | uvig_115965 |
| SEQ ID NO: 561 | SEQ ID NO: 1335 | SEQ ID NO: 2109 | ivig_3513 |
| SEQ ID NO: 562 | SEQ ID NO: 1336 | SEQ ID NO: 2110 | uvig_598110 |
| SEQ ID NO: 563 | SEQ ID NO: 1337 | SEQ ID NO: 2111 | uvig_161644 |
| SEQ ID NO: 564 | SEQ ID NO: 1338 | SEQ ID NO: 2112 | uvig_116390 |
| SEQ ID NO: 565 | SEQ ID NO: 1339 | SEQ ID NO: 2113 | uvig_236553 |
| SEQ ID NO: 566 | SEQ ID NO: 1340 | SEQ ID NO: 2114 | uvig_370958 |
| SEQ ID NO: 567 | SEQ ID NO: 1341 | SEQ ID NO: 2115 | uvig_299740 |
| SEQ ID NO: 568 | SEQ ID NO: 1342 | SEQ ID NO: 2116 | ivig_1066 |
| SEQ ID NO: 569 | SEQ ID NO: 1343 | SEQ ID NO: 2117 | uvig_441476 |
| SEQ ID NO: 570 | SEQ ID NO: 1344 | SEQ ID NO: 2118 | uvig_112613 |
| SEQ ID NO: 571 | SEQ ID NO: 1345 | SEQ ID NO: 2119 | uvig_184056 |
| SEQ ID NO: 572 | SEQ ID NO: 1346 | SEQ ID NO: 2120 | uvig_111591 |
| SEQ ID NO: 573 | SEQ ID NO: 1347 | SEQ ID NO: 2121 | uvig_577010 |
| SEQ ID NO: 574 | SEQ ID NO: 1348 | SEQ ID NO: 2122 | uvig_476025 |
| SEQ ID NO: 575 | SEQ ID NO: 1349 | SEQ ID NO: 2123 | uvig_382772 |
| SEQ ID NO: 576 | SEQ ID NO: 1350 | SEQ ID NO: 2124 | uvig_512136 |
| SEQ ID NO: 577 | SEQ ID NO: 1351 | SEQ ID NO: 2125 | uvig_156529 |
| SEQ ID NO: 578 | SEQ ID NO: 1352 | SEQ ID NO: 2126 | uvig_594437 |
| SEQ ID NO: 579 | SEQ ID NO: 1353 | SEQ ID NO: 2127 | uvig_366074 |
| SEQ ID NO: 580 | SEQ ID NO: 1354 | SEQ ID NO: 2128 | uvig_573612 |
| SEQ ID NO: 581 | SEQ ID NO: 1355 | SEQ ID NO: 2129 | uvig_191392 |
| SEQ ID NO: 582 | SEQ ID NO: 1356 | SEQ ID NO: 2130 | uvig_587167 |
| SEQ ID NO: 583 | SEQ ID NO: 1357 | SEQ ID NO: 2131 | uvig_595287 |
| SEQ ID NO: 584 | SEQ ID NO: 1358 | SEQ ID NO: 2132 | uvig_329173 |
| SEQ ID NO: 585 | SEQ ID NO: 1359 | SEQ ID NO: 2133 | uvig_170733 |
| SEQ ID NO: 586 | SEQ ID NO: 1360 | SEQ ID NO: 2134 | uvig_400465 |
| SEQ ID NO: 587 | SEQ ID NO: 1361 | SEQ ID NO: 2135 | uvig_393882 |
| SEQ ID NO: 588 | SEQ ID NO: 1362 | SEQ ID NO: 2136 | uvig_587924 |
| SEQ ID NO: 589 | SEQ ID NO: 1363 | SEQ ID NO: 2137 | uvig_151182 |
| SEQ ID NO: 590 | SEQ ID NO: 1364 | SEQ ID NO: 2138 | uvig_383745 |
| SEQ ID NO: 591 | SEQ ID NO: 1365 | SEQ ID NO: 2139 | uvig_64089 |
| SEQ ID NO: 592 | SEQ ID NO: 1366 | SEQ ID NO: 2140 | uvig_563074 |
| SEQ ID NO: 593 | SEQ ID NO: 1367 | SEQ ID NO: 2141 | uvig_256936 |
| SEQ ID NO: 594 | SEQ ID NO: 1368 | SEQ ID NO: 2142 | uvig_110275 |
| SEQ ID NO: 595 | SEQ ID NO: 1369 | SEQ ID NO: 2143 | uvig_239325 |
| SEQ ID NO: 596 | SEQ ID NO: 1370 | SEQ ID NO: 2144 | uvig_578984 |
| SEQ ID NO: 597 | SEQ ID NO: 1371 | SEQ ID NO: 2145 | uvig_316826 |
| SEQ ID NO: 598 | SEQ ID NO: 1372 | SEQ ID NO: 2146 | uvig_86231 |
| SEQ ID NO: 599 | SEQ ID NO: 1373 | SEQ ID NO: 2147 | uvig_125074 |
| SEQ ID NO: 600 | SEQ ID NO: 1374 | SEQ ID NO: 2148 | uvig_337673 |
| SEQ ID NO: 601 | SEQ ID NO: 1375 | SEQ ID NO: 2149 | uvig_595969 |
| SEQ ID NO: 602 | SEQ ID NO: 1376 | SEQ ID NO: 2150 | uvig_177087 |
| SEQ ID NO: 603 | SEQ ID NO: 1377 | SEQ ID NO: 2151 | uvig_594539 |
| SEQ ID NO: 604 | SEQ ID NO: 1378 | SEQ ID NO: 2152 | uvig_236070 |
| SEQ ID NO: 605 | SEQ ID NO: 1379 | SEQ ID NO: 2153 | uvig_171405 |
| SEQ ID NO: 606 | SEQ ID NO: 1380 | SEQ ID NO: 2154 | uvig_578207 |
| SEQ ID NO: 607 | SEQ ID NO: 1381 | SEQ ID NO: 2155 | uvig_354904 |
| SEQ ID NO: 608 | SEQ ID NO: 1382 | SEQ ID NO: 2156 | uvig_15514 |
| SEQ ID NO: 609 | SEQ ID NO: 1383 | SEQ ID NO: 2157 | uvig_83898 |
| SEQ ID NO: 610 | SEQ ID NO: 1384 | SEQ ID NO: 2158 | uvig_246969 |
| SEQ ID NO: 611 | SEQ ID NO: 1385 | SEQ ID NO: 2159 | uvig_187146 |
| SEQ ID NO: 612 | SEQ ID NO: 1386 | SEQ ID NO: 2160 | uvig_132785 |
| SEQ ID NO: 613 | SEQ ID NO: 1387 | SEQ ID NO: 2161 | uvig_293930 |
| SEQ ID NO: 614 | SEQ ID NO: 1388 | SEQ ID NO: 2162 | uvig_306774 |
| SEQ ID NO: 615 | SEQ ID NO: 1389 | SEQ ID NO: 2163 | uvig_368839 |
| SEQ ID NO: 616 | SEQ ID NO: 1390 | SEQ ID NO: 2164 | uvig_105444 |
| SEQ ID NO: 617 | SEQ ID NO: 1391 | SEQ ID NO: 2165 | uvig_381374 |
| SEQ ID NO: 618 | SEQ ID NO: 1392 | SEQ ID NO: 2166 | uvig_330914 |
| SEQ ID NO: 619 | SEQ ID NO: 1393 | SEQ ID NO: 2167 | uvig_394534 |
| SEQ ID NO: 620 | SEQ ID NO: 1394 | SEQ ID NO: 2168 | uvig_582769 |
| SEQ ID NO: 621 | SEQ ID NO: 1395 | SEQ ID NO: 2169 | uvig_578663 |
| SEQ ID NO: 622 | SEQ ID NO: 1396 | SEQ ID NO: 2170 | uvig_103894 |
| SEQ ID NO: 623 | SEQ ID NO: 1397 | SEQ ID NO: 2171 | uvig_263922 |
| SEQ ID NO: 624 | SEQ ID NO: 1398 | SEQ ID NO: 2172 | uvig_156514 |
| SEQ ID NO: 625 | SEQ ID NO: 1399 | SEQ ID NO: 2173 | uvig_454524 |
| SEQ ID NO: 626 | SEQ ID NO: 1400 | SEQ ID NO: 2174 | uvig_204816 |
| SEQ ID NO: 627 | SEQ ID NO: 1401 | SEQ ID NO: 2175 | uvig_396721 |
| SEQ ID NO: 628 | SEQ ID NO: 1402 | SEQ ID NO: 2176 | uvig_593897 |
| SEQ ID NO: 629 | SEQ ID NO: 1403 | SEQ ID NO: 2177 | uvig_440207 |
| SEQ ID NO: 630 | SEQ ID NO: 1404 | SEQ ID NO: 2178 | uvig_578394 |
| SEQ ID NO: 631 | SEQ ID NO: 1405 | SEQ ID NO: 2179 | uvig_370045 |
| SEQ ID NO: 632 | SEQ ID NO: 1406 | SEQ ID NO: 2180 | uvig_93245 |
| SEQ ID NO: 633 | SEQ ID NO: 1407 | SEQ ID NO: 2181 | uvig_151615 |
| SEQ ID NO: 634 | SEQ ID NO: 1408 | SEQ ID NO: 2182 | uvig_327878 |
| SEQ ID NO: 635 | SEQ ID NO: 1409 | SEQ ID NO: 2183 | uvig_176611 |
| SEQ ID NO: 636 | SEQ ID NO: 1410 | SEQ ID NO: 2184 | uvig_154256 |
| SEQ ID NO: 637 | SEQ ID NO: 1411 | SEQ ID NO: 2185 | uvig_596302 |
| SEQ ID NO: 638 | SEQ ID NO: 1412 | SEQ ID NO: 2186 | uvig_118876 |
| SEQ ID NO: 639 | SEQ ID NO: 1413 | SEQ ID NO: 2187 | uvig_375705 |
| SEQ ID NO: 640 | SEQ ID NO: 1414 | SEQ ID NO: 2188 | QGJ86143.1 |
| SEQ ID NO: 641 | SEQ ID NO: 1415 | SEQ ID NO: 2189 | MT658802 |
| SEQ ID NO: 642 | SEQ ID NO: 1416 | SEQ ID NO: 2190 | QGJ86433.1 |
| SEQ ID NO: 643 | SEQ ID NO: 1417 | SEQ ID NO: 2191 | uvig_582533 |
| SEQ ID NO: 644 | SEQ ID NO: 1418 | SEQ ID NO: 2192 | QGJ85967.1 |
| SEQ ID NO: 645 | SEQ ID NO: 1419 | SEQ ID NO: 2193 | MZ322017 |
| SEQ ID NO: 646 | SEQ ID NO: 1420 | SEQ ID NO: 2194 | MW584159 |
| SEQ ID NO: 647 | SEQ ID NO: 1421 | SEQ ID NO: 2195 | CP063968 |
| SEQ ID NO: 648 | SEQ ID NO: 1422 | SEQ ID NO: 2196 | uvig_364726 |
| SEQ ID NO: 649 | SEQ ID NO: 1423 | SEQ ID NO: 2197 | uvig_118757 |
| SEQ ID NO: 650 | SEQ ID NO: 1424 | SEQ ID NO: 2198 | uvig_442496 |
| SEQ ID NO: 651 | SEQ ID NO: 1425 | SEQ ID NO: 2199 | uvig_425122 |
| SEQ ID NO: 652 | SEQ ID NO: 1426 | SEQ ID NO: 2200 | uvig_151019 |
| SEQ ID NO: 653 | SEQ ID NO: 1427 | SEQ ID NO: 2201 | uvig_570177 |
| SEQ ID NO: 654 | SEQ ID NO: 1428 | SEQ ID NO: 2202 | HQ906663 |
| SEQ ID NO: 655 | SEQ ID NO: 1429 | SEQ ID NO: 2203 | CP017837 |
| SEQ ID NO: 656 | SEQ ID NO: 1430 | SEQ ID NO: 2204 | MZ308445 |
| SEQ ID NO: 657 | SEQ ID NO: 1431 | SEQ ID NO: 2205 | MN585974 |
| SEQ ID NO: 658 | SEQ ID NO: 1432 | SEQ ID NO: 2206 | uvig_323824 |
| SEQ ID NO: 659 | SEQ ID NO: 1433 | SEQ ID NO: 2207 | uvig_314864 |
| SEQ ID NO: 660 | SEQ ID NO: 1434 | SEQ ID NO: 2208 | uvig_31244 |
| SEQ ID NO: 661 | SEQ ID NO: 1435 | SEQ ID NO: 2209 | uvig_328850 |
| SEQ ID NO: 662 | SEQ ID NO: 1436 | SEQ ID NO: 2210 | uvig_323858 |
| SEQ ID NO: 663 | SEQ ID NO: 1437 | SEQ ID NO: 2211 | uvig_520818 |
| SEQ ID NO: 664 | SEQ ID NO: 1438 | SEQ ID NO: 2212 | uvig_199031 |
| SEQ ID NO: 665 | SEQ ID NO: 1439 | SEQ ID NO: 2213 | uvig_17362 |
| SEQ ID NO: 666 | SEQ ID NO: 1440 | SEQ ID NO: 2214 | uvig_135797 |
| SEQ ID NO: 667 | SEQ ID NO: 1441 | SEQ ID NO: 2215 | uvig_240645 |
| SEQ ID NO: 668 | SEQ ID NO: 1442 | SEQ ID NO: 2216 | uvig_358290 |
| SEQ ID NO: 669 | SEQ ID NO: 1443 | SEQ ID NO: 2217 | uvig_357839 |
| SEQ ID NO: 670 | SEQ ID NO: 1444 | SEQ ID NO: 2218 | uvig_263250 |
| SEQ ID NO: 671 | SEQ ID NO: 1445 | SEQ ID NO: 2219 | uvig_148588 |
| SEQ ID NO: 672 | SEQ ID NO: 1446 | SEQ ID NO: 2220 | uvig_171237 |
| SEQ ID NO: 673 | SEQ ID NO: 1447 | SEQ ID NO: 2221 | ivig_3933 |
| SEQ ID NO: 674 | SEQ ID NO: 1448 | SEQ ID NO: 2222 | uvig_584312 |
| SEQ ID NO: 675 | SEQ ID NO: 1449 | SEQ ID NO: 2223 | uvig_80961 |
| SEQ ID NO: 676 | SEQ ID NO: 1450 | SEQ ID NO: 2224 | uvig_10984 |
| SEQ ID NO: 677 | SEQ ID NO: 1451 | SEQ ID NO: 2225 | uvig_226352 |
| SEQ ID NO: 678 | SEQ ID NO: 1452 | SEQ ID NO: 2226 | uvig_143228 |
| SEQ ID NO: 679 | SEQ ID NO: 1453 | SEQ ID NO: 2227 | uvig_579072 |
| SEQ ID NO: 680 | SEQ ID NO: 1454 | SEQ ID NO: 2228 | uvig_596872 |
| SEQ ID NO: 681 | SEQ ID NO: 1455 | SEQ ID NO: 2229 | uvig_381385 |
| SEQ ID NO: 682 | SEQ ID NO: 1456 | SEQ ID NO: 2230 | uvig_146439 |
| SEQ ID NO: 683 | SEQ ID NO: 1457 | SEQ ID NO: 2231 | uvig_423324 |
| SEQ ID NO: 684 | SEQ ID NO: 1458 | SEQ ID NO: 2232 | uvig_441018 |
| SEQ ID NO: 685 | SEQ ID NO: 1459 | SEQ ID NO: 2233 | uvig_426061 |
| SEQ ID NO: 686 | SEQ ID NO: 1460 | SEQ ID NO: 2234 | uvig_287690 |
| SEQ ID NO: 687 | SEQ ID NO: 1461 | SEQ ID NO: 2235 | uvig_61588 |
| SEQ ID NO: 688 | SEQ ID NO: 1462 | SEQ ID NO: 2236 | ivig_3872 |
| SEQ ID NO: 689 | SEQ ID NO: 1463 | SEQ ID NO: 2237 | uvig_541020 |
| SEQ ID NO: 690 | SEQ ID NO: 1464 | SEQ ID NO: 2238 | uvig_396371 |
| SEQ ID NO: 691 | SEQ ID NO: 1465 | SEQ ID NO: 2239 | uvig_301458 |
| SEQ ID NO: 692 | SEQ ID NO: 1466 | SEQ ID NO: 2240 | uvig_430479 |
| SEQ ID NO: 693 | SEQ ID NO: 1467 | SEQ ID NO: 2241 | uvig_425764 |
| SEQ ID NO: 694 | SEQ ID NO: 1468 | SEQ ID NO: 2242 | uvig_128102 |
| SEQ ID NO: 695 | SEQ ID NO: 1469 | SEQ ID NO: 2243 | uvig_294201 |
| SEQ ID NO: 696 | SEQ ID NO: 1470 | SEQ ID NO: 2244 | uvig_174822 |
| SEQ ID NO: 697 | SEQ ID NO: 1471 | SEQ ID NO: 2245 | ivig_1533 |
| SEQ ID NO: 698 | SEQ ID NO: 1472 | SEQ ID NO: 2246 | uvig_317982 |
| SEQ ID NO: 699 | SEQ ID NO: 1473 | SEQ ID NO: 2247 | uvig_598484 |
| SEQ ID NO: 700 | SEQ ID NO: 1474 | SEQ ID NO: 2248 | uvig_434341 |
| SEQ ID NO: 701 | SEQ ID NO: 1475 | SEQ ID NO: 2249 | uvig_323835 |
| SEQ ID NO: 702 | SEQ ID NO: 1476 | SEQ ID NO: 2250 | uvig_400028 |
| SEQ ID NO: 703 | SEQ ID NO: 1477 | SEQ ID NO: 2251 | uvig_100684 |
| SEQ ID NO: 704 | SEQ ID NO: 1478 | SEQ ID NO: 2252 | uvig_95947 |
| SEQ ID NO: 705 | SEQ ID NO: 1479 | SEQ ID NO: 2253 | uvig_392101 |
| SEQ ID NO: 706 | SEQ ID NO: 1480 | SEQ ID NO: 2254 | uvig_208975 |
| SEQ ID NO: 707 | SEQ ID NO: 1481 | SEQ ID NO: 2255 | uvig_586184 |
| SEQ ID NO: 708 | SEQ ID NO: 1482 | SEQ ID NO: 2256 | uvig_22576 |
| SEQ ID NO: 709 | SEQ ID NO: 1483 | SEQ ID NO: 2257 | uvig_581097 |
| SEQ ID NO: 710 | SEQ ID NO: 1484 | SEQ ID NO: 2258 | uvig_483710 |
| SEQ ID NO: 711 | SEQ ID NO: 1485 | SEQ ID NO: 2259 | uvig_255651 |
| SEQ ID NO: 712 | SEQ ID NO: 1486 | SEQ ID NO: 2260 | uvig_453602 |
| SEQ ID NO: 713 | SEQ ID NO: 1487 | SEQ ID NO: 2261 | uvig_370654 |
| SEQ ID NO: 714 | SEQ ID NO: 1488 | SEQ ID NO: 2262 | uvig_208980 |
| SEQ ID NO: 715 | SEQ ID NO: 1489 | SEQ ID NO: 2263 | uvig_127373 |
| SEQ ID NO: 716 | SEQ ID NO: 1490 | SEQ ID NO: 2264 | uvig_311977 |
| SEQ ID NO: 717 | SEQ ID NO: 1491 | SEQ ID NO: 2265 | uvig_349522 |
| SEQ ID NO: 718 | SEQ ID NO: 1492 | SEQ ID NO: 2266 | uvig_53024 |
| SEQ ID NO: 719 | SEQ ID NO: 1493 | SEQ ID NO: 2267 | uvig_595447 |
| SEQ ID NO: 720 | SEQ ID NO: 1494 | SEQ ID NO: 2268 | uvig_231300 |
| SEQ ID NO: 721 | SEQ ID NO: 1495 | SEQ ID NO: 2269 | uvig_476161 |
| SEQ ID NO: 722 | SEQ ID NO: 1496 | SEQ ID NO: 2270 | uvig_590668 |
| SEQ ID NO: 723 | SEQ ID NO: 1497 | SEQ ID NO: 2271 | uvig_150568 |
| SEQ ID NO: 724 | SEQ ID NO: 1498 | SEQ ID NO: 2272 | uvig_76620 |
| SEQ ID NO: 725 | SEQ ID NO: 1499 | SEQ ID NO: 2273 | uvig_419578 |
| SEQ ID NO: 726 | SEQ ID NO: 1500 | SEQ ID NO: 2274 | uvig_282819 |
| SEQ ID NO: 727 | SEQ ID NO: 1501 | SEQ ID NO: 2275 | uvig_577253 |
| SEQ ID NO: 728 | SEQ ID NO: 1502 | SEQ ID NO: 2276 | uvig_257578 |
| SEQ ID NO: 729 | SEQ ID NO: 1503 | SEQ ID NO: 2277 | uvig_437230 |
| SEQ ID NO: 730 | SEQ ID NO: 1504 | SEQ ID NO: 2278 | uvig_594175 |
| SEQ ID NO: 731 | SEQ ID NO: 1505 | SEQ ID NO: 2279 | uvig_593397 |
| SEQ ID NO: 732 | SEQ ID NO: 1506 | SEQ ID NO: 2280 | uvig_225515 |
| SEQ ID NO: 733 | SEQ ID NO: 1507 | SEQ ID NO: 2281 | uvig_107724 |
| SEQ ID NO: 734 | SEQ ID NO: 1508 | SEQ ID NO: 2282 | uvig_286002 |
| SEQ ID NO: 735 | SEQ ID NO: 1509 | SEQ ID NO: 2283 | uvig_25355 |
| SEQ ID NO: 736 | SEQ ID NO: 1510 | SEQ ID NO: 2284 | uvig_457901 |
| SEQ ID NO: 737 | SEQ ID NO: 1511 | SEQ ID NO: 2285 | uvig_247278 |
| SEQ ID NO: 738 | SEQ ID NO: 1512 | SEQ ID NO: 2286 | uvig_374979 |
| SEQ ID NO: 739 | SEQ ID NO: 1513 | SEQ ID NO: 2287 | uvig_140430 |
| SEQ ID NO: 740 | SEQ ID NO: 1514 | SEQ ID NO: 2288 | uvig_249187 |
| SEQ ID NO: 741 | SEQ ID NO: 1515 | SEQ ID NO: 2289 | uvig_199462 |
| SEQ ID NO: 742 | SEQ ID NO: 1516 | SEQ ID NO: 2290 | uvig_104410 |
| SEQ ID NO: 743 | SEQ ID NO: 1517 | SEQ ID NO: 2291 | uvig_324974 |
| SEQ ID NO: 744 | SEQ ID NO: 1518 | SEQ ID NO: 2292 | uvig_214087 |
| SEQ ID NO: 745 | SEQ ID NO: 1519 | SEQ ID NO: 2293 | uvig_13945 |
| SEQ ID NO: 746 | SEQ ID NO: 1520 | SEQ ID NO: 2294 | uvig_11401 |
| SEQ ID NO: 747 | SEQ ID NO: 1521 | SEQ ID NO: 2295 | uvig_81430 |
| SEQ ID NO: 748 | SEQ ID NO: 1522 | SEQ ID NO: 2296 | uvig_250870 |
| SEQ ID NO: 749 | SEQ ID NO: 1523 | SEQ ID NO: 2297 | uvig_590864 |
| SEQ ID NO: 750 | SEQ ID NO: 1524 | SEQ ID NO: 2298 | uvig_135439 |
| SEQ ID NO: 751 | SEQ ID NO: 1525 | SEQ ID NO: 2299 | uvig_166254 |
| SEQ ID NO: 752 | SEQ ID NO: 1526 | SEQ ID NO: 2300 | uvig_422831 |
| SEQ ID NO: 753 | SEQ ID NO: 1527 | SEQ ID NO: 2301 | ivig_3102 |
| SEQ ID NO: 754 | SEQ ID NO: 1528 | SEQ ID NO: 2302 | uvig_404379 |
| SEQ ID NO: 755 | SEQ ID NO: 1529 | SEQ ID NO: 2303 | uvig_554169 |
| SEQ ID NO: 756 | SEQ ID NO: 1530 | SEQ ID NO: 2304 | uvig_173267 |
| SEQ ID NO: 757 | SEQ ID NO: 1531 | SEQ ID NO: 2305 | uvig_110260 |
| SEQ ID NO: 758 | SEQ ID NO: 1532 | SEQ ID NO: 2306 | ivig_1400 |
| SEQ ID NO: 759 | SEQ ID NO: 1533 | SEQ ID NO: 2307 | uvig_144279 |
| SEQ ID NO: 760 | SEQ ID NO: 1534 | SEQ ID NO: 2308 | uvig_193710 |
| SEQ ID NO: 761 | SEQ ID NO: 1535 | SEQ ID NO: 2309 | uvig_256500 |
| SEQ ID NO: 762 | SEQ ID NO: 1536 | SEQ ID NO: 2310 | uvig_206777 |
| SEQ ID NO: 763 | SEQ ID NO: 1537 | SEQ ID NO: 2311 | uvig_158624 |
| SEQ ID NO: 764 | SEQ ID NO: 1538 | SEQ ID NO: 2312 | uvig_46185 |
| SEQ ID NO: 765 | SEQ ID NO: 1539 | SEQ ID NO: 2313 | uvig_593892 |
| SEQ ID NO: 766 | SEQ ID NO: 1540 | SEQ ID NO: 2314 | uvig_36383 |
| SEQ ID NO: 767 | SEQ ID NO: 1541 | SEQ ID NO: 2315 | uvig_384338 |
| SEQ ID NO: 768 | SEQ ID NO: 1542 | SEQ ID NO: 2316 | uvig_329211 |
| SEQ ID NO: 769 | SEQ ID NO: 1543 | SEQ ID NO: 2317 | uvig_163634 |
| SEQ ID NO: 770 | SEQ ID NO: 1544 | SEQ ID NO: 2318 | uvig_351740 |
| SEQ ID NO: 771 | SEQ ID NO: 1545 | SEQ ID NO: 2319 | QGJ86668.1 |
| SEQ ID NO: 772 | SEQ ID NO: 1546 | SEQ ID NO: 2320 | uvig_587893 |
| SEQ ID NO: 773 | SEQ ID NO: 1547 | SEQ ID NO: 2321 | uvig_195542 |
| SEQ ID NO: 774 | SEQ ID NO: 1548 | SEQ ID NO: 2322 | uvig_40909 |

### Heterologous Nucleic Acids

A large serine recombinase can mediate an integration of a heterologous nucleic acid molecule into the specific site in the target genome via the attP-attB complex. The heterologous nucleic acid can be a DNA molecule, RNA molecule, oligonucleotide, which is single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases either deoxyribonucleotides, ribonucleotides, or analogs thereof. heterologous nucleic acid molecules may have three-dimensional structure, may include coding or non-coding regions, may include exons, introns, mRNA, tRNA, rRNA, siRNA, shRNA, miRNA, ribozymes, cDNA, plasmids, vectors, exogenous sequences, endogenous sequences. A heterologous nucleic acid nucleic acid can comprise modified nucleotides, include methylated nucleotides, or nucleotide analogs. In some embodiments, a heterologous nucleic acid may be interspersed with non-nucleic acid components.

In some embodiments, the heterologous nucleic acid molecule may contain an open reading frame encoding a polypeptide of in heterologous nucleic acid molecule comprises a Kozak sequence, an internal ribosome entry site, a start codon, a stop codon, one or more exons, and one or more introns. In some embodiments, the heterologous nucleic acid molecule comprises a splice acceptor site, and/or a splice donor site. In some embodiments, the heterologous nucleic acid molecule comprises a 3′ UTR region, a 5′ UTR region, a microRNA binding site, a microRNA sequence, a siRNA sequence, a guide RNA sequence, a piwi RNA sequence, a poly(A) tail, e.g., downstream of the stop codon of an open reading frame. In some embodiments, the heterologous nucleic acid molecule comprises a promoter (e.g., constitute or inducible promoter), a eukaryotic transcriptional terminator, one or more translation enhancing elements. In some embodiments the promoter is an RNA polymerase I promoter, RNA polymerase II promoter, or RNA polymerase III promoter. In some embodiments, the donor nucleic acid molecule comprises a self-cleaving peptide such as a T2A or P2A site.

The donor nucleic acid molecule can be any size. In some embodiments, the heterologous nucleic acid molecule is about 10 bp-20 kb, about 100 bp-15 kb, or about 1 kb-10 kb. In some examples, the donor nucleic acid molecule is 10 bp, 25 bp, 50 bp, 100 bp, 200 bp, 500 bp, 800 bp, 1,000 bp, 1.5 kb, 2.0 kb, 3.0 kb, 5.0 kb, 7.5 kb, 10 kb, 12 kb, 15 kb, 20 kb or 30 kb in length.

In some embodiments, the heterologous nucleic acid molecule comprises a sequence having at least 50%, 60%, 70%, 80%, 85%, 90%, 95%, 97%, 98%, 99% or 100% identity to a target DNA sequence in the target genome, or a portion thereof.

As non-limiting examples, the heterologous gene or heterologous nucleic acid molecule comprises a polynucleotide sequence encoding a chimeric antigen receptor (CAR). The term “chimeric antigen receptor” or “CAR,” as used herein, refers to an artificial T cell surface receptor that is engineered to be expressed on an immune effector cell and specifically bind an antigen. CARs may be used as a therapy with adoptive cell transfer. Monocytes are removed from a patient (blood, tumor or ascites fluid) and modified so that they express receptors specific to a particular form of antigen. In some embodiments, the CARs have been expressed with specificity to a tumor associated antigen, for example. CARs may also comprise an intracellular activation domain, a transmembrane domain and an extracellular domain comprising a tumor associated antigen binding region. In some aspects, CARs comprise fusions of single-chain variable fragments (scFv) derived monoclonal antibodies, fused to CD3-zeta transmembrane and intracellular domain. The specificity of CAR designs may be derived from ligands of receptors (e.g., peptides). In some embodiments, a CAR can target cancers by redirecting a monocyte/macrophage expressing the CAR specific for tumor associated antigens.

In some embodiments, the co-stimulatory domain of the CAR can include, but is not limited to, a domain derived from CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3.

The CAR may comprise an antigen binding domain that binds to a tumor antigen, such as an antigen that is specific for a tumor or cancer of interest. In one embodiment, the tumor antigen of the present invention comprises one or more antigenic cancer epitopes. Nonlimiting examples of tumor associated antigens include CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8) aNeu5A (2-3)bDGalp(1-4)bDGlcp(1-1)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis (Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3) bDGalp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRLS); and immunoglobulin lambda-like polypeptide 1 (IGLL1).

A suitable transmembrane domain of particular use in an CAR described herein may be a transmembrane domain derived from CD28, 4-1BB/CD137, CD8 (e.g., CD8α), CD4, CD19, CD3 epsilon, CD45, CD5, CD9, CD16, CD22, CD33, CD37, CD64, CD80, CD86, CD134, CD137, CTLA4, PD-1, CD154, TCR alpha, TCR beta, gamma delta TCR or CD3 zeta and/or transmembrane regions containing functional variants thereof such as those retaining a substantial portion of the structural, e.g., transmembrane, properties thereof.

In some embodiments, the heterologous gene or heterologous nucleic acid molecule is an engineered T-cell receptor (TCR). In some embodiments, the heterologous nucleic acid molecule encodes a therapeutic protein. As used herein, the term “therapeutic protein” refers to any protein that, when administered to a subject directly or indirectly in the form of a translated nucleic acid, has a therapeutic, diagnostic, and/or prophylactic effect and/or elicits a desired biological and/or pharmacological effect.

In some embodiment, the heterologous nucleic acid is fused with a specific attB sequence or an attP sequence that is recognized by the large serine recombinase. In some examples, the heterologous nucleic acid comprises the first parapalindromic sequence and the second parapalindromic sequence of an attP sequence that a LSR binds to. The LSR then binds to the attP-attB complex formed between the attP sequence and the cognate attB sequence in the target genome and excise integration of the heterologous nucleic acid sequence into the target genome. In other examples, the heterologous nucleic acid comprises the first parapalindromic sequence and the second parapalindromic sequence of an attB sequence that a LSR binds to. The LSR then binds to the attP-attB complex formed between the attB sequence and the cognate attP sequence in the target genome and excise integration of the heterologous nucleic acid sequence into the target genome.

In some embodiments, the present system comprises a polynucleotide encoding a LSR or a variant thereof, a recognition sequence specific to the LSR and a heterologous (e.g., donor) nucleic acid sequence. In some embodiments, the system comprises an in vitro transcribed mRNA molecule encoding an LSR. In some embodiments, the system comprises an in vitro transcribed mRNA molecule encoding a heterologous polypeptide. In some embodiments, the system comprises circular mRNA. As used herein, the terms “circRNA” or “circular polyribonucleotide” or “circular RNA” are used interchangeably and refers to a polyribonucleotide that forms a circular structure through covalent bonds. In some embodiments, the heterologous nucleic acid sequence comprises a nanoplasmid. In some embodiments, the heterologous nucleic acid sequence comprises doggybone DNA or dbDNA™.

### Expression of the Large Serine Recombinase System

Recombinant expression of a large serine recombinase described herein, can include construction of an expression vector containing a polynucleotide that encodes the serine recombinase. Once a polynucleotide has been obtained, a vector for the production of the polypeptide can be produced by recombinant DNA technology using techniques known in the art. Known methods can be used to construct expression vectors containing polypeptide coding sequences and appropriate transcriptional and translational control signals. These methods include, for example, in vitro recombinant DNA techniques, synthetic techniques, and in vivo genetic recombination. In accordance with the present disclosure, there may be employed conventional molecular biology, microbiology, and recombinant DNA techniques within the skill of the art.

An expression vector can be transferred to a host cell by conventional techniques, and the transfected cells can then be cultured by conventional techniques to produce polypeptides.

In some embodiments, a nucleotide sequence encoding a large serine recombinase is operably linked to a control element, e.g., a transcriptional control element, such as a promoter. The transcriptional control element may be functional in either a eukaryotic cell, e.g., a mammalian cell; or a prokaryotic cell (e.g., bacterial or archaeal cell). In some embodiments, the eukaryotic cell is a human cell. In some embodiments, a nucleotide sequence encoding a novel large serine recombinase protein is operably linked to multiple control elements that allow expression of the encoded nucleotide sequence in both prokaryotic and eukaryotic cells.

A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular temperature, compound, or protein.), it may be a spatially restricted promoter (i.e., transcriptional control element, enhancer, etc.) (e.g., tissue specific promoter, cell type specific promoter, etc.), and it may be a temporally restricted promoter (i.e., the promoter is in the “ON” state or “OFF” state during specific stages of embryonic development or during specific stages of a biological process, e.g., hair follicle cycle in mice).

Suitable promoters can be derived from viruses and can therefore be referred to as viral promoters, or they can be derived from any organism, including prokaryotic or eukaryotic organisms. Suitable promoters can be used to drive expression by any RNA polymerase (e.g., pol I, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6) (Miyagishi et al., Nature Biotechnology 20, 497-500 (2002)), an enhanced U6 promoter (e.g., Xia et al., Nucleic Acids Res. 2003 Sep. 1; 31(17)), and/or a human HI promoter (HI).

Examples of inducible promoters include, but are not limited to T7 RNA polymerase promoter, T3 RNA polymerase promoter, Isopropyl-beta-D-thiogalactopyranoside (IPTG)-regulated promoter, lactose induced promoter, heat shock promoter, Tetracycline-regulated promoter (e.g., Tet-ON, Tet-OFF, etc.), Steroid-regulated promoter, Metal-regulated promoter, estrogen receptor-regulated promoter, etc. Inducible promoters can therefore be regulated by molecules including, but not limited to, doxycycline, RNA polymerase, e.g., T7 RNA polymerase, an estrogen receptor and/or an estrogen receptor fusion.

In some embodiments, the promoter is a spatially restricted promoter (i.e., cell type specific promoter, tissue specific promoter, etc.) such that in a multi-cellular organism, the promoter is active (i.e., “ON”) in a subset of specific cells. Spatially restricted promoters may also be referred to as enhancers, transcriptional control elements, control sequences, etc. Any convenient spatially restricted promoter may be used and the choice of suitable promoter (e.g., a brain specific promoter, a promoter that drives expression in a subset of neurons, a promoter that drives expression in the germline, a promoter that drives expression in the lungs, a promoter that drives expression in muscles, a promoter that drives expression in islet cells of the pancreas, etc.) will depend on the organism. Thus, a spatially restricted promoter can be used to regulate the expression of a nucleic acid encoding a subject site-directed polypeptide in a wide variety of different tissues and cell types, depending on the organism. Some spatially restricted promoters are also temporally restricted such that the promoter is in the “ON” state or “OFF” state during specific stages of embryonic development or during specific stages of a biological process (e.g., hair follicle cycle).

For illustration purposes, examples of spatially restricted promoters include, but are not limited to, neuron-specific promoters, adipocyte-specific promoters, cardiomyocyte-specific promoters, smooth muscle-specific promoters, photoreceptor-specific promoters, etc. Neuron-specific spatially restricted promoters include, but are not limited to, a neuron-specific enolase (NSE) promoter, an aromatic amino acid decarboxylase (AADC) promoter, a neurofilament promoter, a synapsin promoter, a thy-1 promoter, a serotonin receptor promoter, a tyrosine hydroxylase promoter (TH), a GnRH promoter, an L7 promoter, a DNMT promoter, an enkephalin promoter, a myelin basic protein (MBP) promoter, a Ca²⁺-calmodulin-dependent protein kinase II-alpha (CamKIIa) promoter and/or a CMV enhancer/platelet-derived growth factor-β promoter.

Adipocyte-specific spatially restricted promoters include, but are not limited to aP2 gene promoter/enhancer, e.g., a region from −5.4 kb to +21 bp of a human aP2 gene, a glucose transporter-4 (GLUT4) promoter, a fatty acid translocase (FAT/CD36) promoter, a stearoyl-CoA desaturase-1 (SCD1) promoter, a leptin promoter, and an adiponectin promoter, an adipsin promoter and/or a resistin promoter.

Cardiomyocyte-specific spatially restricted promoters include, but are not limited to control sequences derived from the following genes: myosin light chain-2, a-myosin heavy chain, AE3, cardiac troponin C, and/or cardiac actin.

Smooth muscle-specific spatially restricted promoters include, but are not limited to an SM22a promoter, a smoothelin promoter, and/or an a-smooth muscle actin promoter.

Photoreceptor-specific spatially restricted promoters include, but are not limited to, a rhodopsin promoter, a rhodopsin kinase promoter, a beta phosphodiesterase gene promoter, a retinitis pigmentosa gene promoter, an interphotoreceptor retinoid-binding protein (IRBP) gene enhancer, and/or an IRBP gene promoter.

In some embodiments, the expression vector is a viral vector, such as an adenoviral vector, an AAV vector, a lentiviral vector or a retroviral vector.

In some embodiments, the expression vector is non-viral vector.

In some embodiments, the system is construed as an in vitro transcribed messenger RNA for expression in a host cell or an organism.

In some embodiments, the polynucleotide encoding a large serine recombinase is constructed in an expressing vector, and the target nucleic acid molecule and the recognition sequence of the large serine recombinase are construed in a separate donor vector.

In some embodiments, the polynucleotide encoding a large serine recombinase, the target nucleic acid sequence and the recognition sequence are construed in a single vector.

### Large Serine Recombinase Mediated Recombination

The large serine recombinase system described herein can be used for genome modification. Large serine recombinase mediated recombination can lead to integration of a heterologous DNA (e.g., donor sequence) at a specific target locus resulting in a gene silencing event, replacement, an insertion of exogenous gene, or an alteration of the expression (e.g., an increase or a decrease) of a desired target gene. As used herein, the term “site specific modification” or “site specific recombination” refers to any changes to a genomic sequence around a target site in a genome.

Accordingly, in some embodiments, the large serine recombinase system described herein is used in a method of altering the expression of a target nucleic acid, e.g., disruption of expression of a target gene.

In some embodiments the large serine recombinase system described herein is used in a method of modifying a target nucleic acid in a desired target cell. In some embodiments, the invention provides methods for site-specific modification of a target nucleic acid in eukaryotic cells to effectuate a desired modification in gene expression.

In some embodiments, the large serine recombinase systems described herein can be used to modify a target nucleic acid (e.g., by inserting, deleting, or substituting one or more nucleic acid residues). For example, in some embodiments the systems described herein comprise an exogenous donor template nucleic acid (e.g., a DNA molecule or a RNA molecule), which comprises a desirable nucleic acid sequence. Upon resolution of a cleavage event induced with the system described herein, the molecular machinery of the cell will utilize the exogenous donor template nucleic acid in repairing and/or resolving the cleavage event. Alternatively, the molecular machinery of the cell can utilize an endogenous template in repairing and/or resolving the cleavage event. In some embodiments, the large serine recombinase systems described herein may be used to alter a target nucleic acid resulting in an insertion, a deletion, and/or a point mutation. In some embodiments, the insertion is a scarless insertion (i.e., the insertion of an intended nucleic acid sequence into a target nucleic acid resulting in no additional unintended nucleic acid sequence upon resolution of the cleavage event).

In some embodiments, after recombinase mediated recombination, the target site surrounding the integrated sequence contains a limited number of insertions or deletions, for example, in less than about 50% or 10% of integration events.

In some embodiments, the serine recombinase system of the present invention may result in a genomic modification (e.g., an insertion or deletion) at the target site (e.g., the site of insert DNA integration, e.g., adjacent to the integration of the insert DNA) comprising less than 20 nt, e.g., less than 20, 19, 18, 17, 16, 15, 14, 13, 12, 11, 10, 9, 8, 7, 6, 5, 4, 3, 2, or less than 1 nt of DNA. In some embodiments, a LSR system of this invention may result in an insertion at the target site (e.g., the site of insert DNA integration, e.g., adjacent to the integration of the insert DNA) comprising less than 20 nucleotides or base pairs, e.g., less than 20, 19, 18, 17, 16, 15, 14, 13, 12, 11, 10, 9, 8, 7, 6, 5, 4, 3, 2, or less than 1 nucleotides or base pairs of DNA. In some embodiments, the serine recombinase system of the present invention may result in a deletion at the target site (e.g., the site of insert DNA integration, e.g., adjacent to the integration of the insert DNA) comprising less than 20 nucleotides or base pairs, e.g., less than 20, 19, 18, 17, 16, 15, 14, 13, 12, 11, 10, 9, 8, 7, 6, 5, 4, 3, 2, or less than 1 nucleotide or base pair of genomic DNA. In some embodiments, the target site does not show multiple insertion events, e.g., head-to-tail or head-to-head duplications.

As discussed herein, the heterologous sequence is inserted into a target site in the genome of the cell. In some embodiments, the target site comprises, in order, (i) a first parapalindromic sequence), and (ii) a second parapalindromic sequence. Upon a LSR mediated recombination, a heterologous sequence is inserted to the target site between the first and the second parapalindromic sequence.

### Genome Target Sites

In some embodiments, the system of the present invention may be redirected to a defined target site in the human genome. In some embodiments, the target site can be any site in the target genome. In some embodiments, the system targets a genomic safe harbor target site, e.g., mediates an insertion of a heterogeneous nucleic acid sequence into a position that meets a safe harbor criteria. A genomic safe harbor site is a site in a host genome that is able to accommodate the integration of new genetic material, e.g., such that the inserted genetic element does not cause significant alterations of the host genome posing a risk to the host cell or organism.

Genomic safe harbor sites include, but are not limited to, any sites located more than 300 kb from a cancer-related gene; any sites located more than 300 kb from a miRNA/other functional small RNA; any sites located more than 50 kb from a 5′ gene end; any sites located more than 50 kb from a replication origin; any sites located more than 50 kb away from any ultraconserved element; any sites having low transcriptional activity (i.e. no mRNA +/−25 kb); any sites that are not in a copy number variable region; any sites in open chromatin; and any unique sites, with one copy in the human genome. Examples of genomic safe harbor sites in the human genome include the adeno-associated vims site 1, a naturally occurring site of integration of AAV vims on chromosome 19, the chemokine (C-C motif) receptor 5 (CCR5) gene, a chemokine receptor gene known as an HIV-1 co-receptor, the human ortholog of the mouse Rosa26 locus, the rDNA locus (e.g., 5S rDNA, 18S rDNA, 5.8S rDNA, and 28S rDNA loci), safe harbor sites described, e.g., in Pellenz et al., 2018.

In some embodiments the genomic safe harbor site is a naturally occurring safe harbor site. In some embodiments, a genomic sate harbor site is derived from the native target of a mobile genetic element, e.g., a recombinase, transposon, retrotransposon, or retrovirus. In some embodiments, a genomic safe harbor site is created using DNA modifying enzymes.

In some embodiments, a system of this invention may result in a genomic modification (e.g., an insertion or deletion) at the genome target site (e.g., the site where a heterogeneous nucleic acid sequence is integrated into the host genome by the LSR system,) comprising less than 20 nt, e.g., less than 20, 19, 18, 17, 16, 15, 14, 13, 12, 11, 10, 9, 8, 7, 6, 5, 4, 3, 2, or less than 1 nt flanking the insertion site of heterologous DNA.

In some embodiments, a target site shows less than 100 insert copies at the target site. In some embodiments, a target site shows more than two copies of the insert sequence are present in less than 95% of target sites containing inserts. In some embodiments, a target site shows multiple copies of the insert sequence. In some embodiments, the insertion of heterologous donor sequence results in formation of attL and attR sites, formed by the combination of portions of attB and attP sites.

### Pharmaceutical Compositions

In another aspect, provided by the present invention include compositions comprising a large serine recombinase or a variant thereof, and/or a large serine recombinase system as described herein. In some embodiments, a pharmaceutical composition comprising the same is provided. The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents (e.g., for specific delivery, increasing half-life, or other therapeutic compounds).

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.).

“Pharmaceutically acceptable vehicles” may be vehicles approved by a regulatory agency of the Federal or a state government or listed in the U.S. The term “vehicle” refers to a diluent, adjuvant, excipient, or carrier with which a compound of the invention is formulated for administration to a subject. Such pharmaceutical vehicles can be lipids, e.g. liposomes, e.g. liposome dendrimers; liquids, such as water and oils, including those of petroleum, animal, vegetable or synthetic origin, such as peanut oil, soybean oil, mineral oil, sesame oil and the like, saline; gum acacia, gelatin, starch paste, talc, keratin, colloidal silica, urea, and the like. In addition, auxiliary, stabilizing, thickening, lubricating and coloring agents may be used.

Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient,” “carrier,” “pharmaceutically acceptable carrier,” “vehicle,” or the like are used interchangeably herein.

Pharmaceutical compositions can comprise one or more pH buffering compounds to maintain the pH of the formulation at a predetermined level that reflects physiological pH, such as in the range of about 5.0 to about 8.0. The pH buffering compound used in the aqueous liquid formulation can be an amino acid or mixture of amino acids, such as histidine or a mixture of amino acids such as histidine and glycine. Alternatively, the pH buffering compound is preferably an agent which maintains the pH of the formulation at a predetermined level, such as in the range of about 5.0 to about 8.0, and which does not chelate calcium ions. Illustrative examples of such pH buffering compounds include, but are not limited to, imidazole and acetate ions. The pH buffering compound may be present in any amount suitable to maintain the pH of the formulation at a predetermined level.

Pharmaceutical compositions can also contain one or more osmotic modulating agents, i.e., a compound that modulates the osmotic properties (e.g, tonicity, osmolality, and/or osmotic pressure) of the formulation to a level that is acceptable to the blood stream and blood cells of recipient individuals. The osmotic modulating agent can be an agent that does not chelate calcium ions. The osmotic modulating agent can be any compound known or available to those skilled in the art that modulates the osmotic properties of the formulation. One skilled in the art may empirically determine the suitability of a given osmotic modulating agent for use in the inventive formulation. Illustrative examples of suitable types of osmotic modulating agents include, but are not limited to: salts, such as sodium chloride and sodium acetate; sugars, such as sucrose, dextrose, and mannitol; amino acids, such as glycine; and mixtures of one or more of these agents and/or types of agents. The osmotic modulating agent(s) may be present in any concentration sufficient to modulate the osmotic properties of the formulation.

Pharmaceutical compositions may be formulated into preparations in solid, semisolid, liquid or gaseous forms, such as tablets, capsules, powders, granules, ointments, solutions, suppositories, injections, inhalants, gels, microspheres, and aerosols.

In some embodiments, the pharmaceutical composition is formulated for delivery to a subject, e.g., for genome modification. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, and enhance solubility or uptake). Examples of such modifications or complexing agents include sulfate, gluconate, citrate and phosphate. The nucleic acids or polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Therapies that exhibit large therapeutic indices are preferred.

The data obtained from cell culture and/or animal studies can be used in formulating a range of dosages for humans. The dosage of the active ingredient typically lines within a range of circulating concentrations that include the ED50 with low toxicity. The dosage can vary within this range depending upon the dosage form employed and the route of administration utilized.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

In some embodiments, the pharmaceutical composition described herein is administered locally to a diseased site. In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In other embodiments, the pharmaceutical composition described herein is delivered in a controlled release system. In one embodiment, a pump can be used (See, e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et ah, 1989, J. Neurosurg. 71:105.) Other controlled release systems are discussed, for example, in Langer, supra.

In some embodiments, the pharmaceutical composition is formulated in accordance with routine procedures as a composition adapted for intravenous or subcutaneous administration to a subject, e.g., a human. In some embodiments, pharmaceutical composition for administration by injection are solutions in sterile isotonic use as solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration can be a liquid, e.g., sterile saline, lactated Ringer's or Hank's solution. In addition, the pharmaceutical composition can be in solid forms and re-dissolved or suspended immediately prior to use. Lyophilized forms are also contemplated. The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et ah, Gene Ther. 1999, 6:1438-47). Positively charged lipids such as N-[1-(2,3-dioleoyloxi) propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical composition described herein can be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising (a) a container containing a compound of the invention in lyophilized form and (b) a second container containing a pharmaceutically acceptable diluent (e.g., sterile used for reconstitution or dilution of the lyophilized compound of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers can be formed from a variety of materials such as glass or plastic. In some embodiments, the container holds a composition that is effective for treating a disease described herein and can have a sterile access port. For example, the container can be an intravenous solution bag or a vial having a stopper pierceable by a hypodermic injection needle. The active agent in the composition is a compound of the invention. In some embodiments, the label on or associated with the container indicates that the composition is used for treating the disease of choice. The article of manufacture can further comprise a second container comprising a pharmaceutically-acceptable buffer, such as phosphate-buffered saline, Ringer's solution, or dextrose solution. It can further include other materials desirable from a commercial and user standpoint, including other buffers, diluents, filters, needles, syringes, and package inserts with instructions for use.

In some embodiments, the large serine recombinase system is provided as part of a pharmaceutical composition. In some embodiments, the pharmaceutical composition comprises any of the fusion proteins provided herein. In some embodiments, the pharmaceutical composition comprises any of the complexes provided herein. In some embodiments pharmaceutical composition comprises a large serine recombinase, an attP or attB sequence, a heterologous DNA, a cationic lipid, and a pharmaceutically acceptable excipient. Pharmaceutical compositions can optionally comprise one or more additional therapeutically active substances.

### Engineered Cells

In some embodiments, the present invention provides engineered cells that are genetically modified using the systems and methods described herein. The engineered cells may be produced by introducing a serine large recombinase mediated DNA modification in the genome of the cell.

The engineered cells are any types of cells. In some embodiments, the cells are dividing cells. In some embodiments, the cells are non-dividing cells. In some embodiments, the cells are cell lines. In some embodiments, the cells are primary cells. In some embodiments, the cells are mammal cells including human cells. As non-limiting examples, the cells are immune cells (e.g., T cells, B cells, NK cells, macrophages etc), cancer cells, stem cells, progenitor cells, iPS cells and embryonic cells.

In some embodiments, an engineered cell comprises a heterologous sequence at one or more target sites.

Following the methods described above, a DNA region of interest may be cleaved and modified, i.e. “genetically modified”, ex vivo. In some embodiments, as when a selectable marker has been inserted into the DNA region of interest, the population of cells may be enriched for those comprising the genetic modification by separating the genetically modified cells from the remaining population. Prior to enriching, the “genetically modified” cells may make up only about 1% or more (e.g., 2% or more, 3% or more, 4% or more, 5% or more, 6% or more, 7% or more, 8% or more, 9% or more, 10% or more, 15% or more, or 20% or more) of the cellular population. Separation of “genetically modified” cells may be achieved by any convenient separation technique appropriate for the selectable marker used. For example, if a fluorescent marker has been inserted, cells may be separated by fluorescence activated cell sorting, whereas if a cell surface marker has been inserted, cells may be separated from the heterologous population by affinity separation techniques, e.g. magnetic separation, affinity chromatography, “panning” with an affinity reagent attached to a solid matrix, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g. propidium iodide). Any technique may be employed which is not unduly detrimental to the viability of the genetically modified cells. Cell compositions that are highly enriched for cells comprising modified DNA are achieved in this manner. By “highly enriched”, it is meant that the genetically modified cells will be 70% or more, 75% or more, 80% or more, 85% or more, 90% or more of the cell composition, for example, about 95% or more, or 98% or more of the cell composition. In other words, the composition may be a substantially pure composition of genetically modified cells.

Genetically modified cells produced by the methods described herein may be used immediately. Alternatively, the cells may be frozen at liquid nitrogen temperatures and stored for long periods of time, being thawed and capable of being reused. In such cases, the cells will usually be frozen in 10% dimethylsulfoxide (DMSO), 50% serum, 40% buffered medium, or some other such solution as is commonly used in the art to preserve cells at such freezing temperatures, and thawed in a manner as commonly known in the art for thawing frozen cultured cells.

The genetically modified cells may be cultured in vitro under various culture conditions. The cells may be expanded in culture, i.e. grown under conditions that promote their proliferation. Culture medium may be liquid or semi-solid, e.g. containing agar, methylcellulose, etc. The cell population may be suspended in an appropriate nutrient medium, such as Iscove's modified DMEM or RPMI 1640, normally supplemented with fetal calf serum (about 5-10%), L-glutamine, a thiol, particularly 2-mercaptoethanol, and antibiotics, e.g. penicillin and streptomycin. The culture may contain growth factors to which the regulatory T cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors.

Exemplary engineered cells include CAR T cells, CAR NK cells and other engineered immune cells for immunotherapy. In some aspects, the CAR-T cells are autologous T cells. In some aspects, the CAR T cells are allogeneic.

Cells that have been genetically modified in this way may be transplanted to a subject for purposes such as gene therapy, e.g., to treat a disease or as an antiviral, antipathogenic, or anticancer therapeutic, for the production of genetically modified organisms in agriculture, or for biological research. The subject may be a neonate, a juvenile, or an adult. Of particular interest are mammalian subjects. Mammalian species that may be treated with the present methods include canines and felines; equines; bovines; ovines; etc. and primates, particularly humans. Animal models, particularly small mammals (e.g., mouse, rat, guinea pig, hamster, lagomorpha (e.g., rabbit), etc.) may be used for experimental investigations.

Cells may be provided to the subject alone or with a suitable substrate or matrix, e.g. to support their growth and/or organization in the tissue to which they are being transplanted. Usually, at least 1×10³ cells will be administered, for example 5×10³ cells, 1×10⁴ cells, 5×10⁴ cells, 1×10⁵ cells, 1×10⁶ cells or more. The cells may be introduced to the subject via any of the following routes: parenteral, subcutaneous, intravenous, intracranial, intraspinal, intraocular, or into spinal fluid. The cells may be introduced by injection, catheter, or the like. Cells may also be introduced into an embryo (e.g., a blastocyst) for the purpose of generating a transgenic animal (e.g., a transgenic mouse).

The number of administrations of treatment to a subject may vary. Introducing the genetically modified cells into the subject may be a one-time event; but in certain situations, such treatment may elicit improvement for a limited period of time and require an on-going series of repeated treatments. In other situations, multiple administrations of the genetically modified cells may be required before an effect is observed. The exact protocols depend upon the disease or condition, the stage of the disease and parameters of the individual subject being treated.

### Delivery Systems

The large serine recombinase systems described herein, or components thereof, nucleic acid molecules thereof, and/or nucleic acid molecules encoding or providing components thereof, can be delivered by various delivery systems such as vectors, e.g., plasmids and delivery vectors. Exemplary embodiments are described below. The large serine recombinase systems can be encoded on a nucleic acid that is contained in a viral vector. Viral vectors can include lentivirus, Adenovirus, Retrovirus, and Adeno-associated viruses (AAVs). Viral vectors can be selected based on the application. For example, AAVs are commonly used for gene delivery in vivo due to their mild immunogenicity. Adenoviruses are commonly used as vaccines because of the strong immunogenic response they induce. Packaging capacity of the viral vectors can limit the size of the large serine recombinase that can be packaged into the vector. For example, the packaging capacity of the AAVs is ˜4.5 kb including two 145 base inverted terminal repeats (ITRs).

AAV is a small, single-stranded DNA dependent virus belonging to the parvovirus family. The 4.7 kb wild-type (wt) AAV genome is made up of two genes that encode four replication proteins and three capsid proteins, respectively, and is flanked on either side by 145-bp inverted terminal repeats (ITRs). The virion is composed of three capsid proteins, Vp1, Vp2, and Vp3, produced in a 1:1:10 ratio from the same open reading frame but from differential splicing (Vp1) and alternative translational start sites (Vp2 and Vp3, respectively). Vp3 is the most abundant subunit in the virion and participates in receptor recognition at the cell surface defining the tropism of the virus. A phospholipase domain, which functions in viral infectivity, has been identified in the unique N terminus of Vp1.

Similar to wt AAV, recombinant AAV (rAAV) utilizes the cis-acting 145-bp ITRs to flank vector transgene cassettes, providing up to 4.5 kb for packaging of foreign DNA. Subsequent to infection, rAAV can express a fusion protein of the invention and persist without integration into the host genome by existing episomally in circular head-to-tail concatemers. Although there are numerous examples of rAAV success using this system, in vitro and in vivo, the limited packaging capacity has limited the use of AAV-mediated gene delivery when the length of the coding sequence of the gene is equal or greater in size than the wt AAV genome.

The small packaging capacity of AAV vectors makes the delivery of a number of genes that exceed this size and/or the use of large physiological regulatory elements challenging. These challenges can be addressed, for example, by dividing the protein(s) to be delivered into two or more fragments, wherein the N-terminal fragment is fused to a split intein-N and the C-terminal fragment is fused to a split intein-C. These fragments are then packaged into two or more AAV vectors. As used herein, “intein” refers to a self-splicing protein intron (e.g., peptide) that ligates flanking N-terminal and C-terminal exteins (e.g., fragments to be joined). The use of certain inteins for joining heterologous protein fragments is described, for example, in Wood et al., J. Biol. Chem. 289(21); 14512-9 (2014). For example, when fused to separate protein fragments, the inteins IntN and IntC recognize each other, splice themselves out and simultaneously ligate the flanking N- and C-terminal exteins of the protein fragments to which they were fused, thereby reconstituting a full-length protein from the two protein fragments. Other suitable inteins will be apparent to a person of skill in the art.

In some embodiments, the serine recombinase system of the invention can vary in length. In some embodiments, a protein fragment ranges from 500 amino acids to about 5000 amino acids in length. In some embodiments, a protein fragment ranges from about 500 amino acids to about 4000 amino acids in length. In some embodiments, a protein fragment ranges from about 500 amino acids to about 3000 amino acids in length. In some embodiments, a protein fragment ranges from about 500 amino acids to about 2000 amino acids in length. In some embodiments, a protein fragment ranges from about 500 amino acids to about 1000 amino acids in length. Suitable protein fragments of other lengths will be apparent to a person of skill in the art.

In some embodiments, a portion or fragment of a fusion protein is fused to an intein and fused to an AAV capsid protein. The intein, nuclease and capsid protein can be fused together in any arrangement (e.g., nuclease-intein-capsid, intein-nuclease-capsid, capsid-intein-nuclease, etc.). In some embodiments, the N-terminus of an intein is fused to the C-terminus of a fusion protein and the C-terminus of the intein is fused to the N-terminus of an AAV capsid protein.

In one embodiment, dual AAV vectors are generated by splitting a large transgene expression cassette in two separate halves (5′ and 3′ ends, or head and tail), where each half of the cassette is packaged in a single AAV vector (of <5 kb). The re-assembly of the full-length transgene expression cassette is then achieved upon co-infection of the same cell by both dual AAV vectors followed by: (1) homologous recombination (HR) between 5′ and 3′ genomes (dual AAV overlapping vectors); (2) ITR-mediated tail-to-head concatemerization of 5′ and 3′ genomes (dual AAV trans-splicing vectors); or (3) a combination of these two mechanisms (dual AAV hybrid vectors). The use of dual AAV vectors in vivo results in the expression of full-length proteins. The use of the dual AAV vector platform represents an efficient and viable gene transfer strategy for transgenes of >4.7 kb in size.

The disclosed strategies for designing large serine recombinase systems described herein can be useful for generating systems capable of being packaged into a viral vector. The use of RNA or DNA viral based systems for the delivery of a recombinase takes advantage of highly evolved processes for targeting a virus to specific cells in culture or in the host and trafficking the viral payload to the nucleus or host cell genome. Viral vectors can be administered directly to cells in culture, patients (in vivo), or they can be used to treat cells in vitro, and the modified cells can optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (See, e.g., Buchscher et al., J. Virol. 66:2731-2739 (1992); Johann et al., J. Virol. 66:1635-1640 (1992); Sommerfelt et al., Virol. 176:58-59 (1990); Wilson et al., J. Virol. 63:2374-2378 (1989); Miller et al., J. Virol. 65:2220-2224 (1991); PCT/US94/05700).

Retroviral vectors, especially lentiviral vectors, can require polynucleotide sequences smaller than a given length for efficient integration into a target cell. For example, retroviral vectors of length greater than 9 kb can result in low viral titers compared with those of smaller size. In some aspects, a system of the present disclosure is of sufficient size so as to enable efficient packaging and delivery into a target cell via a retroviral vector. In some cases, a large serine recombinase is of a size so as to allow efficient packing and delivery even when expressed together with heterologous DNA.

In applications where transient expression is preferred, adenoviral based systems can be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors can also be used to transduce cells with target nucleic acids, e.g., in the in vitro production of nucleic acids and peptides, and for in vivo and ex vivo gene therapy procedures (See, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). The construction of recombinant AAV vectors is described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

A large serine recombinase system described herein can therefore be delivered with viral vectors. One or more components of the large serine recombinase system can be encoded on one or more viral vectors. For example, a large serine recombinase and donor sequence can be encoded on a single viral vector. In other cases, the large serine recombinase and donor sequence are encoded on different viral vectors.

The combination of components encoded on a viral vector can be determined by the cargo size constraints of the chosen viral vector.

### Non-Viral Delivery

Non-viral delivery approaches for large serine recombinases are also available. One important category of non-viral nucleic acid vectors are nanoparticles, which can be organic or inorganic. Nanoparticles are well known in the art. Any suitable nanoparticle design can be used to deliver genome editing system components or nucleic acids encoding such components. For instance, organic (e.g. lipid and/or polymer) nanoparticles can be suitable for use as delivery vehicles in certain embodiments of this disclosure. Exemplary lipids for use in nanoparticle formulations, and/or gene transfer are shown in Table 1 (below).

TABLE 1
Lipids Used for Gene Transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3-phosphatidylethanolamine | DOPE | Helper |
| Cholesterol | | Helper |
| N-[1-(2,3-Dioleyloxy)prophyl]N,N,N-trimethylammonium | DOTMA | Cationic |
| chloride | | |
| 1,2-Dioleoyloxy-3-trimethylammonium-propane | DOTAP | Cationic |
| Dioctadecylamidoglycylspermine | DOGS | Cationic |
| N-(3-Aminopropyl)-N,N-dimethyl-2,3-bis(dodecyloxy)-1- | GAP-DLRIE | Cationic |
| propanaminium bromide | | |
| Cetyltrimethylammonium bromide | CTAB | Cationic |
| 6-Lauroxyhexyl ornithinate | LHON | Cationic |
| 1-(2,3-Dioleoyloxypropyl)-2,4,6-trimethylpyridinium | 2Oc | Cationic |
| 2,3-Dioleyloxy-N-[2(sperminecarboxamido-ethyl]-N,N- | DOSPA | Cationic |
| dimethyl-1-propanaminium trifluoroacetate | | |
| 1,2-Dioley1-3-trimethylammonium-propane | DOPA | Cationic |
| N-(2-Hydroxyethyl)-N,N-dimethyl-2,3-bis(tetradecyloxy)-1- | MDRIE | Cationic |
| propanaminium bromide | | |
| Dimyristooxypropyl dimethyl hydroxyethyl ammonium bromide | DMRI | Cationic |
| 3β-[N-(N′,N′-Dimethylaminoethane)-carbamoyl]cholesterol | DC-Chol | Cationic |
| Bis-guanidium-tren-cholesterol | BGTC | Cationic |
| 1,3-Diodeoxy-2-(6-carboxy-spermyl)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2-hydroxyethyl)]- | CLIP-1 | Cationic |
| dimethylammonium chloride | | |
| rac-[2(2,3-Dihexadecyloxypropyl- | CLIP-6 | Cationic |
| oxymethyloxy)ethyl]trimethylammoniun bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylpho sphocholine | DSEPC | Cationic |
| N-Palmitoyl D-erythro-sphingosyl carbamoyl-spermine | CCS | Cationic |
| N-t-Butyl-N0-tetradecyl-3-tetradecylaminopropionamidine | diC14-amidine | Cationic |
| Octadecenolyoxy[ethyl-2-heptadecenyl-3 hydroxyethyl] | DOTIM | Cationic |
| imidazolinium chloride | | |
| N1-Cholesteryloxycarbonyl-3,7-diazanonane-1,9-diamine | CDAN | Cationic |
| 2-(3-[Bis(3-amino-propyl)-amino]propylamino)-N- | RPR209120 | Cationic |
| ditetradecylcarbamoylme-ethyl-acetamide | | |
| 1,2-dilinoleyloxy-3-dimethylaminopropane | DLinDMA | Cationic |
| 2,2-dilinoley1-4-dimethylaminoethyl-[1,3]-dioxolane | DLin-KC2- | Cationic |
| | DMA | |
| dilinoleyl-methyl-4-dimethylaminobutyrate | DLin-MC3- | Cationic |
| | DMA | |

Table 1 lists exemplary polymers for use in gene transfer and/or nanoparticle formulations.

TABLE 1
Polymers Used for Gene Transfer

| Polymer | Abbreviation |
|---|---|
| Poly(ethylene)glycol | PEG |
| Polyethylenimine | PEI |
| Dithiobis (succinimidylpropionate) | DSP |
| Dimethyl-3,3′-dithiobispropionimidate | DTBP |
| Poly(ethylene imine)biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amidoethylenimine) | SS-PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-Dodacylated chitosan | |
| Histone | |
| Collagen | |
| Dextran-spermine | D-SPM |

Table 2 summarizes delivery methods for a polynucleotide encoding a large serine recombinase described herein.

TABLE 2

| | | Delivery into | | | Type of |
|---|---|---|---|---|---|
| | | Non-Dividing | Duration of | Genome | Molecule |
| Delivery | Vector/Mode | Cells | Expression | Integration | Delivered |
| Physical | (e.g., | YES | Transient | NO | Nucleic Acids |
| | electroporation, | | | | and Proteins |
| | particle gun, | | | | |
| | Calcium | | | | |
| | Phosphate | | | | |
| | transfection | | | | |
| Viral | Retrovirus | NO | Stable | YES | RNA |
| | Lentivirus | YES | Stable | YES/NO with | RNA |
| | | | | modification | |
| | Adenovirus | YES | Transient | NO | DNA |
| | Adeno- | YES | Stable | NO | DNA |
| | Associated | | | | |
| | Virus (AAV) | | | | |
| | Vaccinia Virus | YES | Very | NO | DNA |
| | | | Transient | | |
| | Herpes Simplex | YES | Stable | NO | DNA |
| | Virus | | | | |
| Non-Viral | Cationic | YES | Transient | Depends on | Nucleic Acids |
| | Liposomes | | | what is | and Proteins |
| | | | | delivered | |
| | Polymeric | YES | Transient | Depends on | Nucleic Acids |
| | Nanoparticles | | | what is | and Proteins |
| | | | | delivered | |
| Biological | Attenuated | YES | Transient | NO | Nucleic Acids |
| Non-Viral | Bacteria | | | | |
| Delivery | Engineered | YES | Transient | NO | Nucleic Acids |
| Vehicles | Bacteriophages | | | | |
| | Mammalian | YES | Transient | NO | Nucleic Acids |
| | Virus-like | | | | |
| | Particles | | | | |
| | Biological | YES | Transient | NO | Nucleic Acids |
| | liposomes: | | | | |
| | Erythrocyte | | | | |
| | Ghosts and | | | | |
| | Exosomes | | | | |

In some embodiments, the LSR system, or polynucleotides comprising a LSR system contemplated in the present disclosure, is encapsulated in a lipid nanoparticle for in vitro, ex vivo and/or in vivo delivery. In some examples, the LSR system or the polynucleotide comprising the LSR system is delivered into a cell by electroporation.

In some embodiments, the LSR system may be co-delivered into a cell, a tissue or a subject with a heterogeneous nucleic acid, e.g., a polynucleotide encoding a chimeric antigen receptor (CAR); the LSR system and the polynucleotide encoding the CAR are encapsulated into a single LNP, or into different LNPs separately.

In some embodiments, the LSR system comprises a circular nucleic acid molecule (e.g., circRNA and circDNA). In some embodiments, the circular nucleic acid molecule may be encapsulated in a LNP for delivery.

A promoter used to drive the system can include AAV ITR. This can be advantageous for eliminating the need for an additional promoter element, which can take up space in the vector. The additional space freed up can be used to drive the expression of additional elements, such as a guide nucleic acid or a selectable marker. ITR activity is relatively weak, so it can be used to reduce potential toxicity due to over expression of the chosen nuclease.

Any suitable promoter can be used to drive expression of the large serine recombinase. For ubiquitous expression, promoters that can be used include CMV, CAG, CBh, PGK, SV40, Ferritin heavy or light chains, etc. For brain or other CNS cell expression, suitable promoters can include: SynapsinI for all neurons, CaMKIIalpha for excitatory neurons, GAD67 or GAD65 or VGAT for GABAergic neurons, etc. For liver cell expression, suitable promoters include the Albumin promoter. For lung cell expression, suitable promoters can include SP-B. For endothelial cells, suitable promoters can include ICAM. For hematopoietic cells suitable promoters can include IFNbeta or CD45. For Osteoblasts suitable promoters can include OG-2.

In some cases, a large serine recombinase of the present disclosure is of small enough size to allow separate promoters to drive expression of the large serine recombinase and a compatible recognition sequence acid within the same nucleic acid molecule. For instance, a vector or viral vector can comprise a first promoter operably linked to a nucleic acid encoding the large serine recombinase and a second promoter operably linked to the heterologous nucleic acid.

The promoter used to drive expression of a guide nucleic acid can include: Pol III promoters such as U6 or H1 Use of Pol II promoter and intronic cassettes to express gRNA Adeno Associated Virus (AAV).

A large serine recombinase described herein with or without one or more guide nucleic can be delivered using adeno associated virus (AAV), lentivirus, adenovirus or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For example, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus. For plasmid delivery, the route of administration, formulation and dose can be as in U.S. Pat. No. 5,846,946 and as in clinical studies involving plasmids. Doses can be based on or extrapolated to an average 70 kg individual (e.g., a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into the tissue of interest. For cell-type specific editing, the expression of the serine recombinase and optional donor nucleic acid can be driven by a cell-type specific promoter.

For in vivo delivery, AAV can be advantageous over other viral vectors. In some cases, AAV allows low toxicity, which can be due to the purification method not requiring ultra-centrifugation of cell particles that can activate the immune response. In some cases, AAV allows low probability of causing insertional mutagenesis because it doesn't integrate into the host genome.

AAV has a packaging limit of 4.5 or 4.75 Kb. Constructs larger than 4.5 or 4.75 Kb can lead to significantly reduced virus production.

An AAV can be AAV1, AAV2, AAV5 or any combination thereof. One can select the type of AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. A tabulation of certain AAV serotypes as to these cells can be found in Grimm, D. et al, J. Virol. 82:5887-5911 (2008)).

Lentiviruses are complex retroviruses that have the ability to infect and express their genes in both mitotic and post-mitotic cells. The most commonly known lentivirus is the human immunodeficiency virus (HIV), which uses the envelope glycoproteins of other viruses to target a broad range of cell types.

Lentiviruses can be prepared as follows. After cloning pCasES10 (which contains a lentiviral transfer plasmid backbone), HEK293FT at low passage (p=5) were seeded in a T-75 flask to 50% confluence the day before transfection in DMEM with 10% fetal bovine serum and without antibiotics. After 20 hours, media is changed to OptiMEM (serum-free) media and transfection was done 4 hours later. Cells are transfected with 10 μg of lentiviral transfer plasmid (pCasES10) and the following packaging plasmids: 5 μg of pMD2.G (VSV-g pseudotype), and 7.5 μg of psPAX2 (gag/pol/rev/tat). Transfection can be done in 4 mL OptiMEM with a cationic lipid delivery agent (50 μl Lipofectamine 2000 and 100 ul Plus reagent). After 6 hours, the media is changed to antibiotic-free DMEM with 10% fetal bovine serum. These methods use serum during cell culture, but serum-free methods are preferred.

Lentivirus can be purified as follows. Viral supernatants are harvested after 48 hours. Supernatants are first cleared of debris and filtered through a 0.45 μm low protein binding (PVDF) filter. They are then spun in an ultracentrifuge for 2 hours at 24,000 rpm. Viral pellets are resuspended in 50 μl of DMEM overnight at 4° C. They are then aliquoted and immediately frozen at −80° C.

In another embodiment, minimal non-primate lentiviral vectors based on the equine infectious anemia virus (EIAV) are also contemplated. In another embodiment, RetinoStat®, an equine infectious anemia virus-based lentiviral gene therapy vector that expresses angiostatic proteins endostatin and angiostatin that is contemplated to be delivered via a subretinal injection. In another embodiment, use of self-inactivating lentiviral vectors is contemplated.

To enhance expression and reduce possible toxicity, the system can be modified to include one or more modified nucleoside e.g., using pseudo-U or 5-Methyl-C.

The disclosure in some embodiments comprehends a method of modifying a cell or organism. The cell can be a prokaryotic cell or a eukaryotic cell. The cell can be a mammalian cell. The mammalian cell many be a non-human primate, bovine, porcine, rodent or mouse cell. The modification introduced to the cell by the recombinase, compositions and methods of the present disclosure can be such that the cell and progeny of the cell are altered for improved production of biologic products such as an antibody, starch, alcohol or other desired cellular output. The modification introduced to the cell by the methods of the present disclosure can be such that the cell and progeny of the cell include an alteration that changes the biologic product produced.

The system can comprise one or more different vectors. In an aspect, the large serine recombinase and/or heterologous DNA is codon optimized for expression the desired cell type, preferentially a eukaryotic cell, preferably a mammalian cell or a human cell.

In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/(visited Jul. 9, 2002), and these tables can be adapted in a number of ways. See, Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g., 1, 2, 3, 4, 5, 10, 15, 20, 25, 50, or more, or all codons) in a sequence encoding an engineered nuclease correspond to the most frequently used codon for a particular amino acid.

Packaging cells are typically used to form virus particles that are capable of infecting a host cell. Such cells include 293 cells, which package adenovirus, and psi.2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA can be packaged in a cell line, which contains a helper plasmid encoding the other AAV genes, namely rep and cap, but lacking ITR sequences. The cell line can also be infected with adenovirus as a helper. The helper virus can promote replication of the AAV vector and expression of AAV genes from the helper plasmid. The helper plasmid in some cases is not packaged in significant amounts due to a lack of ITR sequences. Contamination with adenovirus can be reduced by, e.g., heat treatment to which adenovirus is more sensitive than AAV.

### Applications and Methods of Use

Using the systems described herein, optionally using any of compositions and delivery modalities described herein (including nanoparticle delivery modalities, such as lipid nanoparticles, and viral delivery modalities, such as AAVs), the invention also provides applications for modifying a DNA molecule in the genome of a cell, whether in vitro, ex vivo, in situ, or in vivo, e.g.,, in a tissue in an organism, such as a subject including mammalian subjects, such as a human. In accordance, one aspect of the present invention provides a method for modifying a DNA sequence in a target genome; the method comprising introducing into the target genome a serine recombinase as described herein or a variant thereof, or a system comprising a serine recombinase.

In some embodiments, the target genome is a human genome.

In some embodiments, the method or system is used to control the expression of a target coding mRNA (i.e., a protein encoding gene) where binding results in increased or decreased gene expression. In some embodiments, the method or system is used to control gene regulation by integrating heterologous DNA into genetic regulatory elements such as promoters or enhancers, or integrating heterologous promoters at other target locations.

In accordance, a heterogeneous sequence to be inserted into a host genome is also provided. In some embodiments, the heterogeneous sequence and the LSR system are delivered into the host genome simultaneously. In other embodiments, the heterogeneous sequence and the LSR system are delivered into the host genome separately. In some embodiments, the heterogeneous sequence is inserted at the cleavage site induced by the LSR.

As non-limiting examples, the method or system is used to generate CAR expressing cells; the method and/or system can be used to control the expression of a CAR targeting a tumor specific antigen.

The heterogeneous sequence may be provided to the cell as single-stranded DNA, single-stranded RNA, double-stranded DNA, double-stranded RNA, circular RNA circular DNA, nanoplasmid, minicircle DNA or doggybone DNA (dbDNA™). It may be introduced into a cell in linear or circular form. If introduced in linear form, the ends of the donor sequence may be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. Additional methods for protecting exogenous polynucleotides from degradation include, but are not limited to, addition of terminal amino group(s) and the use of modified internucleotide linkages such as, for example, phosphorothioates, phosphor amidates, and O-methyl ribose or deoxyribose residues. As an alternative to protecting the termini of a linear donor sequence, additional lengths of sequence may be included outside of the regions of homology that can be degraded without impacting recombination. A donor sequence can be introduced into a cell as part of a vector molecule having additional sequences such as, for example, replication origins, promoters and genes encoding antibiotic resistance. Moreover, donor sequences can be introduced as naked nucleic acid, as nucleic acid complexed with an agent such as a liposome or poloxamer, or can be delivered by viruses (e.g., adenovirus, AAV), as described above for nucleic acids encoding a DNA-targeting RNA and/or site-directed modifying polypeptide and/or donor polynucleotide.

In some embodiments, the method or system is used to control the expression of a target non-coding RNA, including tRNA, rRNA, snoRNA, siRNA, miRNA, and long ncRNA.

In some embodiments, the method or system is used for site-specific editing of a target DNA, e.g., insertion of template DNA into a target DNA. In some embodiments, the system is used for of generating an edit, e.g., an insertion, that is present at the target site with a higher frequency than any other site in the genome, e.g., an insertion in a target site at a frequency of at least 2, 3, 4, 5, 10, 50, 100, or 1000-fold that of the frequency at all other sites in the genome.

In some embodiments, the large serine recombinase method or system is used for correction of pathogenic mutations by insertion of beneficial clinical variants or suppressor mutations.

In some embodiments, the system is able to modify a target genome without introducing undesirable mutations.

In some embodiments, efficiency of integration events can be used as a measure of editing of target sites by a LSR system of the present invention. In some examples, the LSR system described herein can integrate a heterologous sequence in a fraction of target sites. The LSR system is capable of editing at least 1%, 2%, 5%, 10%, 20%, 30%, 40%, 50%, 60%, 70%, 80%, 90%, 95%, 96%, 97%, 98%, 99%, 99.5%, 99.9% or 100% of target loci as measured by the present assay (e.g., NGS).

In some embodiments, a LSR system is capable of editing cells at an average copy number of at least 0.1, e.g., at least 0.1, 0.5, 1, 2, 3, 4, 5, 10, or 100 copies per genome as normalized to a reference gene.

In some embodiments, a ratio of on-target integration and off-target integration is measured for determining the efficacy of a LSR system.

### Therapeutic Applications

The large serine recombinase methods or systems described herein can have various therapeutic applications. Accordingly, in some embodiments, a method of treating a disorder or a disease in a subject in need thereof is provided; the method comprising administering to the subject a large serine recombinase system for modifying a DNA sequence template in the subject in need. Exemplary therapeutic modifications include integrating therapeutic nucleic acid molecules into a DNA sequence template, providing expression of a therapeutic transgene in individuals with loss-of-function mutations, replacing gain-of-function mutations with normal transgenes, providing regulatory sequences to eliminate gain-of-function mutation expression, and/or controlling the expression of operably linked genes, transgenes and systems thereof.

In some embodiments, the heterologous sequence is a therapeutic agent, e.g., a therapeutic transgene expressing a therapeutic agent/protein.

Exemplary therapeutic proteins include replacement blood factors (e.g., Factor II, V, VII, X, XI, XII or XIII) and replacement enzymes, e.g., lysosomal enzymes. In some examples, the compositions, LSR systems and methods described herein are useful to express, in a target human genome, agalsidase alpha or beta for treatment of Fabry Disease; imiglucerase, taliglucerase alfa, velaglucerase alfa, or alglucerase for Gaucher Disease; sebelipase alpha for lysosomal acid lipase deficiency (Wolman disease/CESD); laronidase, idursulfase, elosulfase alpha, or galsulfase for mucopolysaccharidoses; alglucosidase alpha for Pompe disease, factor I, II, V, VII, X, XI, XII or XIII for blood factor deficiencies.

In some embodiments, the compositions, LSR systems and methods described herein can be used to modify the genome in the subject to express a heterologous sequence encoding an intracellular protein (e.g., a cytoplasmic protein, a nuclear protein, an organellar protein such as a mitochondrial protein or lysosomal protein, or a membrane protein). In some examples, the heterologous sequence encode a membrane protein, e.g., a membrane protein other than a CAR, and/or an endogenous human membrane protein, an extracellular protein, an enzyme, a structural protein, a signaling protein, a regulatory protein, a transport protein, a sensory protein, a motor protein, a defense protein, or a storage protein.

In some embodiments, the compositions, LSR systems and methods described herein can be used to modify the genome in the subject to express a heterologous sequence encoding a chimeric antigen receptor (CAR), a T cell receptor, a B cell receptor, or an antibody.

In some embodiments, the compositions, LSR systems and methods described herein are used for immunotherapy, for example by modifying an immune cell to express a CAR or a TCR against a cancer specific antigen. The immune cells may be T cells, including any subpopulation of T-cells, e.g., CD4+, CD8+, gamma-delta, naive T cells, stem cell memory T cells, central memory T cells, or a mixture of subpopulations. In some embodiments, the immune cells are NK cells. In other examples, the compositions, LSR systems and methods described herein can be used to deliver a CAR or TCR to natural killer T (NKT) cells, and progenitor cells, e.g., progenitor cells of T, NK, or NKT cells.

In some embodiments, the immune cells comprise a CAR specific to a tumor or a pathogen antigen selected from a group consisting of AChR (fetal acetylcholine receptor), ADGRE2, AFP (alpha fetoprotein), BAFF-R, BCMA, CAIX (carbonic anhydrase IX), CCR1, CCR4, CEA (carcinoembryonic antigen), CD3, CD5, CD8, CD7, CD10, CD13, CD14, CD15, CD19, CD20, CD22, CD30, CD33, CFFI, CD34, CD38, CD41, CD44, CD49f, CD56, CD61, CD64, CD68, CD70,CD74, CD99,CD117, CD123, CD133, CD138, CD44v6, CD267, CD269, CDS, CFEC12A, CS1, EGP-2 (epithelial glycoprotein-2), EGP-40 (epithelial glycoprotein-40), EGFR (HERI), EGFR-VIII, EpCAM (epithelial cell adhesion molecule), EphA2, ERBB2 (HER2, human epidermal growth factor receptor 2), ERBB3, ERBB4, FBP (folate-binding protein), Flt3 receptor, folate receptor-a, GD2 (ganglioside G2), GD3 (ganglioside G3), GPC3 (glypican-3), GPI00, hTERT (human telomerase reverse transcriptase), ICAM-1, integrin B7, interleukin 6 receptor, IF13Ra2 (interleukin-13 receptor 30 subunit alpha-2), kappa-light chain, KDR (kinase insert domain receptor), FeY (Fewis Y), FICAM (FI cell adhesion molecule), FIFRB2 (leukocyte immunoglobulin like receptor B2), MARTI, MAGE-A1 (melanoma associated antigen Al), MAGE-A3, MSLN (mesothelin), MUC16 (mucin 16), MUCI (mucin I), KG2D ligands, NY-ESO-1 (cancer-testis antigen), PRI (proteinase 3), TRBCI, TRBC2, TFM-3, TACI, tyrosinase, survivin, hTERT, oncofetal antigen (h5T4), p53, PSCA (prostate stem cell antigen), PSMA (pro state-specific membrane antigen), hRORI, TAG-72 (tumor-associated glycoprotein 72), VEGF-R2 (vascular endothelial growth factor R2), WT-1 (Wilms tumor protein), and antigens of HIV (human immunodeficiency virus), hepatitis B, hepatitis C, CMV (cytomegalovirus), EBV (Epstein-Barr virus), HPV (human papilloma virus).

In some embodiments, immune cells, e.g., T-cells, NK cells, NKT cells, or progenitor cells are modified ex vivo and then delivered to a patient. In some embodiments, a LSR system is delivered by one of the methods mentioned herein, and immune cells, e.g., T-cells, NK cells, NKT cells, or progenitor cells are modified in vivo in the patient.

In one aspect, the methods or systems described herein can be used for treating a disease caused by overexpression of a disease gene, mutations in a disease gene and altered function of a disease gene.

The methods or systems described herein can also be used to treat a cancer in a subject (e.g., a human subject). For example, the large serine recombinases can integrate a lethal gene or a conditional lethal gene in cancer cells to induce cell death in the cancer cells (e.g., via apoptosis).

In some embodiments, a LSR system of the present invention can be used to make multiple modifications to a target cell, either simultaneously or sequentially. In some embodiments, a LSR system of the present invention can be used to further modify an already modified cell.

In some embodiments, a LSR system of the present invention can be used to modify a cell edited by a complementary technology, e.g., a gene edited cell, e.g., a cell with one or more CRISPR knockouts, and a base-edited cell. In some embodiments, the previously edited cell is a T-cell. In some embodiments, the previous modifications comprise gene knockouts in a T-cell, e.g., endogenous TCR (e.g., TRAC, TRBC), HLA Class I (B2M), PD1, CD52, CTLA-4, TIM-3, LAG-3, DGK. In some embodiments, a LSR system of the present invention is used to insert a TCR or CAR into a T-cell that has been previously modified. In some embodiments, the immune cells (e.g., T cells and NK cells) are previously modified with increased cytotoxic activities. As non-limiting examples, the T cells are genetically modified by a gene editing system, e.g., CRISPR/Cas system and base editing system. One or more genes (e.g., a TCR receptor gene, e.g., TRAC and TRBC) are inhibited in the modified T cells.

Exemplary diseases, disorders and clinical indications that can be treated using the present recombinases, systems and compositions include a hematopoietic stem cell (HSC) disease, disorder, or condition; a kidney disease, disorder, or condition; a liver disease, disorder, or condition; a lung disease, disorder, or condition; a skeletal muscle disease, disorder, or condition; a skin disease, disorder, or condition; a neurological disease, disorder, or condition; a heart disease, disorder, or condition; a spinal disease, an inflammatory disease, an infectious disease, a genetic defect, and a cancer. A cancer can be cancer of the cerebrum, cerebellum, adrenal gland, ovary, pancreas, parathyroid gland, hypophysis, testis, thyroid gland, breast, spleen, tonsil, thymus, lymph node, bone marrow, lung, cardiac muscle, esophagus, stomach, small intestine, colon, liver, salivary gland, kidney, prostate, blood, or other cell or tissue type, and can include multiple cancers.

### Administration

The composition and systems described herein may be used in vitro or in vivo. In some embodiments the system or components of the system are delivered to cells (e.g., mammalian cells, e.g., human cells), e.g., in vitro or in vivo. The skilled artisan will understand that the components of the LSR system may be delivered in the form of polypeptide, nucleic acid (e.g., DNA, RNA), and combinations thereof.

In some embodiments, the LSR system and/or components of the system are delivered as nucleic acids, e.g., DNA or mRNA. In some embodiments the system or components of the system are delivered as a combination of DNA and protein. In some embodiments the system or components of the system are delivered as a combination of RNA and protein. In some embodiments the recombinase polypeptide is delivered as a protein.

In some embodiments the system or components of the system are delivered to cells, e.g., mammalian cells or human cells, using a vector. The vector may be, e.g., a plasmid or a virus such as adenovirus, an AAV, a lentivirus or a retrovirus. In some embodiments delivery is in vivo, in vitro, ex vivo, or in situ.

In one embodiment, the compositions and systems described herein can be formulated in liposomes or other similar vesicles. Liposomes are spherical vesicle structures composed of a uni-or multilamellar lipid bilayer surrounding internal aqueous compartments and a relatively impermeable outer lipophilic phospholipid bilayer. Liposomes may be anionic, neutral or cationic. Liposomes are biocompatible, nontoxic, can deliver both hydrophilic and lipophilic drug molecules, protect their cargo from degradation by plasma enzymes, and transport their load across biological membranes and the blood brain barrier (BBB).

In some embodiments, a LSR system described herein is delivered to a tissue or cell from the cerebrum, cerebellum, adrenal gland, ovary, pancreas, parathyroid gland, hypophysis, testis, thyroid gland, breast, spleen, tonsil, thymus, lymph node, bone marrow, lung, cardiac muscle, esophagus, stomach, small intestine, colon, liver, salivary gland, kidney, prostate, blood, or other cell or tissue type.

In some embodiments, a LSR system described herein described herein is administered by enteral administration (e.g., oral, rectal, gastrointestinal, sublingual, sublabial, or buccal administration). In some embodiments, a Gene Writer™ system described herein is administered by parenteral administration (e.g., intravenous, intramuscular, subcutaneous, intradermal, epidural, intracerebral, intracerebroventricular, epicutaneous, nasal, intra-arterial, intra-articular, intracavernous, intraocular, intraosseous infusion, intraperitoneal, intrathecal, intrauterine, intravaginal, intravesical, perivascular, or transmucosal administration). In some embodiments, a LSR system described herein is administered by topical administration (e.g., transdermal administration).

### Kits

In one aspect, the invention provides kits containing any one or more of the elements disclosed in the above methods and compositions. In some embodiments, the kit comprises a vector system and instructions for using the kit. In some embodiments, the vector system comprises one or more insertion sites for inserting a guide sequence, wherein when expressed, the attP (or attB) sequence directs sequence-specific recombination by a large serine recombinase of heterologous DNA within a target sequence in a eukaryotic cell. Elements may be provided individually or in combinations, and may be provided in any suitable container, such as a vial, a bottle, or a tube. In some embodiments, the kit includes instructions in one or more languages, for example in more than one language.

In some embodiments, a kit comprises one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g., in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10. In some embodiments, the kit comprises one or more oligonucleotides corresponding to a guide sequence for insertion into a vector so as to operably link the guide sequence and a regulatory element. In some embodiments, the kit comprises a homologous recombination template polynucleotide.

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

### EXAMPLES

The following examples describe some of the preferred modes of making and practicing the present invention. However, it should be understood that these examples are for illustrative purposes only and are not meant to limit the scope of the invention.

### Example 1. Screening Novel Recombinant Large Serine Recombinases

A large number of large serine recombinases are sequenced from bacteriophages and the enzyme polypeptides are gathered for preparing a library of large serine recombinases. As described herein, novel large serine recombinases are derived from human gut metagenomes (Camarillo-Guerrero et al., Massive expansion of human gut bacteriophage diversity; Cell, 2021, 184:1098-1109;http://ftp.ebi.ac.uk/pub/databases/metagenomics/genome_sets/gut_phage_database/; the contents of which are incorporated herein by reference).

A library of vectors were prepared, each of which was designed to include an open reading frame of a candidate large serine recombinase from genomes in the Gut Phage Genome database (sequence identifiers are provided in Table 3), a nucleic acid sequence comprising about 300 bp downstream of the LSR encoding sequence in the phage genome and about 300 bp upstream of the LSR encoding sequence in the phase genome and a unique barcode that correlates to the LSR in the vector. The expression was controlled using a CMV promoter and a GFP reporter gene was incorporated to the vector. The vectors for different LSRs (e.g., LSRs defined by any one of SEQ ID NOs: 1-774 or codon-optimized LSR defined by any one of SEQ ID NOs: 775-1548) were pooled together for screening and identifying an active recombinase in the pooled library.

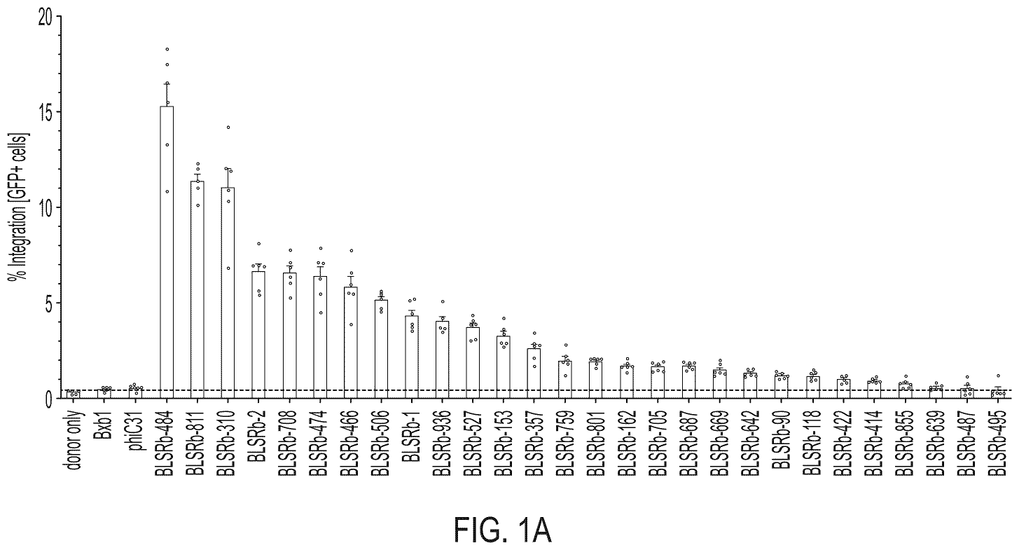

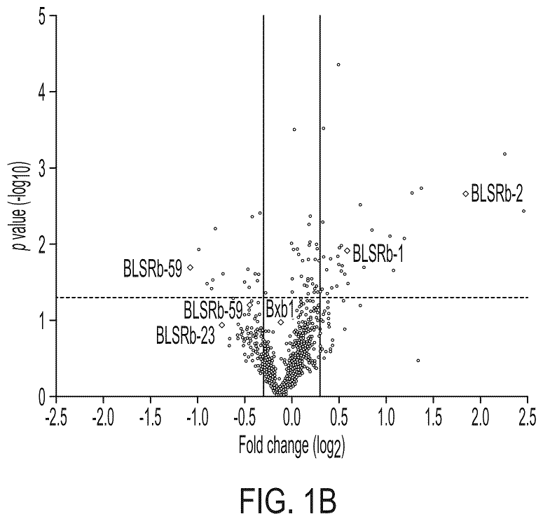

The vectors were transfected with HEK293 cells. Cells were cultured and harvested 1 week, 2 weeks or 3 weeks after the transfection. GFP expression indicated integration or recombinase activity. FIG. 1 illustrates exemplary large serine recombinases with high recombination or integration activity as measured by a GFP reporter assay.

Samples were prepared and sequenced using next-generation sequencing (NGS). Large serine recombinases showing high activity were identified by sequencing barcodes of the vectors. Using this approach, novel large serine recombinase enzymes were identified from different phage genomes

### Example 2. Evaluating Integration or Recombination Activity of Large Serine Recombinases in Human Cells

In this example, novel engineered large serine recombinase enzymes were recombinantly produced and tested for activity. The recombination or integration activity of novel large serine recombinases was tested in human cells. The large serine recombinases were used to target loci in HEK293T cells by transfection and tested for integration or recombination.

Briefly, HEK293T cells were plated in a 96-well plate. Cells were transfected with expression vectors comprising large serine recombinase under the control of a promoter and a cognate attP (or attB) site, 24 hours after plating. The vector further comprised a GFP reporter gene and a barcode for next generation sequencing.

GFP expression was evaluated and the presence of positive GFP expression validated serine recombinase activity in the target cell. Integration efficiency was identified by % GFP expression. As shown in FIG. 1A, several exemplary large serine recombinases showed integration as seen by GFP expression.

GFP expressing cells were harvested 72 hours post-transfection and total DNA was extracted. Sequencing was carried out and reads from each sample were identified on the basis of their associated unique barcode and aligned to a reference sequence. The barcodes were engineered to be situated between the attP and large serine recombinase sequences and sequencing is used to identify the cognate attB sites in the target genome. For example, as shown in FIG. 1B, exemplary pseudo attB sites were identified in human cells. PCR was used to amplify targeted insertions in the human genome.

The results showed that active large serine recombinases could integrate into the genome in human cells and lead to expression of heterologous DNA.

Similarly, in some embodiments, the barcodes are engineered to be situated between attB and large serine recombinase sequences and sequencing is used to identify the cognate attP sites in the target genome.

### Example 3: Mapping the Integration Sites of a Large Serine Recombinase

Active large serine recombinases identified from a database, e.g., using methods of examples 1 and 2, are further tested for the integration sites in a target genome.

A vector that expresses a large serine recombinase is transfected into target cells, with or without a heterologous sequence. After transfection, cells are harvested and genomic DNA samples are collected. The targeted insertions (TI) integrated randomly in human genome are amplified using PCR. The inserts are amplified and tested for sites of integration by flanking sequences, and recombinase activity is assayed.

Overall, the results from this example will show the sites of integration.

### Example 4: Testing Integration Efficiency Upon Cotransfection of Donor Containing attP Sites and LSR mRNA

In this example, exemplary LSR mRNA about 1.5 kb in length (SEQ ID NO: 377) and an exemplary DNA donor with attP sites that was about 6 kb in length were cotransfected into HEK293T cells. Briefly, 25,000 HEK293T cells per well of a 96 well plate were seeded and 24 h later, cells were transfected using varying amounts of plasmid donor (e.g., 50 ng or 200 ng) and varying amounts of LSR mRNA (e.g., 0, 10, 25, 50, 100 or 200 ng).

Transfection was carried out using exemplary transfection reagents and standard protocols, for example, 400 uL OPTIMEM, 100 uL of MessengerMax are mixed in a tube. In a second tube, X uL mRNA, y uL dsDNA donor without LSR is mixed with 5 uL-(x+y) uL of OPTIMEM. The contents of both tubes are mixed and incubated at room temperature for 5 minutes to add to cells.

Media is changed the day after transfection, and cells are split every 2-3 days. After 2 weeks of culturing, cells are harvested by trypsinizination and resuspended in PBS after washing. Flow cytometry was carried out (e.g., on an Attune instrument). Data was analyzed using FlowJo, gating on the forward and side scatter and gating on the GFP channel. WT untransfected cells were used as a negative control.

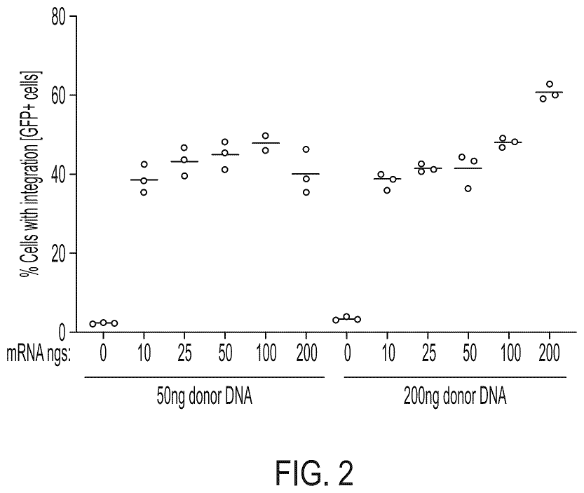

The results in FIG. 2 showed a dose dependent increase in integration of exemplary LSR-484. The highest activity observed was ˜60% insertion activity.

Overall, the results showed dose dependent increase in integration of LSR and up to about 60% integration efficiency was achieved.

### Example 5: Integration Efficiency Upon Nucleofection of LSR mRNA at High Doses in HEK293T Cells

In this example, 2×10⁵ HEK293T cells were nucleofected with an exemplary LSR mRNA of about 1.5 kb length (SEQ ID NO: 377) and a DNA donor with attP sites about 6 kb long. HEK293T cells were trypsinized and resuspending to single cell suspension. In some embodiments, other cell types such as K562 which grow in suspension are used without trypsizination.

Briefly, cells are counted and nucleofected using the RNA-DNA mix as described in Example 4 using standard protocols in a nucleofector, for example, Lonza. Varying amounts of mRNA (0, 100, 250, 500, 1000 or 2000 ng) and DNA donor (e.g., 1 μg, 2 μg or 3 μg). After nucleofection, cells are plated in 6 well plates and split every 2-3 days. After 2 weeks of culturing, cells are harvested, trypsinized, mixed, washed, spun, and resuspended in PBS. Flow cytometry was performed, for example, using an Attune instrument.

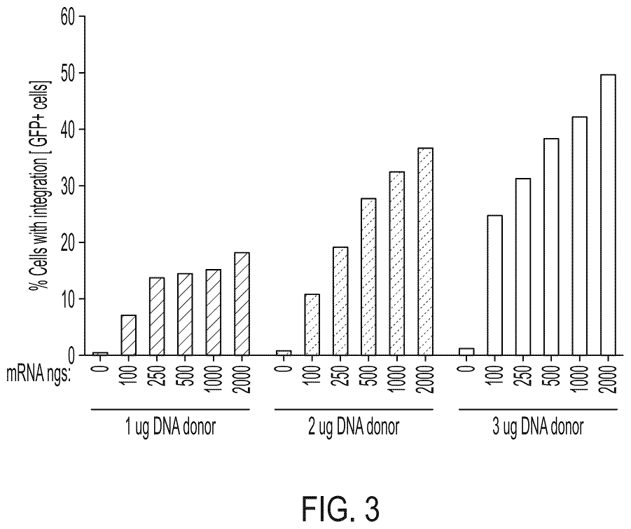

Flow cytometry data was analyzed using FlowJo, by gating on the forward and side scatter and then gating on the GFP channel. WT untransfected cells were used as a negative control. The results in FIG. 3 showed a dose dependent increase in integration, dependent on both the amount of mRNA and donor DNA.

About 50% integration was observed with 3 μg DNA.

Overall, nucleofection resulted in high integration in a dose-dependent manner.

### Example 6: Testing Integration Activity in Human Cells Using Exemplary LSRs

This Example evaluated integration activity in human K562 cells. Nucleofection assay was carried out in K562 cells using exemplary BLSRb-484 (SEQ ID NO: 377; pTI94 pMaxGFP core attP 70 bp, no LSR; mRNA 3435) and BLSRb-310 (SEQ ID NO: 239; pTI96 pMaxGFP core attP 70 bp, no LSR; mRNA 3432) recombinase.

2×10⁵ suspension cells were nucleofected using standard protocols in a nucleofector (e.g. Lonza). Cells were plated in 6 well plates and split every 2-3 days. After culture for about 2 weeks, cells were harvested, washed and resuspended in PBS. Flow cytometry was performed, for example, using an Attune instrument.

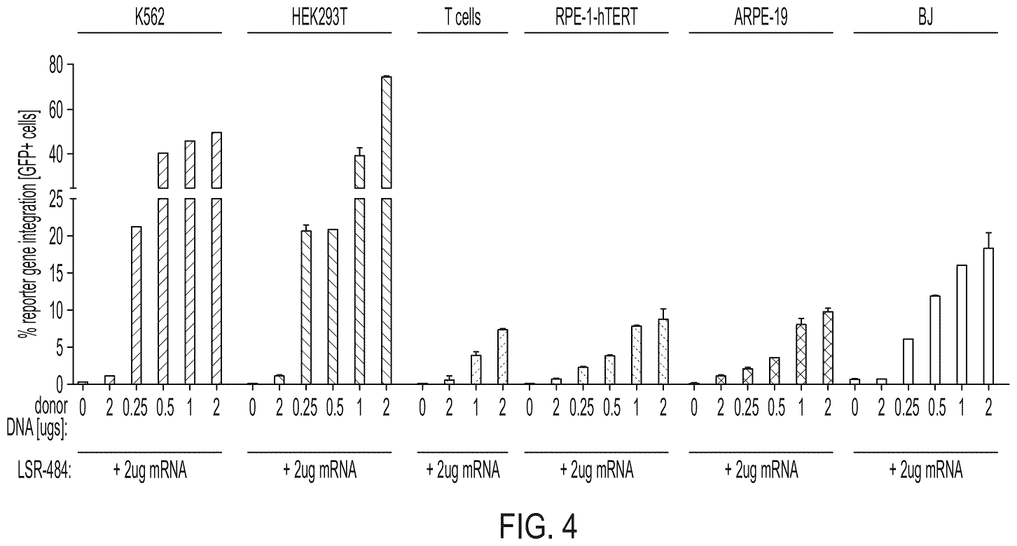

Flow cytometry data was analyzed using FlowJo, by garting on the forward and side scatter and then gating on the GFP channel. WT untransfected cells were used as a negative control. The results in FIG. 4 showed a dose dependent increase in integration, dependent on both the amount of mRNA and donor DNA.

The results showed that there was a dose dependent increase in integration activity, dependent on both amount of mRNA and donor DNA.

About 70% integration was observed with 4 μg DNA donor for LSR-484 and up to 35% integration with 4 μg DNA donor for LSR-310.

### Equivalents and Scope

Those skilled in the art will recognize, or be able to ascertain using no more than routine experimentation, many equivalents to the specific embodiments of the invention described herein. The scope of the present invention is not intended to be limited to the above Description, but rather is as set forth in the following claims.

1. A system for modifying DNA, the system comprising:
(a) a large serine recombinase having at least 70% identity to any one of the amino acid sequences listed in SEQ ID NOs: 1-774;
(b) a DNA recognition sequence comprising an attP or an attB attachment site; and/or
(c) a heterologous DNA sequence.

2. The system of claim 1, wherein the large serine recombinase has at least 80%, 85%, 90%, 95% or greater identity to any one of the amino acid sequences listed in SEQ ID NOs: 1-774.

3. The system of claim 1, wherein the large serine recombinase has at least 99% identity to any one of the amino acid sequences listed in SEQ ID NOs: 1-774.

4. The system of claim 1, wherein the large serine recombinase has 100% identity to any one of the amino acid sequences listed in SEQ ID NOs: 1-774.

5. The system of any one of the preceding claims, wherein the large serine recombinase has at least 70% identity to any one of polynucleotide sequences listed in SEQ ID NOs: 775-1548.

6. The system of any one of the preceding claims, wherein the large serine recombinase has at least 80%, 85%, 90%, 95% or greater identity to any one of the polynucleotide sequences listed in SEQ ID NOs: 775-1548.

7. The system of any one of the preceding claims, wherein the large serine recombinase has at least 99% identity to any one of the polynucleotide sequences listed in SEQ ID NOs: 775-1548.

8. The system of any one of the preceding claims, wherein the large serine recombinase has 100% identity to any one of the polynucleotide sequences listed in SEQ ID NOs: 775-1548.

9. The system of any one of the preceding claims, wherein the large serine recombinase is derived from a phage or bacterial genome.

10. The system of claim 9, wherein the phage or bacterial species is any one of the sources listed in SEQ ID NOs: 1-774.

11. The system of any one of the preceding claims, wherein the system comprises an attP site that recognizes a cognate attB site in the genome and causes recombination integrating the heterologous DNA in the genome.

12. The system of any one of the preceding claims, wherein the system comprises an attB site that recognizes a cognate attP site in the genome and causes recombination integrating the heterologous DNA in the genome.

13. The system of any one of the preceding claims, wherein the attP or attB site comprises a parapalindromic sequence.

14. The system of any one of the preceding claims, wherein the attP or attB sites are naturally occurring, i.e., pseudo attP or pseudo attB sites.

15. The system of any one of the preceding claims, wherein the attP or attB sites are engineered or optimized for expression in a target cell.

16. The system of any one of the preceding claims, wherein the heterologous DNA sequence is recombined or inserted into the target genome at one or more attP or attB sites.

17. The system of claim 16, wherein the heterologous DNA sequence is recombined or inserted into the target genome at a single attP or attB site.

18. The system of any one of the preceding claims comprised in one or more integrative vectors.

19. The system of claim 18, wherein the system is comprised in a single integrative vector.

20. The system of claim 18 or 19, wherein the vector is an adeno-associated virus (AAV) or lentivirus vector.

21. A method for modulating a genome in a cell, the method comprising:
(a) contacting the cell with a polypeptide encoding a serine recombinase enzyme having at least 70% identity to any one of the amino acid sequences listed in SEQ ID NOs: 1-774;
(b) a DNA recognition sequence comprising a first and a second attachment site; and/or
(c) a heterologous DNA sequence;
wherein the serine recombinase enzyme mediates site-specific recombination between the first and the second attachment site causing integration of heterologous DNA, thereby modulating the genome.

22. The method of claim 21, wherein at least one site is a pseudo attachment site.

23. The method of claim 21, wherein one or more sites is an engineered site.

24. The method of claim 23, wherein the first and second attachment sites are attP or attB sites.

25. The method of claim 24, wherein the attB site is in a target genome and the attP site sequence is in an integrative vector.

26. The method of claim 24, wherein the attP site sequence is in a target genome and the attB site sequence is in an integrative vector.

27. The method of any one of the preceding claims, wherein the site-specific recombination occurs at one or more sites in the cell.

28. The method of any one of the preceding claims, wherein the site-specific recombination occurs at a single site in the cell.

29. The method of any one of claims 21-28, wherein the site-specific recombination results in expression of a heterologous gene.

30. The method of any one of the preceding claims, wherein the recombination is carried out in a mammalian cell.

31. The method of claim 30, wherein the recombination is carried out in a human cell.

32. The method of any one of the preceding claims, wherein the recombination is carried out in a cultured cell.

33. The method of any one of claims 21-32, wherein the recombination is carried out in a primary cell.

34. The method of any one of the preceding claims, wherein the recombination is carried out in a non-dividing cell.

35. The method of any one of the preceding claims, wherein the recombination is carried out in an immune cell.

36. The cell of claim 35, wherein the immune cell is a T-cell, B-cell or NK cell.

37. The method of any one of the preceding claims, wherein the recombination is carried out in vivo.

38. The method of claim 37, wherein the in vivo recombination treats a genetic disease by repairing a genetic mutation and/or restoring a functional gene.

39. The method of claim 38, wherein the in vivo recombination treats a cancer by delivering a lethal or conditional lethal gene.

40. The method of claim 39, wherein the in vivo recombination results in genome editing by introducing one or more enzymes selected from a group consisting of a Cas enzyme, a base editor, deaminase and a reverse transcriptase.

41. The method of any one of claims 21-40, wherein the serine recombinase directs stable integration of the heterologous DNA.

42. The method of any one of claims 21-40, wherein the serine recombinase directs reversible integration of the heterologous DNA.

43. The method of any one of the preceding claims, wherein the heterologous DNA further comprises a Recombinase Directionality Factor (RDF) leading to excision of integrated DNA from the genome.

44. The method of any one of the preceding claims, wherein the promoter is constitutive or inducible.

45. The method of any one of the preceding claims, wherein the heterologous DNA integrated is between about 2 kb to about 40 kb in length.

46. An engineered cell produced by the method of any one of the preceding claims 21-45.

47. A method of treating a genetic disease or cancer, wherein the engineered cell of claim 46 is administered to a patient in need thereof.

48. The attP attachment site of claim 1, wherein the site comprises between 30 to 75 contiguous nucleotides from any one of SEQ ID NOs: 1549-2322, corresponding to its cognate LSR sequence as described in Table 3.
